SEQUENCE LISTING (108 sequences)

SEQ ID NO: 1 - protein, 1418 aa, Artificial Sequence
  Description of Artificial Sequence Synthetic polypeptide
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNRKRPAATKKAGQAKKKKGSYPYDVPDYAYPYDVPDYAYPYDVPDYA

SEQ ID NO: 2 - protein, 174 aa, Moraxella bovoculi
MSKAMYEAKERYAKKKMQENTKIDTLTDEQHDALAQLCAFRHKFHSNKDSLFLSESAFSGEFSFEMQSDENSKLREVGLPTIEWSFYDNSHIPDDSFREWFNFANYSELSETIQEQGLELDLDDDETYELVYDELYTEAMGEYEELNQDIEKYLRRIDEEHGTQYCPTGFARLR

SEQ ID NO: 3 - protein, 234 aa, Moraxella bovoculi
MYEIKLNDTLIHQTDDRVNAFVAYRYLLRRGDLPKCENIARMYYDGKVIKTDVIDHDSVHSDEQAKVSNNDIIKMAISELGVNNFKSLIKKQGYPFSNGHINSWFTDDPVKSKTMHNDEMYLVVQALIRACIIKEIDLYTEQLYNIIKSLPYDKRPNVVYSDQPLDPNNLDLSEPELWAEQVGECMRYAHNDQPCFYIGSTKRELRVNYIVPVIGVRDEIERVMTLEEVRNLHK

SEQ ID NO: 4 - protein, 92 aa, Moraxella bovoculi
MKIELSGGYICYSIEEDEVTIDMVEVTTKRQGIGSQLIDMVKDVAREVGLPIGLYAYPQDDSISQEDLIEFYFSNDFEYDPDDVDGRLMRWS

SEQ ID NO: 5 - protein, 170 aa, Moraxella bovoculi
MYEAKERYAKKKMQENTKIDTLTDEQHDALAQLCAFRHKFHSNKDSLFLSESAFSGEFSFEMQSDENSKLREVGLPTIEWSFYDNSHIPDDSFREWFNFANYSELSETIQEQGLELDLDDDETYELVYDELYTEAMGEYEELNQDIEKYLRRIDEEHGTQYCPTGFARLR

SEQ ID NO: 6 - protein, 322 aa, Moraxella bovoculi
MHHTIARMNAFNKAFANAKDCYKKMQAWHLLNKPKHAFFPMQNTPALDNGLAALYELRGGKEDAHILSILSRLYLYGAWRNTLGIYQLDEEIIKDCKELPDDTPTSIFLNLPDWCVYVDISSAQIATFDDGVAKHIKGFWAIYDIVEMNGINHDVLDFVVDTDTDDNVYVPQPFILSSGQSVAEVLDYGASLFDDDTSNTLIKGLLPYLLWLCVAEPDITYKGLPVSREELTRPKHSINKKTGAFVTPSEPFIYQIGERLGSEVRRYQSIIDGEQKRNRPHTKRPHIRRGHWHGYWQGTGQAKEFRVRWQPAVFVNSGRVSS

SEQ ID NO: 7 - protein, 319 aa, Moraxella bovoculi
MHHTIARMNAFNKAFGNAKDCYKKMQAWHLNNKPKHIFSPLQNTLSLNEGLAALYELHGGKEDEHILSILCCLYLYGTWRNTLGIYQLDEEIIKDCKELPDDTPTSIFLNLPDWCVYVDISSAKIATIDGGVAKHIKGFWAIYDNIEMHGVNHDVLNFIIDTDTDNNIYVPQSLILSSEMSVAESLDYGLTLFGYDESNELVKGMLPYLLWLCVAEPDITHKGLPVSREELTKPKHGINKKTGAFVTPSEPFIYQIGERLGGEVRRYQSLIDDEKNQNRHHTKRPHIRRGHWHGYWQGTGQAKEFKVRWQPAVFVNSGV

SEQ ID NO: 8 - protein, 168 aa, Moraxella bovoculi
MVGKSKIDWQSIDWTKTNAQIAQECGRAYNTVCKMRGKLGKSHQGAKSPRKDKGISRPQPHLNRLEYQALATAKAKASPKAGRFETNTKAKTWTLKSPDNKTYTFTNLMHFVRTNPHLFDPDDVVWRTKSNGVEWCRASSGLALLAKRKKAPLSWKGWRLISLTKDNK

SEQ ID NO: 9 - protein, 167 aa, Moraxella caviae
MIAHQKNRRADWESVDWTKHNDEIAQLLSRHPDSVAKMRTKFGAQGMAKRKPRRKYKVTRKAVPPPHTQELATAAAKISPKSGRYETNVNAKRWLIISPSGQRFEFSNLQHFVRNHPELFAKADTVWKRQGGKRGTGGEYCNASNGLAQAARLNIGWKGWQAKIIKG

SEQ ID NO: 10 - protein, 119 aa, Pseudomonas citronellolis
MSTQYTYQQIAEDFRLWSEYVDTAGEMSKDEFNSLSTEDKVRLQVEAFGEEKSPKFSTKVTTKPDFDGFQFYIEAGRDFDGDAYTEAYGVAVPTNIAARIQAQAAELNAGEWLLVEHEA

SEQ ID NO: 11 - protein, 425 aa, Eubacterium eligens
MRMERNKEIATSANLADSLTEQQCDVLEWASDMRHVMHKNSEALYDVNAPKHKEIKAFISDTHYSQNPNNLNKRLKDAGLPLIKWSFDDTRIPTNELAMILDNRRLEKSRKRQCIRTLEKANADIENYFAQIDAKYLTFYCPSGNKRVFSNQSKGVSVESRNVTIAEGYTIMGIESLKNNIAYLLHVYAGQISIEDIVAYVNEDIENRIYHMDASMAPQSVHKATNALVETGYIKPDTKELIYINLTKRGDAFVGSYCGTLNKIAASLSILPFNKAHKNDIVYYSYLIGWQRAKRELKPLIPKTVDFISNKIKEKQEMMYTGDDSNYAVEMEQTIIQSMNINSLPVVYEVKKGTYVIAEITTLFGKINVSIINSLFVGSAYTLTIPQYQYTAIIHMADNIDYGKIPYEVQKQLKAVVPVLMKLLQ

SEQ ID NO: 12 - protein, 322 aa, Moraxella catarrhalis
MHRTIARMHKFNKEFTNAKECYKKMQQAYLASKNKFAFFPMQHASLLDMSTAIAYEQTRSDPFSEKGVNALKTLNQLYLFGTWRYTLGIYCLDDEIIKDSKAIPDDTPTSIFLNLPEWCVYLDIASAKIAITQDNKTRHIKGFWAVYDLIEYNSKPQKAINFIIDTDSDDDIYLPLTLILDDDMTVEQSLSYADNKIGDGGSNELIKVLLPYLLWLCVAEPEIMHKGEPVSRANLDKPKYQTNKKTGVFIPPSEPFIYEVGSRLGGEIRHYQEQIEQGKHRQTSKKRPHIRRGHWHGHWHGTGQAKEFKIKWQPAIFVNSGV

SEQ ID NO: 13 - protein, 324 aa, Moraxella phage Mcat2
MKMHHTIARMQKFNKEFTNAKACYKKMQQAYLTSKNKFAFFPMQHASLLDMSTAIAYEQTRSDPFSEKGVNALKTLNQLYLFGTWRYTLGIYCLDDEIIKDSKAIPDDTPTSIFLNLPEWCVYLDIASAKIAITQDNKTRHIKGFWAVYDLIEYNSKPQKAINFIIDTDSDDDIYLPLTLILDDDMTVMQSLSYADNKIGDGGSNELIKVLLPYLLWLCVAEPEIMHKGEPVSRANLDKPKYQTNKKTGVFIPPSEPFIYEVGSRLGGEIRHYQEQIEQGKHRQTSKKRPHIRRGHWHGHWHGTGQAKEFKIKWQPAIFVNSGV

SEQ ID NO: 14 - protein, 357 aa, Moraxella osloensis
MIKDKDGNCIHGYDCYLAFNRKYPEAKELYKKLAEDQKNNPSKNGVYTTQQRIFQISDFLAEKTPSIQRLIADPRLYNPEKEPYTSFLSYVNGMPMFSAWRNSLDIYKIDPEIFEEMIKSPIPKDTPCEVFKRLPNFCVYVEMPRPTKFNELLMGNLNHLDKSFIVNGFWAYLGIEPNLHGNKNIQLNICLDYSSDIVQGNFDFLSMVIKEGLTVEEATELVFKQYDGNIETAKQDQRALFALLPILLWLCAEQPDITNIKDEPVTHEQLQQPKGSIHKKTGLFVPPNSPTYYNLGKRLGGEIRQYQELIKQDEKDRPTASKRPHIRKGHWHGYWKGTTGNKVFTPKWLSAIFVGFN

SEQ ID NO: 15 - protein, 174 aa, Moraxella catarrhalis
MIEYNSKPQKAINFIIDTDSDDDIYLPLTLILDDDMTVEQSLSYADNNIGDGGSNELIKILLPYLLWLCVAEPEIMHKGEPVSRANLDKPKYQTNKKTGVFIPPSEPFIYEVGSRLGGEIRHYQEQIEQGKHRQTSKKRPHIRRGHWHGHWHGTGQAKEFKIKWQPAIFVNAGV

SEQ ID NO: 16 - protein, 112 aa, Moraxella catarrhalis
MHRTIARMHKFNKEFTNAKECYKKMQQAYLASKNKFAFFPMQHASLLDMSTAIAYEQTRSDPFSEKGVNALKTLNQLYLFGTWRYTLGIYCLDSEIIKDSKAIPNDTPTSIF

SEQ ID NO: 17 - protein, 357 aa, Moraxella osloensis; identical to SEQ ID NO: 14

SEQ ID NO: 18 - protein, 360 aa, Moraxella osloensis
MLPYMTPFERYQAFVKTYPEAKETFKTMQAWYVANKPKNGIFVPSGNLYTMSPMLMKLVASKSKLAQSFTTMTDNDRLHLNYFWGLSLFGTWRYTLGVYQINDNLFDTLVKSPIPDDTPTSIFDKLPEWCVYIAFPEGKAINIKFNNGFADYEAFIFGFWVKLDTQNLTTSEGEQKIRVINFHLNLQTGIDNVFSNLQPLQLMIADDLSIKEAMQKHAKMVFEAYTPNHDFIVTQQNAKQDYDLTNKLLSLLLMLCAEAPDISKITGEPITKIELGKPKYTVNKRTGVFIPPQAPFLYEIGRRLGGDIKTTNDQLKNAGQGSGKGRRPHIRNAHYHGYWIGTGQNKQFKLNWIAPIFVNG

SEQ ID NO: 19 - protein, 361 aa, Moraxella osloensis
MTEEKYGGDPFEFMHAVNREFIDRKKDFNILAENYIDRHKTRGKQAYIDMGYLMGYIAHKYKINTHFQSEIPLGGVRDGSTVGKDAFSLAMFATWRLKPYVFEIDDDLFEQIKKSPIPFESPVSIFDNLPAWAVYVQLSNHELSIYTPAHEIIKLKCYGFWAYKAYSGEQLWLYMYPHVSQDDMTKTVNIQKFLPTSFLIINEKLDLFESLKKALEKMMDKKQEQHITPEIWDMHLNNSRLFLSALLLLCVERPQIEDSSLNEVDIASLSHLPPIHPKTKRFIAPNEPTKFFIGRRLGGQIRAFKAQESKGMPTGVTMQPHVRQAHWHGYRYGEGRKQFKLTFLPPIFVNMHAEDNLEERD

SEQ ID NO: 20 - protein, 165 aa, Rodentibacter ratti
MRRIDWHSVDWTKNNRQLADELGKAYDTVAKKRWELGQSGKAKDRAVRVDKGVSKTTCVPSPQQQRYATEMAKISPKSGKFETNIHSKKYKITSPDNQVFVITNLYQFVRDNKGLFLPTDVIFKRQGGTRGTGGEYCNATSGLLYISKHKTRTWKGWKCELLDSK

SEQ ID NO: 21 - protein, 174 aa, Ventosimonas gracilis
MVNQIKRRIKAASWEAMDWTKSNSQIAAETGKAYDTVAKRRVALGKSGMALQRSPRKDLKQLIARLQTPEMREKSKANQPLATQAAKASPKAGRGIDNVHAEDWHLLSPTGDSYKVRNLYEFVRANAHLFPPADVVWKRQGGARGTGGEYCNATAGILNIKGGKAKSWKGWRMV

SEQ ID NO: 22 - protein, 185 aa, Haemophilus haemolyticus
MDTVSRRRKQLARDTLLHQFRDWQNVDWSKTNKQLAIELGKSYDTVAKHRYQLGHGGEAKEREVRSDKGISKTTNIPSPELQKYATEQAQKSPNSGKFETNIHAKKWRITSPDNRVFVATNLYQFVRDNTALFLPSDVIFKRTGGKRGTGGEYCNATSGLLQAAASGRLWKGWKCKQIKKDNHEL

SEQ ID NO: 23 - protein, 252 aa, Aggregatibacter sp.
MSKIDWRAVDWSKRTIDLSRELNRTAKTVSDNRAKYAPETLKSHKNIDWLKIDWLKTTVQIAKELKVDFCTVAKARKKYAPETVIITPDWGKVDWTKNNRQLSQELGKSYNTVAKHRYQLGHSGEAKEREPKSNKGAPNPKMSHGRINQPKATAAAKNSPKSGKFETNIHAKKWRITSPDNQVFIVTNLYQFVRDHTHLFLPGDVIFKRTGGKRGTGGEYCNATNGLANAYTTKRGLWKGWRCKQIKEDKKR

SEQ ID NO: 24 - protein, 251 aa, Haemophilus haemolyticus
MSKIDWRTIDWSKRTIDLSRELNRTIKTVSDNRAKYAPETLKSHKNIDWLKIDWLKTTVQIAKELKVGFCAVAKARKKYAPETVITPNWDEVDWTKNNRQLAQELGKSYNTVAKKRCQLKQSGKAKERSVRIDKGQKKPQMAFGVVNQPLATKAAKTSPKSGKFETNIHAKKWRITSPDNRVFVATNLYQFVRDNTALFLPGDVIFKRTGGKRGTGGEYCNATSGLLQAAASGRLWKGWKCKQIKKDNHEL

SEQ ID NO: 25 - protein, 210 aa, Haemophilus haemolyticus
MSKIDWASVDWSMRSIDIARLLDVTIDTVSRRRKQLARDTLLHQFRDWQNVDWSKTNKQLAIELGKSYDTVAKHRYQLGHGGEAKEREVRSDKGISKTTNIPSPELQKYATEQAQKSPNSGKFETNIHAKKWRITSPDNRVFVATNLYQFVRDNTALFLPSDVIFKRTGGKRGTGGEYCNATSGLLQAAASGRLWKGWKCKQIKKDNHEL

SEQ ID NO: 26 - protein, 344 aa, Mannheimia varigena
MSRATKINWSELDWSKSTLELSKMLNVAGNFVSLKRRKYAPNTVRQKKAVDWSAIDWSKSTSDIAKQIGWSVANVSQKRKKYAPDTMGNLRNVGKYKRKVKPTVLKAPNGDILYMDSIKDFVIEYAHLFEAKHLISKNKKSGHIRQYCLAESALSSLRQKRVKKWQGWSLYEGFEEQSKLKRIDWDNVDWTKNNDQLAKELNRAYDTVAKKRYLLGKSGMATSRKEKADKGQKNPKKAIGAIKTQPIAKEWAKKSQKSGKFETNVHAKRWRLTREDGKCWEFTNLYHFVRTHTELFLPNDTVWKRTGGKRGTGGEYCNATSGLLNACRSRSKKWKGWKIEKIEN

SEQ ID NO: 27 - protein, 250 aa, Aggregatibacter sp.
MSKIDWRAVDWSKRTIDLSRELNRTAKTVSDNRAKYAPETLKSHKNIDWLKIDWLKTTVQIAKELKVDFCTAAKARKKYAPETVIITPDWDKVDWTKSNRQLSQELGKSYNTVAKHRYQLGHSGEAKEREPKSNKGVPNPKMSHGRINQPKATEAAKNSPKSGKFETNIHAKKWRITSPDNQVFVATNLYQFVRDHTHLFLPGDVIFKRTGGKRGTGGEYCNATNGLANASTTKREMWKGWKCEKIKEGK

SEQ ID NO: 28 - protein, 162 aa, Neisseria sp.
MPKYDWDKIDWRLSNHEIAAILQCSYDTVASKRYRLKVGKATKPKTRSDKGISRTTYLPPKEQQRRAVEAAKASPKAGRGETNCHAKRWRLTDPYGKQYEFSNLHHFIRCNNNLFTRKDVVWKRTGSNGGGEYCNASAGLQNVVAGKSPAWKGWEIEEITND

SEQ ID NO: 29 - protein, 383 aa, Serratia ficaria
MRLLICLTLSRSRKTGALPMAGRINSRAEAEAYVAGDLVECLECGKKFAFLPVHIKRMHGLNAEEYRERYNIPAGIPLAGKAYREMQRQKLVAMQKDGILDYSHLPKAEKAARRAGRGDKRDFDRQSQSHIMKLVNESGRAYRKTKSLFTPTAADNSIARVGPSYEQIEFIKNNAHKMSASEMQRELGISRKVIKRRADKLGLSLLKGKPPVSKPTLDWGSVDWSKSNKEIAASLGASYSAVKAMRRRLGVGPGKRAPMSNKGVKRNYSPEHLALIKKNAEKMRLAALSSSKISRTEHNIHAKKWTLVSPDGEVYRVVNLHNFIRENTELFNPEDVVWKLNGEEAEEGSRLWCRASQGIRSIKQRSVESWKGWKLLNPEDDEP

SEQ ID NO: 30 - protein, 164 aa, Acidovorax citrulli
MRKLADWAALDWAKPNAALAAEVGASVHTVAKRRTQHGVPMASPTWTRPDVAAINRRPERRAQSARTQPAATAAAKQSPAAGRGPDNVHALDWVLVSPSGERHQVRNLYDFVRSHSALFAEADVVWKRTGGKRGTGGEWCNATAGILNIKGGRAKSWKGWTLAQ

SEQ ID NO: 31 - protein, 164 aa, Acidovorax cattleyae
MRKLADWESLDWAKSNAVLAVEVGASIHTVAKRRTQHGVPTDSPTWKRPDVAAINQRPERRAQSARTQPAATAAARQSPAAGRGPENVHAVDWVLVSPSGERHQVRNLYDFVRSHAALFAEADVAWKRTGGKRGTGGEWCNATAGILNIKGGRAKSWKGWTLAQ

SEQ ID NO: 32 - protein, 170 aa, Moraxella bovoculi; identical to SEQ ID NO: 5

SEQ ID NO: 33 - protein, 66 aa, Moraxella bovoculi
MSETIQEQGLELDLDDDATYELVYDELYTEAMAEYEKLNQDIEKYLRRIDEEYGTQYCPTGFARLR

SEQ ID NO: 34 - protein, 132 aa, Eubacterium eligens
DSLTEQQCDVLEWASDMRHVMHKNSEALYDVNAPKHKEIKAFISDTHYSQNPNNLNKRLKDAGLPLIKWSFDDTRIPTNELAMILDNRRLEKSRKRQCIRTLEKANADIENYFAQIDAKYLTFYCPSGNKRV

SEQ ID NO: 35 - protein, 132 aa, Eubacterium eligens; identical to SEQ ID NO: 34

SEQ ID NO: 36 - protein, 132 aa, Eubacterium eligens; identical to SEQ ID NO: 34

SEQ ID NO: 37 - protein, 52 aa, Jaminaea rosea
KLDLREDEEGTVGLVDGRVRDEMRHEYEEMDQEVERQEVKIDEEEGTRILST

SEQ ID NO: 38 - protein, 234 aa, Moraxella bovoculi
MYEIKLNDTLIHQTDDRVNAFVAYRYLLRRGDLPKCENIARMYYDGKVIKTDVIDHDSVHSDEQAKVSNNDIIKMAISELGVNNFKSLIKKQGYPFSNGHINSWFTDDPVKSKTMHNDEMYLVVQSLIRACKIKEIDLYTEQLYNIIKSLPYDKRPNVVYSDQPLDPNNLDLSEPELWAEQVGECMRYAHNDQPCFYIGSTKRELRVNYIVPVIGVRDEIERVMTLEEVRNLHK

SEQ ID NO: 39 - protein, 95 aa, Moraxella caviae
MNKKSISQRVRRINNPKDKLALVQEWVSQRQSDFFSAFEQLEYAVGVDDLQQIHEAMDKIKDIAIKNYKAMPNIAEAMLVSKHYTVDLDEYEQEK

SEQ ID NO: 40 - protein, 65 aa, Pseudomonas otitidis [sequence omitted]

SEQ ID NO: 41 - protein, 79 aa, Pseudomonas aeruginosa
MNNDTEVLEQQIKAFELLADELKDRLPTLEILSPMYTAVMVTYDLIGKQLASRRAELIEILEEQYPGHAADLSIKNLCP

SEQ ID NO: 42 - protein, 106 aa, Pseudomonas aeruginosa
MIGSEKQVNWAKSIIEKEVEAWEAIGVDVREVAAFLRSISDARVIIDNRNLIHFQSSGISYSLESSPLNSPIFLRRFSACSVGFEEIPTALQRIRSVYTAKLLEDE

SEQ ID NO: 43 - protein, 132 aa, Pseudomonas aeruginosa
MSMELFHGSYEEISEIRDSGVFGGLFGAHEKETALSHGETLHRIISPLPLTDYALNYEIESAWEVALDVAGGDENVAEAIMAKACESDSNDGWELQRLRGVLAVRLGYTSVEMEDEHGTTWLCLPGCTVEKI

SEQ ID NO: 44 - protein, 156 aa, Moraxella catarrhalis BC8
MTTLYHGSHENTAPVIKIGFAAFLPADNVFDGIFANGDKNVARSHGDFIYAYEVDSIATNDDLDCDEAIQIIAKELYIDEETAAPIAEAVAYEESLAEFEEHIMPRSCGDCADFGWEMQRLRGVIARKLGFDAVECVDEHGVSHLIVNANIRGSIA

SEQ ID NO: 45 - protein, 124 aa, Pseudomonas aeruginosa
MAYEKTWHRDYAAESLKRAETSRWTQDANLEWTQLALECAQVVHLARQVGEELGNEKIIGIADTVLSTIEAHSQATYRRPCYKRITTAQTHLLAVTLLERFGSARRVANAVWQLTDDEIDQAKA

SEQ ID NO: 46 - protein, 115 aa, Moraxella catarrhalis BC8
MKLLNIKINEFAVTANTEAGDELYLQLPHTPDSQHSINHEPLDDDDFVKEVQEICDEYFGKGDRTLARLSYAGGQAYDSYTEEDGVYTTNTGDQFVEHSYADYYNVEVYCKADLV

SEQ ID NO: 47 - protein, 124 aa, Moraxella phage Mcat5
MKKIEMIEISQNRQNLTAFLHISEIKAINAKLADGVDVDKKSFDEICSIVLEQYQAKQISNKQASEIFETLAKANKSFKIEKFRCSHGYNEIYKYSPDHEAYLFYCKGGQGQLNKLIAENGRFM

SEQ ID NO: 48 - protein, 91 aa, Pseudomonas indica
MGVVVVLIIRLKARWSLHLERKLGEAGKAGIWEFHRSESSYTTDGRTTFRNAALRPAEPKEGQTVEVFICSDSREPEEQWRAVGEGVARYE

SEQ ID NO: 49 - protein, 151 aa, Pseudomonas indica
MLSVLFFWLYFYALFFIRFASSNKRARGRGMQRPALVSIALEWGMRRELMSRSFTTRIDHLQEVSRLGRGVARLRLGHSGRNLMPLILERRDGTGLTLKLDPKADPDEALRQLARGGIHVRVYSKYGERMRVVVDAPQAISILRDELVDRE

SEQ ID NO: 50 - protein, 67 aa, Pseudomonas aeruginosa
MTEEQFSALAELMRLRGGPGEDAARLVLVNGLKPTDAARKTGITPQAVNKTLSSCRRGIELAKRVFT

SEQ ID NO: 51 - protein, 190 aa, Moraxella bovoculi
MNNLKKTAITHDGVFAYKNTETVIGSVGRNDIVMAIDATHGEFNDKNFIIYADTNGNPIYLGYAYLDDNNDAHIDLAVGACNEDDDFDEKEIHEMIAEQMELAKRYQELGDTVHGTTRLAFDDDGYMTVRLDQQAYPDYRPENDDKHIMWRALALTATGKELEVFWLVEDYEDEEVNSWDFDIADDWREL

SEQ ID NO: 52 - protein, 66 aa, Moraxella catarrhalis
MSKNKTPDYVLRANANYRKKHTTNKSLQLHNEKDADIIQALQNETKSFNALMKDILRNHYNLNQNQ

SEQ ID NO: 53 - protein, 63 aa, Moraxella bovoculi
MNNPKTPEYTRKAIRAYEKNLVRKSVTFDVRKDDDMELLKMIEQDGRTFAQIARTALLEHLQK

SEQ ID NO: 54 - protein, 45 aa, Artificial Sequence
  Description of Artificial Sequence Synthetic polypeptide
GSGGGGSGPKKKRKVSSGYPYDVPDYAYPYDVPDYAYPYDVPDYA

SEQ ID NO: 55 - DNA, 4257 bp, Artificial Sequence
  Description of Artificial Sequence Synthetic polynucleotide
atgctgttccaggactttacccacctgtatccactgtccaagacagtgagatttgagctgaagcccatcgataggaccctggagcacatccacgccaagaacttcctgtctcaggacgagacaatggccgatatgcaccagaaggtgaaagtgatcctggacgattaccaccgcgacttcatcgccgatatgatgggcgaggtgaagctgaccaagctggccgagttctatgacgtgtacctgaagtttcggaagaacccaaaggacgatgagctgcagaagcagctgaaggatctgcaggccgtgctgagaaaggagatcgtgaagcccatcggcaatggcggcaagtataaggccggctacgacaggctgttcggcgccaagctgtttaaggacggcaaggagctgggcgatctggccaagttcgtgatcgcacaggagggagagagctccccaaagctggcccacctggcccacttcgagaagttttccacctatttcacaggctttcacgataaccggaagaatatgtattctgacgaggataagcacaccgccatcgcctaccgcctgatccacgagaacctgccccggtttatcgacaatctgcagatcctgaccacaatcaagcagaagcactctgccctgtacgatcagatcatcaacgagctgaccgccagcggcctggacgtgtctctggccagccacctggatggctatcacaagctgctgacacaggagggcatcaccgcctacaatacactgctgggaggaatctccggagaggcaggctctcctaagatccagggcatcaacgagctgatcaattctcaccacaaccagcactgccacaagagcgagagaatcgccaagctgaggccactgcacaagcagatcctgtccgacggcatgagcgtgtccttcctgccctctaagtttgccgacgatagcgagatgtgccaggccgtgaacgagttctatcgccactacgccgacgtgttcgccaaggtgcagagcctgttcgacggctttgacgatcaccagaaggatggcatctacgtggagcacaagaacctgaatgagctgtccaagcaggccttcggcgactttgcactgctgggacgcgtgctggacggatactatgtggatgtggtgaatccagagttcaacgagcggtttgccaaggccaagaccgacaatgccaaggccaagctgacaaaggagaaggataagttcatcaagggcgtgcactccctggcctctctggagcaggccatcgagcactataccgcaaggcacgacgatgagagcgtgcaggcaggcaagctgggacagtacttcaagcacggcctggccggagtggacaaccccatccagaagatccacaacaatcacagcaccatcaagggctttctggagagggagcgccctgcaggagagagagccctgccaaagatcaagtccggcaagaatcctgagatgacacagctgaggcagctgaaggagctgctggataacgccctgaatgtggcccacttcgccaagctgctgaccacaaagaccacactggacaatcaggatggcaacttctatggcgagtttggcgtgctgtacgacgagctggccaagatccccaccctgtataacaaggtgagagattacctgagccagaagcctttctccaccgagaagtacaagctgaactttggcaatccaacactgctgaatggctgggacctgaacaaggagaaggataatttcggcgtgatcctgcagaaggacggctgctactatctggccctgctggacaaggcccacaagaaggtgtttgataacgcccctaatacaggcaagagcatctatcagaagatgatctataagtacctggaggtgaggaagcagttccccaaggtgttcttttccaaggaggccatcgccatcaactaccacccttctaaggagctggtggagatcaaggacaagggccggcagagatccgacgatgagcgcctgaagctgtatcggtttatcctggagtgtctgaagatccaccctaagtacgataagaagttcgagggcgccatcggcgacatccagctgtttaagaaggataagaagggcagagaggtgccaatcagcgagaaggacctgttcgataagatcaacggcatcttttctagcaagcctaagctggagatggaggacttctttatcggcgagttcaagaggtataacccaagccaggacctggtggatcagtataatatctacaagaagatcgactccaacgataatcgcaagaaggagaatttctacaacaatcaccccaagtttaagaaggatctggtgcggtactattacgagtctatgtgcaagcacgaggagtgggaggagagcttcgagttttccaagaagctgcaggacatcggctgttacgtggatgtgaacgagctgtttaccgagatcgagacacggagactgaattataagatctccttctgcaacatcaatgccgactacatcgatgagctggtggagcagggccagctgtatctgttccagatctacaacaaggacttttccccaaaggcccacggcaagcccaatctgcacaccctgtacttcaaggccctgttttctgaggacaacctggccgatcctatctataagctgaatggcgaggcccagatcttctacagaaaggcctccctggacatgaacgagacaacaatccacagggccggcgaggtgctggagaacaagaatcccgataatcctaagaagagacagttcgtgtacgacatcatcaaggataagaggtacacacaggacaagttcatgctgcacgtgccaatcaccatgaactttggcgtgcagggcatgacaatcaaggagttcaataagaaggtgaaccagtctatccagcagtatgacgaggtgaacgtgatcggcatcgatcggggcgagagacacctgctgtacctgaccgtgatcaatagcaagggcgagatcctggagcagtgttccctgaacgacatcaccacagcctctgccaatggcacacagatgaccacaccttaccacaagatcctggataagagggagatcgagcgcctgaacgcccgggtgggatggggcgagatcgagacaatcaaggagctgaagtctggctatctgagccacgtggtgcaccagatcagccagctgatgctgaagtacaacgccatcgtggtgctggaggacctgaatttcggctttaagaggggccgctttaaggtggagaagcagatctatcagaacttcgagaatgccctgatcaagaagctgaaccacctggtgctgaaggacaaggccgacgatgagatcggctcttacaagaatgccctgcagctgaccaacaatttcacagatctgaagagcatcggcaagcagaccggcttcctgttttatgtgcccgcctggaacacctctaagatcgaccctgagacaggctttgtggatctgctgaagccaagatacgagaacatcgcccagagccaggccttctttggcaagttcgacaagatctgctataatgccgacaaggattacttcgagtttcacatcgactacgccaagtttaccgataaggccaagaatagccgccagatctggacaatctgttcccacggcgacaagcggtacgtgtacgataagacagccaaccagaataagggcgccgccaagggcatcaacgtgaatgatgagctgaagtccctgttcgcccgccaccacatcaacgagaagcagcccaacctggtcatggacatctgccagaacaatgataaggagtttcacaagtctctgatgtacctgctgaaaaccctgctggccctgcggtacagcaacgcctcctctgacgaggatttcatcctgtcccccgtggcaaacgacgagggcgtgttctttaatagcgccctggccgacgatacacagcctcagaatgccgatgccaacggcgcctaccacatcgccctgaagggcctgtggctgctgaatgagctgaagaactccgacgatctgaacaaggtgaagctggccatcgacaatcagacctggctgaatttcgcccagaacaggaaaaggccggcggccacgaaaaaggccggccaggcaaaaaagaaaaagggatcctacccatacgatgttccagattacgcttatccctacgacgtgcctgattatgcatacccatatgatgtccccgactatgcctaa

SEQ ID NO: 56 - protein, 6 aa, Artificial Sequence
  Description of Artificial Sequence Synthetic 6xHis tag
HHHHHH

SEQ ID NO: 57 - DNA, 27 bp, Moraxella bovoculi
gcttcaatcttggcaagtgtttcatca

SEQ ID NO: 58 - DNA, 28 bp, Moraxella bovoculi
agataggcatttgaaaaagaatttatct

SEQ ID NO: 59 - DNA, 27 bp, Moraxella bovoculi
ttcgtcctttatacgcaccccttgctt

SEQ ID NO: 60 - DNA, 29 bp, Moraxella bovoculi
atggttaatgatgataacccagatttaat

SEQ ID NO: 61 - DNA, 28 bp, Moraxella bovoculi
tttagaaatcacggatcattatatatgt

SEQ ID NO: 62 - DNA, 27 bp, Moraxella bovoculi
atatccatctactaaccatcgcaaaaa

SEQ ID NO: 63 - DNA, 27 bp, Moraxella bovoculi
attgatgtaaacatcgatggtgtggtt

SEQ ID NO: 64 - DNA, 27 bp, Moraxella bovoculi
attggtttgtgtaacggggaaattaag

SEQ ID NO: 65 - DNA, 27 bp, Moraxella bovoculi
tcaaaaatggtagcatttgttaagaat

SEQ ID NO: 66 - DNA, 27 bp, Moraxella bovoculi
tgcaggtggtgaatcagcgacacattc

SEQ ID NO: 67 - DNA, 28 bp, Moraxella bovoculi
ctaaatgccgtgtcgttttggttcttat

SEQ ID NO: 68 - DNA, 27 bp, Moraxella bovoculi
atgaaatagagcaacagcagaacggta

SEQ ID NO: 69 - DNA, 27 bp, Moraxella bovoculi
attgatgtaaacatcgatggtgtggtt

SEQ ID NO: 70 - DNA, 28 bp, Moraxella bovoculi
ctaaatgccgtgtcgttttggttcttat

SEQ ID NO: 71 - DNA, 34 bp, Moraxella bovoculi
accccgttatctgccacggtggcgttggctttgt

SEQ ID NO: 72 - DNA, 34 bp, Moraxella bovoculi
acttcgcaacattggctatccaagtaacgcaaac

SEQ ID NO: 73 - DNA, 34 bp, Moraxella bovoculi
agccaagctggttcggttgcccttgcctttggat

SEQ ID NO: 74 - DNA, 34 bp, Moraxella bovoculi
atcggttttgcattcggctaaggatttgggtgta

SEQ ID NO: 75 - DNA, 34 bp, Moraxella bovoculi
atttttaagcaccacgccataatcgccaaacacc

SEQ ID NO: 76 - DNA, 34 bp, Moraxella bovoculi
caaagactgctttttaagccaatcatagtagcta

SEQ ID NO: 77 - DNA, 34 bp, Moraxella bovoculi
ccaacacgcctaagacacgatgacttgtttttag

SEQ ID NO: 78 - DNA, 34 bp, Moraxella bovoculi
tatctcttcagcttgctcacgccaacccgcctgc

SEQ ID NO: 79 - DNA, 34 bp, Moraxella bovoculi
tggtgaattttcttttgagatgcagtctgatgaa

SEQ ID NO: 80 - DNA, 34 bp, Moraxella bovoculi
tttttcttgatcgatagacgactgattaaacaag

SEQ ID NO: 81 - DNA, 86 bp, Artificial Sequence
  Description of Artificial Sequence Synthetic oligonucleotide
tcacttcactgctggtggccactgctggtggccactgctggtggccactgctggtggccactgctggtggccactgctggtggcca

SEQ ID NO: 82 - DNA, 86 bp, Artificial Sequence
  Description of Artificial Sequence Synthetic oligonucleotide
tggccaccagcagtggccaccagcagtggccaccagcagtggccaccagcagtggccaccagcagtggccaccagcagtgaagtga

SEQ ID NO: 83 - DNA, 714 bp, Artificial Sequence
  Description of Artificial Sequence Synthetic polynucleotide
atgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaa

SEQ ID NO: 84 - DNA, 678 bp, Artificial Sequence
  Description of Artificial Sequence Synthetic polynucleotide
atggcgagtagcgaagacgttatcaaagagttcatgcgtttcaaagttcgtatggaaggttccgttaacggtcacgagttcgaaatcgaaggtgaaggtgaaggtcgtccgtacgaaggtactcagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtactaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaa

SEQ ID NO: 85 - DNA, 36 bp, Artificial Sequence
  Description of Artificial Sequence Synthetic oligonucleotide
gtctaacgaccttttaaatttctactgtttgtagat

SEQ ID NO: 86 - DNA, 24 bp, Artificial Sequence
  Description of Artificial Sequence Synthetic oligonucleotide
cactggagttgtcccaattcttgt

SEQ ID NO: 87 - DNA, 24 bp, Artificial Sequence
  Description of Artificial Sequence Synthetic oligonucleotide
aaagttcgtatggaaggttccgtt

SEQ ID NO: 88 - DNA, 48 bp, Artificial Sequence
  Description of Artificial Sequence Synthetic oligonucleotide
taatacgactcactataggctaacgaccttttaaatttctactgtttg

SEQ ID NO: 89 - DNA, 50 bp, Artificial Sequence
  Description of Artificial Sequence Synthetic oligonucleotide
tttccaatgatgagcactttatctacaaacagtagaaatttaaaaggtcg

SEQ ID NO: 90 - DNA, 49 bp, Artificial Sequence
  Description of Artificial Sequence Synthetic oligonucleotide
taatacgactcactataggtttcaaagattaaataatttctactaagtg

SEQ ID NO: 91 - DNA, 56 bp, Artificial Sequence
  Description of Artificial Sequence Synthetic oligonucleotide
tttccaatgatgagcactttatctacacttagtagaaattatttaatctttgaaac

SEQ ID NO: 92 - DNA, 45 bp, Artificial Sequence
  Description of Artificial Sequence Synthetic oligonucleotide
taatacgactcactataggtcaaaagacctttttaatttctactc

SEQ ID NO: 93 - DNA, 55 bp, Artificial Sequence
  Description of Artificial Sequence Synthetic oligonucleotide
tttccaatgatgagcactttatctacaagagtagaaattaaaaaggtcttttgac

SEQ ID NO: 94 - DNA, 100 bp, Artificial Sequence
  Description of Artificial Sequence Synthetic polynucleotide
ctcccttagccatccgagtggacgacgtcctccttcggatgcccaggtcggaccgcgaggaggtggagatgccatgccgaccctttccaatgatgagcac

SEQ ID NO: 95 - DNA, 56 bp, Artificial Sequence
  Description of Artificial Sequence Synthetic oligonucleotide
ggctaacgaccttttaaatttctactgtttgtagataaagtgctcatcattggaaa

SEQ ID NO: 96 - DNA, 57 bp, Artificial Sequence
  Description of Artificial Sequence Synthetic oligonucleotide
ggtttcaaagattaaataatttctactaagtgtagataaagtgctcatcattggaaa

SEQ ID NO: 97 - DNA, 56 bp, Artificial Sequence
  Description of Artificial Sequence Synthetic oligonucleotide
ggtcaaaagacctttttaatttctactcttgtagataaagtgctcatcattggaaa

SEQ ID NO: 98 - DNA, 2185 bp, Artificial Sequence
  Description of Artificial Sequence Synthetic polynucleotide
aattctaaagatctttgacagctagctcagtcctaggtataatactagtgcctctacctgcttcggccgataaagccgacgataatactcccaaagcccgccgaaaggcgggcttttttttggatccttactcgagtctagactgcaggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcagaatttcagataaaaaaaatccttagctttcgctaaggatgatttctgg

SEQ ID NO: 99 - DNA, 3046 bp, Artificial Sequence
  Description of Artificial Sequence Synthetic polynucleotide
gaattcaactgcaggcactgcccatggacctcggtaccgaatagctagccggtaatgcattcgctagagctcctaaagcatgcgacctgcaaccggtctgtcacgtacgtcgccaccgtcgacgtcgttcgtaagtagcctagataaataaaataatcagttaaccgcgagccccatgcgagagtagggaactgccaggcatttcagccaaaaaacttaagaccgccggtcttgtccactaccttgcagtaatgcggtggacaggatcggcggttttcttttctcttctcaaccgccgggagcggatttgaacgttgcgaagcaacggcccggagggtggcgggcaggacgcccgccataaactgccaggcatcaaattaagcagaaggccatcctgacggatggcctttttgcgtttctacaaactctgcggtaatacggttatccacagaatcaggggataacgcaggaaagaacatgtgagcaaaaggccagcaaaaggccaggaaccgtaaaaaggccgcgttgctggcgtttttccacaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattgtggatataccttactcgagttagccttgataggacgtcttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcgttgatagagttattttaccactccctatcagtgatagagaaaa

SEQ ID NO: 100 - DNA, 5275 bp, Artificial Sequence
  Description of Artificial Sequence Synthetic polynucleotide
gaattctaaagatctggcacgtaagaggttccaactttcaccataatgaaacatactagagaaagaggagaaatactagatggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcaggctgcaaacgacgaaaactacgctttagtagcttaataactctgatagtgctagtgtagatccctactagagccaggcatcaaataaaacgaaaggctcagtcgaaagactgggcctttcgttttatctgttgtttgtcggtgaacgctctctactagagtcacactggctcaccttcgggtgggcctttctgcgtttatatattgcttagaataatcgatctgcggccgcagagagtgtagcttacctagtcatcgaaagctttgctacagcggatagaattgtgagcggataacaattgacattgtgagcggataacaagatactactagtgtctaacgaccttttaaatttctactgtttgtagatcgatgtgacatcaagtgctacggggtctaacgaccttttaaatttctactgtttgtagatcaaagcccgccgaaaggcgggcttttttttgtggatataccttactcgagttagccttgatagattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatatatcggatccttagtaaacctgcaggcactgcccatggacctcggtaccgaatagctagccggtaatgcattcgctagagctcctaaagcatgcgacctgcaaccggtctgtcacgtacgtcgccaccgtcgacgtcgttcgtaagtagcctagataaataaaataatcagttaaccgcgagccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctgcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcagaatttcagataaaaaaaatccttagctttcgctaaggatgatttctg

SEQ ID NO: 101 - DNA, 5335 bp, Artificial Sequence
  Description of Artificial Sequence Synthetic polynucleotide
gaattctaaagatctggcacgtaagaggttccaactttcaccataatgaaacatactagagaaagaggagaaatactagatggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcaggctgcaaacgacgaaaactacgctttagtagcttaataactctgatagtgctagtgtagatccctactagagccaggcatcaaataaaacgaaaggctcagtcgaaagactgggcctttcgttttatctgttgtttgtcggtgaacgctctctactagagtcacactggctcaccttcgggtgggcctttctgcgtttatatattgcttagaataatcgatctgcggccgcagagagtgtagcttacctagtcatcgaaagctttgctacagcggatagaattgtgagcggataacaattgacattgtgagcggataacaagatactactagtgtctaacgaccttttaaatttctactgtttgtagataaagttcgtatggaaggttccgttgtctaacgaccttttaaatttctactgtttgtagatcactggagttgtcccaattcttgtgtctaacgaccttttaaatttctactgtttgtagatcaaagcccgccgaaaggcgggcttttttttgtggatataccttactcgagttagccttgatagattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatatatcggatccttagtaaacctgcaggcactgcccatggacctcggtaccgaatagctagccggtaatgcattcgctagagctcctaaagcatgcgacctgcaaccggtctgtcacgtacgtcgccaccgtcgacgtcgttcgtaagtagcctagataaataaaataatcagttaaccgcgagccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctgcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcagaatttcagataaaaaaaatccttagctttcgctaaggatgatttctg

SEQ ID NO: 102 - protein, 10 aa, Artificial Sequence
  Description of Artificial Sequence Synthetic 10xHis tag
HHHHHHHHHH

SEQ ID NO: 103 - protein, 188 aa, Eubacterium sp.
MKENRFCVVCGTKLHERQIKYCSKKCMGIGKQNYKICPVCGKQFKDAATNDTVCCSHECSKTHREQLHKSGIYDGSIEKMREGFSEKIVEIGPEKHWFSKHWVIESPSGQVYECDNLMHFIRTHPDLFDGTPTQAFDGFQKIKATREGKRPKAPSKSWKGWHLISYSENRNKYRKGETDGRKKSKDSI

SEQ ID NO: 104 - protein, 166 aa, Clostridium bolteae
MSKRPPKSTKICVVCGKTFPCFPSDKTVTCGKECSKIHRSRTHMGLSNAWSEESRTKKAAQGKTANLALGTPAAQKSPKSGKFLTNINAKDWHLISPDGKEYKFHCLNYWLRENCDKLFGCMPDSKEFKNVSTGLAGAKRAMLGKNYRCCTYKGWKVIPTEHDIKK

SEQ ID NO: 105 - protein, 330 aa, Leptospira phage
MKRKSSRRIKTRELLRPEQICTQVLGDDLDYYYSKFRIIFNAKGKTIPNWPDFCFAPIGCFFPFVFNENPDVDAPVLAEKVNLVSRLAGLIPWAIEKSIFRFSPTLLEYLIESGTPEKLPSQVLKKLPFWSIYIETPALADSVCIGFFAYLESNEGNDELRLILDTKAGPISLFLHLVEGNLDDSFEKATRLIKPRLELMNRANPDFDLEEMKDYSIHIYKTLLPLVLYICAENAEIRGNDSYAARLEKFQNIDLLNLKEAERPTIWNVGNSFDKEYKAFVERESSSSGLSNSKRPHLRRAHWHSFWRGKRNSSDRELILHFLSDISVNS

SEQ ID NO: 106 - protein, 363 aa, Escherichia coli
MEMSQLKQPIFLKKIKKVINTTPGLEEQIFACRNKKRSDNPLLFIDRKDEERILMSRLQSQQKNEELASKLESLFHGNELSSPHSILCFIYWRYTKKIYRLSEDIISDVANTYVDNIPAQILKELPSWSIYVSAENLHTILPTSYPIHGFFFYPFLDNNGNIIRLFIIDDLKQSQGTTGLKEKNVDVVNNIIRIKDSREGLLDSRKMECIDGEVVVTVNEKLKDFRDREFNLLNAQISMVLYICSQINDIKEKNQFKRSEKHKKHVHTHHELPAQNIREWDVGIRMGQAIRQYRQAEPTGKERTTIGSKRPHIRRGHWHTYWTGSKKPELAHERKPRLIWLPPVPVNLEDVNKLPVVITPIDK

SEQ ID NO: 107 - RNA, 31 nt, Artificial Sequence
  Description of Artificial Sequence Synthetic oligonucleotide
cuaaaugccgugucguuuugguucuuauguc

SEQ ID NO: 108 - DNA, 35 bp, Moraxella bovoculi
attataagaaccaaaacgacacggcatttagcaaa

(19) United States
(12) Patent Application Publication
(10) Pub. No.: US 2021/0363206 A1
(43) Pub. Date: Nov. 25, 2021

(54) PROTEINS THAT INHIBIT CAS12A (CPF1), A CRIPR-CAS NUCLEASE

(71) Applicant: THE REGENTS OF THE UNIVERSITY OF CALIFORNIA, OAKLAND, CA (US)

(72) Inventors: Nicole Blackburn-Marino, Oakland, CA (US); Joseph Bondy-Denomy, Oakland, CA (US); Kyle E. Watters, Oakland, CA (US); Jennifer A. Doudna, Oakland, CA (US)

(21) Appl. No.: 17/252,947

(22) PCT Filed: Jun. 17, 2019
(86) PCT No.: PCT/US19/37545

§ 371 Date: Dec. 16, 2020

Related U.S. Application Data
(60) Provisional application No. 62/686,593, filed on Jun. 18, 2018.

(51) Int. Cl. C07K 14/47; A61K 47/60; C12N 15/86; A61K 47/18

(52) U.S. Cl. CPC C07K 14/4703; A61K 47/60; A61K 38/00; A61K 47/183; C12N 15/86

(57) ABSTRACT

Cas12a-inhibiting polypeptides and methods of their use are provided.

### CROSS-REFERENCES TO RELATED APPLICATIONS

The present application claims priority to U.S. Provisional Application No. 62/686,593, filed Jun. 18, 2018, the disclosure of which is incorporated herein in its entirety.

### STATEMENT AS TO RIGHTS TO INVENTIONS MADE UNDER FEDERALLY SPONSORED RESEARCH AND DEVELOPMENT

This invention was made with government support under contract no. HR0011-17-2-0043 awarded by the Defense Advanced Research Projects Agency. The government has certain rights in the invention.

### BACKGROUND OF THE INVENTION

The ability to prevent attack from viruses is a hallmark of cellular life. Bacteria employ multiple mechanisms to resist infection by bacterial viruses (phages), including restriction enzymes and CRISPR-Cas systems (Labrie, S. J., Samson, J. E., and Moineau, S. (2010). Nat Rev Micro, 8, 317-327). CRISPR arrays possess the sequence-specific remnants of previous encounters with mobile genetic elements as small spacer sequences located between their clustered regularly interspaced short palindromic repeats (Mojica, F. J. M et al. (2005). J. Mol. Evol., 60, 174-182). These spacers are utilized to generate guide RNAs that facilitate the binding and cleavage of a programmed target (Brouns, S. J. J et al. (2008). Science, 321, 960-964; Garneau, J. E. et al. (2010). Nature, 468, 67-71). CRISPR-associated (cas) genes that are required for immune function are often found adjacent to the CRISPR array (Marraffini, L. A. (2015) Nature, 526, 55-61; Wright, A. V., Nunez, J. K., and Doudna, J. A. (2016). Cell, 164, 29-44). Cas proteins not only carry out the destruction of a foreign genome (Garneau, J. E. et al. (2010). Nature, 468, 67-71), but also facilitate the production of mature CRISPR RNAs (crRNAs) (Deltcheva; Haurwitz, R. E et al. (2010). Science, 329, 1355-1358) and the acquisition of foreign sequences into the CRISPR array (Nunez, J. K. et al. (2014). Nat. Struct. Mol. Biol, 21, 528-534; Yosef, I., Goren, M. G., and Qimron, U. (2012). Nucleic Acids Research, 40, 5569-5576).

CRISPR-Cas adaptive immune systems are common and diverse in the bacterial world. Six different types (I-VI) have been identified across bacterial genomes (Abudayyeh, O. O et al. (2016). Science aaf5573; Makarova, K. S. et al. (2015). Nat Rev Micro, 13, 722-736). Nat Rev Micro, 13, 722-736), with the ability to cleave target DNA or RNA sequences as specified by the RNA guide. The facile programmability of CRISPR-Cas systems has been widely exploited, opening the door to many novel genetic technologies (Barrangou, R., and Doudna, J. A. (2016), Nature Biotechnology, 34, 933-941). Most of these technologies use Cas9 from Streptococcus pyogenes (Spy), together with an engineered single guide RNA as the foundation for such applications, including gene editing in animal cells (Cong, L. et al. (2013). Science 339, 819-823; Jinek, M. et al. (2012). Science, 337, 816-821; Mali, P. et al. (2013). Science, 339, 823-826; Qi, L. S. et al. (2013). Cell, 152, 1173-1183). Additionally, Cas9 orthologs within the II-A subtype have been investigated for gene editing applications (Ran, F. A. et al. (2015). Nature 520, 186-191), and new Class 2 CRISPR single protein effectors such as Cpf1 (Type V (Zetsche, B. et al. (2015). Cell, 163, 759-771)) and C2c2 (Type VI (Abudayyeh, 0.0 et al. (2016). Science aaf5573; East-Seletsky, A. et al. (2016). Nature 538, 270-273) are being characterized. Class 1 CRISPR-Cas systems (Type I, III, and IV) are RNA-guided multi-protein complexes and thus have been overlooked for most genomic applications due to their complexity. These systems are, however, the most common in nature, being found in nearly half of all bacteria and ˜85% of archaea (Makarova, K. S. et al. (2015). Nat Rev Micro, 13, 722-736).

In response to the bacterial war on phage infection, phages, in turn, often encode inhibitors of bacterial immune systems that enhance their ability to lyse their host bacterium or integrate into its genome (Samson, J. E. et al. (2013). Nat Rev Micro, 11, 675-687). The first examples of phage-encoded “anti-CRISPR” proteins came for the (Class 1) type I-E and I-F systems in Pseudomonas aeruginosa (Bondy-Denomy et al. (2013). Nature, 493, 429-432; Pawluk, A. et al. (2014). mBio 5, e00896). Remarkably, ten type I-F anti-CRISPR and four type I-E anti-CRISPR genes have been discovered to date (Pawluk, A. et al. (2016). Nature Microbiology, 1, 1-6), all of which encode distinct, small proteins (50-150 amino acids), previously of unknown function. Biochemical investigation of four I-F anti-CRISPR proteins revealed that they directly interact with different Cas proteins in the multi-protein CRISPR-Cas complex to prevent either the recognition or cleavage of target DNA (Bondy-Denomy, J et al. (2015). Nature, 526, 136-139). Each protein has a distinct sequence, structure, and mode of action (Maxwell, K. L. et al. (2016). Nature Communications, 7, 13134; Wang, X. (2016). Nat. Struct. Mol. Biol 23, 868-870).

### BRIEF SUMMARY OF THE INVENTION

In some embodiments, methods of inhibiting a Cas12a polypeptide are provided. In some embodiments, the methods comprise: contacting a Cas12a-inhibiting polypeptide to the Cas12a polypeptide, wherein: the Cas12a-inhibiting polypeptide is substantially (e.g., at least 60%, 70%, 80%, 90%, 95%, 99%) identical to any one or more of SEQ ID NO: 2-53, thereby inhibiting the Cas12a polypeptide.

In some embodiments, the contacting occurs in vitro. In some embodiments, the contacting occurs in a cell. In some embodiments, the contacting comprises introducing the Cas12a-inhibiting polypeptide into the cell. In some embodiments, the Cas12a-inhibiting polypeptide is heterologous to the cell. In some embodiments, the Cas12a polypeptide is present in the cell prior to the contacting. In some embodiments, the Cas12a-inhibiting polypeptide comprises or consists of one of SEQ ID NO: 2-53. In some embodiments, the Cas12a-inhibiting polypeptide is substantially (e.g., at least 60%, 70%, 80%, 90%, 95%, 99%) identical to any one or more of SEQ ID NO: 2-53. In some embodiments, the cell comprises the Cas12a polypeptide before the introducing.

In some embodiments, the cell comprises a heterologous expression cassette comprising a promoter operably linked to a polynucleotide encoding the Cas12a polypeptide. In some embodiments, the promoter is inducible and the method comprises contacting the cell with an agent or condition that induces expression of the Cas12a polypeptide in the cell prior to the introducing.

In some embodiments, the Cas12a polypeptide is introduced to the cell when or after the Cas12a-inhibiting polypeptide is introduced to the cell. In some embodiments, the promoter is inducible and the method comprises contacting the cell with an agent or condition that induces expression of the Cas12a polypeptide in the cell after to the introducing.

In some embodiments, the introducing comprises expressing the Cas12a-inhibiting polypeptide in the cell from an expression cassette that is present in the cell and heterologous to the cell, wherein the expression cassette comprises a promoter operably linked to a polynucleotide encoding the Cas12a-inhibiting polypeptide. In some embodiments, the promoter is an inducible promoter and the introducing comprises contacting the cell with an agent that induces expression of the Cas12a-inhibiting polypeptide.

In some embodiments, the introducing comprises introducing an RNA encoding the Cas12a-inhibiting polypeptide into the cell and expressing the Cas12a-inhibiting polypeptide in the cell from the RNA.

In some embodiments, the introducing comprises inserting the Cas12a-inhibiting polypeptide into the cell or contacting the cell with the Cas12a-inhibiting polypeptide.

In some embodiments, the cell is a eukaryotic cell. In some embodiments, the cell is a mammalian cell or a plant cell. In some embodiments, the cell is a human cell. In some embodiments, the cell is a blood or an induced pluripotent stem cell.

In some embodiments, the method occurs ex vivo. In some embodiments, the cells are introduced into a mammal after the introducing and contacting. In some embodiments, the cells are autologous to the mammal.

In some embodiments, the cell is a prokaryotic cell.

Also provided is a cell comprising a Cas12a-inhibiting polypeptide, wherein the Cas12a-inhibiting polypeptide is heterologous to the cell and the Cas12a-inhibiting polypeptide is substantially (e.g., at least 60%, 70%, 80%, 90%, 95%, 99%) identical to any one or more of SEQ ID NO: 2-53. In some embodiments, the Cas12a-inhibiting polypeptide comprises or consists of one of SEQ ID NO: 2-53. In some embodiments, the cell is a eukaryotic cell. In some embodiments, the cell is a mammalian cell or a plant cell. In some embodiments, the cell is a human cell. In some embodiments, the cell is a prokaryotic cell. In some embodiments, the cell is a fungal cell.

Also provided is a polynucleotide comprising a nucleic acid encoding a Cas12a-inhibiting polypeptide, wherein the Cas12a-inhibiting polypeptide is substantially (e.g., at least 60%, 70%, 80%, 90%, 95%, 99%) identical to any one or more of SEQ ID NO: 2-53. In some embodiments, the Cas12a-inhibiting polypeptide comprises or consists of one of SEQ ID NO: 2-53. In some embodiments, the polynucleotide comprises an expression cassette, the expression cassette comprising a promoter operably linked to the nucleic acid. In some embodiments, the promoter is heterologous to the polynucleotide encoding the Cas12a-inhibiting polypeptide. In some embodiments, the promoter is inducible.

In some embodiments, the polynucleotide is DNA or RNA. The polynucleotide may be, for example, mRNA. In some aspects, the mRNA may be chemically modified (See e.g. Kormann, et al., (2011) Nature Biotechnology 29(2): 154-157).

Also provided is a vector comprising the expression cassette as described above or elsewhere herein. In some embodiments, the vector is a viral vector.

Also provided is a Cas12a-inhibiting polypeptide, wherein the Cas12a-inhibiting polypeptide comprises or consists of an amino acid sequence substantially (e.g., at least 60%, 70%, 80%, 90%, 95%, 99%) identical to any one or more of SEQ ID NO: 2-53. In some embodiments, the Cas12a-inhibiting polypeptide comprises or consists of one of SEQ ID NO: 2-53. In some embodiments, the amino acid sequence is linked to a heterologous protein sequence. In some embodiments, the heterologous protein sequence extends the circulating half-life of the polypeptide In some embodiments, the amino acid sequence is linked to an antibody Fc domain or human serum albumin. In some embodiments, the polypeptide is PEGylated and/or comprises at least one non-naturally-encoded amino acid.

Also provided is a pharmaceutical composition comprising the polynucleotide as described above or elsewhere herein. Also provided is a pharmaceutical composition comprising the polynucleotide as described above or elsewhere herein.

Also provided is a delivery vehicle comprising the polynucleotide as described above or elsewhere herein or the polynucleotide as described above or elsewhere herein. In some embodiments, the delivery vehicle is a liposome or nanoparticle.

### BRIEF DESCRIPTION OF THE DRAWINGS

FIG. 1: The discovery of a widespread Type I inhibitor. (A) The associations of novel Type I-E (IE5-7) and Type I-F (IF11-12) anti-CRISPRs with anti-CRISPR associated (aca1, aca4) genes in Pseudomonas sp. AcrIE4-7 is a chimera of two previously characterized Type I anti-CRISPRs (IE4 and IF7), and orf1_{Pse} and orf2_{Pae} did not manifest anti-CRISPR activity. (B) Phage plaque assays to assess CRISPR-Cas inhibition. Ten-fold serial dilutions of a Type I-E or Type I-F CRISPR-targeted phage (JBD8 or DMS3m, respectively) plated on lawns of Pseudomonas aeruginosa with naturally active Type I-E or Type I-F CRISPR-Cas systems. A restoration of phage plaquing (black) relative to the vector control indicates inhibition of CRISPR-Cas immunity by the expression of the specified plasmid-borne anti-CRISPR. Phages were titrated on ΔCRISPR-Cas strains to measure phage replication in the complete absence of CRISPR-Cas immunity (top row). (C) A midpoint rooted phylogenetic tree of full-length homologs of AcrIF11. Branch colors correspond to the class of bacteria in which each homolog was found (see legend). Select species have been labeled on the tree, see FIG. 3A for a comprehensive listing of species. Scale bar represents 0.1 substitutions per site.

FIG. 2. All Pseudomonas sp. ORFs from FIG. 1 are negative for anti-IC activity. (A) IC phage spotting data. Ten-fold serial dilutions of JBD30 phage were applied to bacterial lawns of P. aeruginosa LL77 and LL76 strains. LL77 is engineered to target JBD30 with a Type I-C CRISPR-Cas immune system, whereas LL76 lacks phage-targeting crRNA. (B) Phage plaque assays to test potential Type I-C inhibition by candidate genes.

FIG. 3. Full AcrIF11 tree with all species and aca1-aca7. (A) Midpoint rooted minimum-evolution phylogenetic tree of full-length AcrIF11 orthologs. Branches are labeled with species names. Species in which AcrIF11 is associated with a novel aca gene (aca4-7) are marked with asterisks. (B) A table of previously discovered aca genes (aca1-3) and novel aca genes found in this study (aca4-7). All aca proteins are predicted with high confidence to contain helix-turn-helix motifs as predicted by HHPred (Example 1 reference 24).

FIG. 4: Type V-A and Type I-C anti-CRISPR proteins identified in Moraxella. (A) Moraxella bovoculi exhibits intragenomic self-targeting, where a spacer encoded by a CRISPR-Cas12 system and its target protospacer exist within the same genome. (B) Schematic showing the presence of AcrIF11 orthologs in anti-CRISPR loci within Moraxella catarrhalis and the use of guilt-by-association to unveil novel Type V-A and Type I-C inhibitors in Moraxella bovoculi. Phage plaque assays with ten-fold serial diluations of the indicated phage to assess inhibition of CRISPR-Cas Type V-A (C), Type I-C (D), and Type I-F (E). Bacterial clearance (black) indicates phage replication. (C) P. aeruginosa PAO1 strain expressing MbCas12a, phage-targeting crRNA, and a candidate gene or vector control. “No crRNA” indicates full phage titer. (D) P. aeruginosa PAO1 strain engineered to express the Type I-C Cas proteins and crRNA system upon induction, and a candidate gene or vector control. Uninduced panel indicates full phage titer. (E) P. aeruginosa strain UCBPP-PA14 transformed with candidate gene or vector control. PA14ΔCRISPR-Cas strain indicates full phage titer.

FIG. 5: Percent identity between Pseudomonas and Moraxella Cas proteins. BLASTp was used to align the indicated protein orthologs between the Type I-C (A) and Type I-F (B) systems of Pseudomonas and Moraxella. The percent sequence identity between the proteins is shown, as well as an average value for the whole system.

FIG. 6: Functionality of novel Acr proteins against CRISPR-Cas systems they do not inhibit. Phage plaque assay to assess CRISPR-Cas inhibition. Ten-fold serial dilutions of (A) DMS3m or (B, C) JBD30 phage were applied to bacterial lawns of P. aeruginosa strain (A) UCBPP-PA14 expressing the Type I-F system, (B) PAO1 expressing the Type I-C system, or (C) PAO1 expressing the Type V-A system, transformed with candidate gene or vector control.

FIG. 7: AcrVA proteins have diverse phylogenetic distributions. Midpoint rooted phylogenetic reconstructions of AcrVA proteins. Full-length protein sequences of orthologs were generated using BLASTp searches for (A) AcrVA1 and (B) AcrVA2 and iterative psi-BLASTp for (C) AcrVA3. Scale bar indicates 0.1 substitutions per site.

FIG. 8: Protein sequence alignments of diverse orthologs of AcrVA2 and AcrVA3. The protein sequence of different orthologs of AcrVA2 (A) and AcrVA3 (B) were aligned and colored using Clustal Omega. The residue color indicates the following: red, hydrophobic; blue, acidic; magenta, basic; green, hydroxyl or sulfhydryl or amine group. Asterisk (*) indicates fully conserved residue. Colon (:) indicates conservation of strongly similar properties (>0.5 in the Gonnet PAM 250 matrix). Period (.) indicates conservation of weakly similar properties (<0.5 and >0 in Gonnet PAM 250 matrix). (A) AcrVA2 alignment includes orthologs from Moraxella bovoculi 58069, Moraxella catarrhalis BC8, Leptospira phage vB_LbrZ_5399-LE1, and E. coli (FinQ). (B) AcrVA3 alignment includes orthologs from Moraxella bovoculi 58069, Moraxella caviae, Neisseria sp. HMSC056A03, and Clostridium bolteae 90B7, and Eubacterium sp. An3.

FIG. 9: AcrVA1 blocks Cas12a-mediated gene editing in human cells. (A-C) Human cell U2-OS-EGFP disruption experiments to assess AcrVA-mediated inhibition of Cas12a activities. (A) Inhibition of MbCas12a activity with various AcrVA constructs; the “no filler” condition contained only plasmids for Cas12a and crRNA expression. (B) Comparisons between the inhibitory activities of AcrVA1 and AcrIIA4 against MbCas12a, Mb3Cas12a, and SpyCas9. Controls using “filler” plasmid in lieu of anti-CRISPR plasmids were included to equalize amounts of DNA. (C) Assessment of AcrVA1 activity against Cas12a orthologs, with AcrIIA4 used as control. For panels A-C, unless otherwise indicated, cells were co-transfected with a MbCas12a nuclease expression plasmid, an EGFP-targeting crRNA plasmid, and an anti-CRISPR expression plasmid. EGFP disruption activities were assessed by flow cytometry 52 hours post-transfection; background EGFP disruption is indicated by the red dashed line; error bars indicate s.e.m. for n=3. (D) Inhibition of Cas12a and SpyCas9 activities against endogenous sites in human cells was assessed by co-transfecting U2-OS cells with nuclease, anti-CRISPR, and crRNA or sgRNA expression plasmids (targeted to the RUNX1, DNMT1, or FANCF genes). Gene modification assessed by T7 endonuclease I (T7E1) assay 72 hours post-transfection; error bars indicate s.e.m. for n=3.

FIG. 10: Dose response curves of CRISPR nuclease inhibition by Acr proteins in human cells. Comparison between the inhibitory activities of AcrVA1 against MbCas12a and Mb3Cas12a, and AcrIIA4 against SpyCas9, across various levels of Acr expression. EGFP disruption activities assessed by flow cytometry 52 hours post-transfection; background EGFP disruption is indicated by the red dashed line; error bars indicate s.e.m. for n=3.

FIG. 11 shows a strategy to produce genomic fragments to test for anti-CRISPRs in self-targeting M. bovoculi genomes.

FIG. 12 shows how TXTL is used to test for anti-CRISPR activity of introduced genomic fragments from M. bovoculi. Inhibition of reporter cleavage is indicated by fluorescent reporter expression. A non-targeting control is also used as a control to observe the expected reporter expression levels without Cas12 activity.

FIG. 13 shows testing of genomic fragments from M. bovoculi. Fragments GF90, GF122, GF120, and GF112 (not shown) exhibited some level of anti-CRISPR activity.

FIG. 14 shows individual genes tested. Both plasmid (upper panel) and genomic amplicon (lower panel) sources of MbCas12 expression were used and inhibited by GF90 candidate 5 and GF122 candidates 9 and 10.

FIG. 15 shows biochemical validation of AcrVA1-3. (A) Moraxella bovoculi Cas12a (MbCas12a) in vitro dsDNA cleavage is inhibited by increasing concentrations of AcrVA1 and AcrVA2, but is not inhibited by AcrVA3. (B) LbCas12a, a Cas12a commonly used for gene editing and diagnostics, is inhibited by all three AcrVA proteins, although AcrVA3 only inhibits DNA cleavage at higher concentrations. (C) High concentrations of AcrVA1 also inhibits AsCas12a-mediated dsDNA cleavage, but AcrVA2 and AcrVA3 have no effect.

FIG. 16 shows human cell lines (HEK293T) stably expressing AcrVA1, AcrVA2, AcrVA3, BFP, or mCherry (right to left on each chart's x-axis). This plot represents data from RNP SpyCas9-sg1 (NLS) that was delivered targeting an inducible eGFP gene in the genome.

FIG. 17 shows human cell lines (HEK293T) stably expressing AcrVA1, AcrVA2, AcrVA3, BFP, or mCherry (right to left on each chart's x-axis). This plot represents data from RNP SpyCas9-sg2 (NLS) that was delivered targeting an inducible eGFP gene in the genome.

FIG. 18 shows human cell lines (HEK293T) stably expressing AcrVA1, AcrVA2, AcrVA3, BFP, or mCherry (right to left on each chart's x-axis). This plot represents data from RNP AsCas12a (NLS) that was delivered targeting an inducible eGFP gene in the genome.

FIG. 19 shows human cell lines (HEK293T) stably expressing AcrVA1, AcrVA2, AcrVA3, BFP, or mCherry (right to left on each chart's x-axis). This plot represents data from RNP LbCas12a (NLS) that was delivered targeting an inducible eGFP gene in the genome.

FIG. 20 shows human cell lines (HEK293T) stably expressing AcrVA1, AcrVA2, AcrVA3, BFP, or mCherry (right to left on each chart's x-axis). This plot represents data from RNP MbCas12a (NLS) that was delivered targeting an inducible eGFP gene in the genome.

FIG. 21. Ten-fold dilutions of phage JBD30, targeted by MbCas12a/Cpf1 in the presence or absence (AcrRNA) of a targeting crRNA. In the presence of AcrVA1 or AcrVA6, phage replication (black spots) is restored, via CRISPR inhibition. Truncation of AcrVA6 abolishes most anti-CRISPR function.

### DEFINITIONS

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. In some cases, conservatively modified variants of Cas9 or sgRNA can have an increased stability, assembly, or activity as described herein.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which are described in Altschul et al., (1990) J. Mol. Biol. 215: 403-410. Software for performing BLAST analyses is publicly available at the National Center for Biotechnology Information website. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR/Cas” system refers to a class of bacterial systems for defense against foreign nucleic acids. CRISPR/Cas systems are found in a wide range of eubacterial and archaeal organisms. CRISPR/Cas systems include type I, II, III, V, and VI sub-types. Wild-type V CRISPR/Cas systems utilize the RNA-mediated nuclease, Cas12a (formerly called Cpf1) in complex with guide and activating RNA to recognize and cleave foreign nucleic acid. See, e.g., Fonfara et al., Nature 532, 7600 (2016); Zetsche et al., Cell 163, 759-771 (2015). SEQ ID NO:1 is an exemplary Cas12a protein and SEQ ID NO:55 is an exemplary Cas12a coding sequence.

Several orthologs of Cas12a have been identified including those from Francisella novicida U112 (FnCpf1), Acidaminococcus sp. BV3L6 (AsCpf1), and Lachnospiraceae bacterium ND2006 (LbCpf1) (Endo, A., et al. Scientific Reports 6, 38169 (2016); Kim et al., Nature Biotechnology 34, 82016 (2016); Ma et al., Insect Biochemistry and Molecular Biology 83, 13-20 (2017); Zetsche et al., Cell 163, 759-771 2015; Zetsche et al., Nature Biotechnology 35, 31-34 (2016), as well as 16 others described in Zetsche, B., et al., BioRxiv Preprint (May 4, 2017); doi.org/10.1101/134015, which include Thiomicrospira sp. Xs5 (TsCpf1), Moraxella bovoculi AAX08_00205 (Mb2Cpf1), Moraxella bovoculi AAX11_00205 (Mb3Cpf1), and Butyrivibrio sp. NC3005 (BsCpf1).

In some embodiments, Cas12a protein can be nuclease defective. See, e.g., Swarts D. C., et al. Mol. Cell. 66:221-233 (2017). For example, the Cas12a protein can be a nicking endonuclease that nicks target DNA, but does not cause double strand breakage. Cas12a can also have nuclease domains deactivated to generate “dead Cas12a” (dCas12a), a programmable DNA-binding protein with no nuclease activity. For example, Cas12a from Francisella novicida (FnCas12a) can be rendered to a dCas12a by mutations E1006A and R1218A. In some embodiments, dCas12a DNA-binding is inhibited by the polypeptides described herein.

### DETAILED DESCRIPTION OF THE INVENTION

Several polypeptide inhibitors (“Cas12a-inhibiting polypeptides”) of Cas12a nuclease have been identified from phage and other mobile genetic elements in bacteria. The Cas12a-inhibiting polypeptides initially discovered from phage were designated AcrVA proteins (anti-CRISPR Type V-A).

The Cas12a-inhibiting polypeptides described herein can be used in many aspects to inhibit or control unwanted Cas12a activity. For example, one or more Cas12a-inhibiting polypeptide can be used to regulate Cas12a in genome editing, thereby allowing for some Cas12a activity prior to the introduction of the Cas12a-inhibiting polypeptide. This can be helpful, for example, in limiting off-target effects of Cas12a. This and other uses are described in more detail below.

As set forth in the examples and sequence listing, a large number of Cas12a-inhibiting polypeptides have been discovered. Examples of exemplary Cas12a-inhibiting polypeptides include proteins comprising any of SEQ ID NOs: 2-53, or substantially (e.g., at least 50, 60, 70, 75, 80, 85, 90, 95, or 98%) identical amino acid sequences, or Cas12a-inhibiting fragments thereof. For example, exemplary fragments can include at least 20, 30, 40, 50 60, 70, 80, 90, or 100 amino acids of any of the sequences provided herein. In some embodiments, active fragments of naturally-occurring Cas12a-inhibiting proteins can be used, including for example, fragments that are amino or carboxyl-terminus truncations lacking, e.g., 1, 2, 3, 4, 5, 10 or more amino acids compared to the naturally occurring protein. In some embodiments, the polypeptides or Cas12a-inhibiting fragments thereof, in addition to having one of the above-listed sequences, will include other amino acid sequences or other chemical moieties (e.g., detectable labels) at the amino terminus, carboxyl terminus, or both. Additional amino acid sequences can include, but are not limited to, tags, detectable markers, or nuclear localization signal sequences.

As noted in the examples, a number of the Cas12a-inhibiting polypeptides have been shown to inhibit Moraxella bovoculi Cas12a (MbCas 12a). It is believed and expected that the Cas12-inhibiting polypeptides described herein will also similarly inhibit other Cas12 proteins. As used herein, a “Cas12-inhibiting polypeptide” is a protein that inhibits function of the Cas12 enzyme in a cell-based assay or a cell-free assay as described below.

In the cell-based assay, Pseudomonas aeruginosa is modified to express MbCas12a plus or minus phage-targeting gRNA (gp23 or gp24) upon induction. The gRNAs are targeting gene 23 or 24 of a particular Pseudomonas aeruginosa phage, JBD30. Bacterial lawns of the modified Pseudomonas aeruginosa expressing a gRNA or a no gRNA control can be infected with serial dilutions of phage and assessed for plaque formation. Co-expression of Cas12a and the gRNA results in a reduction of phage titer (e.g., by at least 3 orders of magnitude relative to the no gRNA control). Activity of Cas12a-inhibiting polypeptides can be assayed by introducing the polypeptide into a strain that targets the phage and assessing the restoration of plaque formation frequency, as a measure of Cas12a inhibition. Thus, for example, the presence of an active Cas12a-inhibiting polypeptide should result in more plaques compared to the no-Cas12a-inhibiting polypeptide control, and the number of plaques in the presence of an active Cas12a-inhibiting polypeptide should be closer to the number of plaques in the no gRNA control than to the number of plaques in the control having the phage-targeting gRNA and lacking the Cas12a-inhibiting polypeptide. In this assay, a restoration of plaquing by at least 1 order of magnitude is considered a positive result, and indicative of an active Cas12a-inhibiting polypeptide.

In the cell-free assay, a transcription-translation system is used (e.g., based on E. coli S30 extracts) where two fluorescent reporters (GFP and RFP) are co-expressed with Cas12a and guide RNAs targeting both reporters. Without Cas12a-inhibiting activity, the Cas12a and gRNAs are expressed and target the reporter plasmids, cleaving them and preventing reporter expression. With Cas12a-inhibiting activity, the Cas12a would be inhibited, and the reporters are expressed, producing a fluorescence curve over time as the reaction proceeds.

The Cas12a-inhibiting polypeptides can be generated by any method. For example, in some embodiments the protein can be purified from naturally-occurring sources, synthesized, or more typically can be made by recombinant production in a cell engineered to produce the protein. Exemplary expression systems include various bacterial, yeast, insect, and mammalian expression systems.

The Cas12a-inhibiting proteins as described herein can be fused to one or more fusion partners and/or heterologous amino acids to form a fusion protein. Fusion partner sequences can include, but are not limited to, amino acid tags, non-L (e.g., D-) amino acids or other amino acid mimetics to extend in vivo half-life and/or protease resistance, targeting sequences or other sequences. In some embodiments, functional variants or modified forms of the Cas12a-inhibiting proteins include fusion proteins of a Cas12a-inhibiting protein and one or more fusion domains. Exemplary fusion domains include, but are not limited to, polyhistidine, Glu-Glu, glutathione S transferase (GST), thioredoxin, protein A, protein G, an immunoglobulin heavy chain constant region (Fc), maltose binding protein (MBP), and/or human serum albumin (HSA). A fusion domain or a fragment thereof may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QLAexpress™ system (Qiagen) useful with (HIS6) fusion partners. As another example, a fusion domain may be selected so as to facilitate detection of the Cas12a-inhibiting proteins. Examples of such detection domains include the various fluorescent proteins (e.g., GFP) as well as “epitope tags,” which are usually short peptide sequences for which a specific antibody is available. Epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain embodiments, a Cas12a-inhibiting protein is fused with a domain that stabilizes the Cas12a-inhibiting protein in vivo (a “stabilizer” domain). By “stabilizing” is meant anything that increases serum half-life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. See, e.g., US Patent Publication No. 2014/056879. Likewise, fusions to human serum albumin can confer desirable properties. Other types of fusion domains that may be selected include multimerizing (e.g., dimerizing, tetramerizing) domains and functional domains (that confer an additional biological function, as desired). Fusions may be constructed such that the heterologous peptide is fused at the amino terminus of a Cas12a-inhibiting polypeptide and/or at the carboxyl terminus of a Cas12a-inhibiting polypeptide.

In some embodiments, the Cas12a-inhibiting polypeptides as described herein comprise at least one non-naturally encoded amino acid. In some embodiments, a polypeptide comprises 1, 2, 3, 4, or more unnatural amino acids. Methods of making and introducing a non-naturally-occurring amino acid into a protein are known. See, e.g., U.S. Pat. Nos. 7,083,970; and 7,524,647. The general principles for the production of orthogonal translation systems that are suitable for making proteins that comprise one or more desired unnatural amino acid are known in the art, as are the general methods for producing orthogonal translation systems. For example, see International Publication Numbers WO 2002/086075, entitled “METHODS AND COMPOSITION FOR THE PRODUCTION OF ORTHOGONAL tRNA-AMINOACYL-tRNA SYNTHETASE PAIRS;” WO 2002/085923, entitled “IN VIVO INCORPORATION OF UNNATURAL AMINO ACIDS;” WO 2004/094593, entitled “EXPANDING THE EUKARYOTIC GENETIC CODE;” WO 2005/019415, filed Jul. 7, 2004; WO 2005/007870, filed Jul. 7, 2004; WO 2005/007624, filed Jul. 7, 2004; WO 2006/110182, filed Oct. 27, 2005, entitled “ORTHOGONAL TRANSLATION COMPONENTS FOR THE VIVO INCORPORATION OF UNNATURAL AMINO ACIDS” and WO 2007/103490, filed Mar. 7, 2007, entitled “SYSTEMS FOR THE EXPRESSION OF ORTHOGONAL TRANSLATION COMPONENTS IN EUBACTERIAL HOST CELLS.” For discussion of orthogonal translation systems that incorporate unnatural amino acids, and methods for their production and use, see also, Wang and Schultz, (2005) “Expanding the Genetic Code.” Angewandte Chemie Int Ed 44: 34-66; Xie and Schultz, (2005) “An Expanding Genetic Code.” Methods 36: 227-238; Xie and Schultz, (2005) “Adding Amino Acids to the Genetic Repertoire.” Curr Opinion in Chemical Biology 9: 548-554; and Wang, et al., (2006) “Expanding the Genetic Code.” Annu Rev Biophys Biomol Struct 35: 225-249; Deiters, et al, (2005) “In vivo incorporation of an alkyne into proteins in Escherichia coli.” Bioorganic & Medicinal Chemistry Letters 15:1521-1524; Chin, et al., (2002) “Addition of p-Azido-L-phenylalanine to the Genetic Code of Escherichia coli.” J Am Chem Soc 124: 9026-9027; and International Publication No. W02006/034332, filed on Sep. 20, 2005. Additional details are found in U.S. Pat. Nos. 7,045,337; 7,083,970; 7,238,510; 7,129,333; 7,262,040; 7,183,082; 7,199,222; and 7,217,809.

A non-naturally encoded amino acid is typically any structure having any substituent side chain other than one used in the twenty natural amino acids. Because non-naturally encoded amino acids typically differ from the natural amino acids only in the structure of the side chain, the non-naturally encoded amino acids form amide bonds with other amino acids, including but not limited to, natural or non-naturally encoded, in the same manner in which they are formed in naturally occurring polypeptides. However, the non-naturally encoded amino acids have side chain groups that distinguish them from the natural amino acids. For example, R optionally comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amino group, or the like or any combination thereof. Other non-naturally occurring amino acids of interest that may be suitable for use include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, amino acids comprising biotin or a biotin analog, glycosylated amino acids such as a sugar substituted serine, other carbohydrate modified amino acids, keto-containing amino acids, amino acids comprising polyethylene glycol or polyether, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, amino thioacid containing amino acids, and amino acids comprising one or more toxic moiety.

Another type of modification that can optionally be introduced into the Cas12a-inhibiting proteins (e.g. within the polypeptide chain or at either the N- or C-terminal), e.g., to extend in vivo half-life, is PEGylation or incorporation of long-chain polyethylene glycol polymers (PEG). Introduction of PEG or long chain polymers of PEG increases the effective molecular weight of the present polypeptides, for example, to prevent rapid filtration into the urine. In some embodiments, a Lysine residue in the Cas12a-inhibiting sequence is conjugated to PEG directly or through a linker. Such linker can be, for example, a Glu residue or an acyl residue containing a thiol functional group for linkage to the appropriately modified PEG chain. An alternative method for introducing a PEG chain is to first introduce a Cys residue at the C-terminus or at solvent exposed residues such as replacements for Arg or Lys residues. This Cys residue is then site-specifically attached to a PEG chain containing, for example, a maleimide function. Methods for incorporating PEG or long chain polymers of PEG can include, for example, those described in Veronese, F. M., et al., Drug Disc. Today 10: 1451-8 (2005); Greenwald, R. B., et al., Adv. Drug Deliv. Rev. 55: 217-50 (2003); Roberts, M. J., et al., Adv. Drug Deliv. Rev., 54: 459-76 (2002)), the contents of which are incorporated herein by reference.

Another alternative approach for incorporating PEG or PEG polymers through incorporation of non-natural amino acids (e.g., as described above) can be performed with the present Cas12a-inhibiting polypeptides. This approach utilizes an evolved tRNA/tRNA synthetase pair and is coded in the expression plasmid by the amber suppressor codon (Deiters, A, et al. (2004). Bio-org. Med. Chem. Lett. 14, 5743-5). For example, p-azidophenylalanine can be incorporated into the present polypeptides and then reacted with a PEG polymer having an acetylene moiety in the presence of a reducing agent and copper ions to facilitate an organic reaction known as “Huisgen [3+2]cycloaddition.”

In certain embodiments, specific mutations of Cas12a-inhibiting proteins can be made to alter the glycosylation of the polypeptide. Such mutations may be selected to introduce or eliminate one or more glycosylation sites, including but not limited to, O-linked or N-linked glycosylation sites as recognized by eukaryotic expression systems (native Cas12a-inhibiting proteins are not glycosylated). In certain embodiments, a variant of Cas12a-inhibiting proteins includes a glycosylation variant wherein the number and/or type of glycosylation sites have been altered relative to a naturally-occurring Cas12a-inhibiting protein sequence expressed in a eukaryotic expression system. In certain embodiments, a variant of a polypeptide comprises a greater or a lesser number of N-linked glycosylation sites relative to a native polypeptide. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate this sequence will remove an existing N-linked carbohydrate chain. In certain embodiments, a rearrangement of N-linked carbohydrate chains is provided, wherein one or more N-linked glycosylation sites (typically those that are naturally occurring) are eliminated and one or more new N-linked sites are created.

In some embodiments, the Cas12a-inhibiting polypeptide is contacted with the Cas12a protein in vitro, e.g., outside of or in the absence of a cell. In some embodiments, the Cas12a-inhibiting polypeptides can be introduced into a cell to inhibit Cas12a in that cell. In some embodiments, the cell contains Cas12a protein when the Cas12a-inhibiting polypeptide is introduced into the cell. In other embodiments, the Cas12a-inhibiting polypeptide is introduced into the cell and then Cas12a polypeptide is introduced into the cell.

Introduction of the Cas12a-inhibiting polypeptides into the cell can take different forms. For example, in some embodiments, the Cas12a-inhibiting polypeptides themselves are introduced into the cells. Any method for the introduction of polypeptides into cells can be used. For example, in some embodiments, electroporation, or liposomal or nanoparticle delivery to the cells can be employed. In other embodiments, a polynucleotide encoding a Cas12a-inhibiting polypeptide is introduced into the cell and the Cas12a-inhibiting polypeptide is subsequently expressed in the cell. In some embodiments, the polynucleotide is an RNA. In some embodiments, the polynucleotide is a DNA.

In some embodiments, the Cas12a-inhibiting polypeptide is expressed in the cell from RNA encoded by an expression cassette, wherein the expression cassette comprises a promoter operably linked to a polynucleotide encoding the Cas12a-inhibiting polypeptide. In some embodiments, the promoter is heterologous to the polynucleotide encoding the Cas12a-inhibiting polypeptide. Selection of the promoter will depend on the cell in which it is to be expressed and the desired expression pattern. In some embodiments, promoters are inducible or repressible, such that expression of a nucleic acid operably linked to the promoter can be expressed under selected conditions. In some examples, a promoter is an inducible promoter, such that expression of a nucleic acid operably linked to the promoter is activated or increased.

An inducible promoter may be activated by the presence or absence of a particular molecule, for example, doxycycline, tetracycline, metal ions, alcohol, or steroid compounds. In some embodiments, an inducible promoter is a promoter that is activated by environmental conditions, for example, light or temperature. In further examples, the promoter is a repressible promoter such that expression of a nucleic acid operably linked to the promoter can be reduced to low or undetectable levels, or eliminated. A repressible promoter may be repressed by direct binding of a repressor molecule (such as binding of the trp repressor to the trp operator in the presence of tryptophan). In a particular example, a repressible promoter is a tetracycline repressible promoter. In other examples, a repressible promoter is a promoter that is repressible by environmental conditions, such as hypoxia or exposure to metal ions.

In some embodiments, the polynucleotide encoding the Cas12a-inhibiting polypeptide (e.g., as part of an expression cassette) is delivered to the cell by a vector. For example, in some embodiments, the vector is a viral vector. Exemplary viral vectors can include, but are not limited to, adenoviral vectors, adeno-associated viral (AAV) vectors, and lentiviral vectors.

In some embodiments, the Cas12a-inhibiting polypeptide or a polynucleotide encoding the Cas12a-inhibiting polypeptide is delivered as part of or within a cell delivery system. Various delivery systems are known and can be used to administer a composition of the present disclosure, for example, encapsulation in liposomes, microparticles, microcapsules, or receptor-mediated delivery.

Exemplary liposomal delivery methodologies are described in Metselaar et al., Mini Rev. Med. Chem. 2(4):319-29 (2002); O'Hagen et al., Expert Rev. Vaccines 2(2):269-83 (2003); O'Hagan, Curr. Drug Targets Infjct. Disord. 1(3):273-86 (2001); Zho et al., Biosci Rep. 22(2):355-69 (2002); Chikh et al., Biosci Rep. 22(2):339-53 (2002); Bungener et al., Biosci. Rep. 22(2):323-38 (2002); Park, Biosci Rep. 22(2):267-81 (2002); Ulrich, Biosci. Rep. 22(2):129-50; Lofthouse, Adv. Drug Deliv. Rev. 54(6):863-70 (2002); Zhou et al., J. Inmunmunother. 25(4):289-303 (2002); Singh et al., Pharm Res. 19(6):715-28 (2002); Wong et al., Curr. Med. Chem. 8(9):1123-36 (2001); and Zhou et al., Immunonmethods (3):229-35 (1994).

Exemplary nanoparticle delivery methodologies, including gold, iron oxide, titanium, hydrogel, and calcium phosphate nanoparticle delivery methodologies, are described in Wagner and Bhaduri, Tissue Engineering 18(1): 1-14 (2012) (describing inorganic nanoparticles); Ding et al., Mol Ther e-pub (2014) (describing gold nanoparticles); Zhang et al., Langmuir 30(3):839-45 (2014) (describing titanium dioxide nanoparticles); Xie et al., Curr Pharm Biotechnol 14(10):918-25 (2014) (describing biodegradable calcium phosphate nanoparticles); and Sizovs et al., J Am Chem Soc 136(1):234-40 (2014).

Introduction of a Cas12a-inhibiting polypeptide as described herein into a prokaryotic cell can be achieved by any method used to introduce protein or nuclei acids into a prokaryote. In some embodiments, the Cas12a-inhibiting polypeptide is delivered to the prokaryotic cell by a delivery vector (e.g., a bacteriophage) that delivers a polynucleotide encoding the Cas12a-inhibiting polypeptide. In some embodiments, inhibiting Cas12a in the prokaryote could either help that phage kill the bacterium or help other phages kill it.

A Cas12a-inhibiting polypeptide as described herein can be introduced into any cell that contains, expresses, or is expected to express, Cas12a. Exemplary cells can be prokaryotic or eukaryotic cells. Exemplary prokaryotic cells can include but are not limited to, those used for biotechnological purposes, the production of desired metabolites, E. coli and human pathogens. Examples of such prokaryotic cells can include, for example, Escherichia coli, Pseudomonas sp., Corynebacterium sp., Bacillus subtilis, Streptococcus pneumonia, Pseudomonas aeruginosa, Staphylococcus aureus, Campylobacter jejuni, Francisella novicida, Corynebacterium diphtheria, Enterococcus sp., Listeria monocytogenes, Mycoplasma gallisepticum, Streptococcus sp., or Treponema denticola. Exemplary eukaryotic cells can include, for example, fungal, animal (e.g., mammalian) or plant cells. Exemplary mammalian cells include but are not limited to human, non-human primates. mouse, and rat cells. Cells can be cultured cells or primary cells. Exemplary cell types can include, but are not limited to, induced pluripotent cells, stem cells or progenitor cells, and blood cells, including but not limited to hematopoietic stem cells, T-cells or B-cells.

In some embodiments, the cells are removed from an animal (e.g., a human, optionally in need of genetic repair), and then Cas12a, and optionally guide RNAs, for gene editing are introduced into the cell ex vivo, and a Cas12a-inhibiting polypeptide is introduced into the cell. In some embodiments, the cell(s) is subsequently introduced into the same animal (autologous) or different animal (allogeneic).

In any of the embodiments described herein, a Cas12a polypeptide can be introduced into a cell to allow for Cas12a DNA binding and/or cleaving (and optionally editing), followed by introduction of a Cas12a-inhibiting polypeptides as described herein. This timing of the presence of active Cas12a in the cell can thus be controlled by subsequently supplying Cas12a-inhibiting polypeptides to the cell, thereby inactivating Cas12a. This can be useful, for example, to reduce Cas12a “off-target” effects such that non-targeted chromosomal sequences are bound or altered. By limiting Cas12a activity to a limited “burst” that is ended upon introduction of the Cas12a-inhibiting polypeptide, one can limit off-target effects. In some embodiments, the Cas12a polypeptide and the Cas12a-inhibiting polypeptide are expressed from different inducible promoters, regulated by different inducers. These embodiments allow for first initiating expression of the Cas12a polypeptide, followed later by induction of the Cas12a-inhibiting polypeptide, optionally while removing the inducer of Cas12a expression.

In some embodiments, a Cas12a-inhibiting polypeptide as described herein can be introduced (e.g., administered) to an animal (e.g., a human) or plant or plant cell. This can be used to control in vivo Cas12a activity, for example in situations in which CRISPR/Cas12a gene editing is performed in vivo, or in circumstances in which an individual is exposed to unwanted Cas12a, for example where a bioweapon comprising Cas12a is released.

In some embodiments, the Cas12a-inhibiting polypeptide, or a polynucleotide encoding the Cas12a-inhibiting polypeptide, is administered as a pharmaceutical composition. In some embodiments, the composition comprises a delivery system such as a liposome, nanoparticle or other delivery vehicle as described herein or otherwise known, comprising the Cas12a-inhibiting polypeptide or a polynucleotide encoding the Cas12a-inhibiting polypeptide. The compositions can be administered directly to a mammal (e.g., human) to inhibit Cas12a using any route known in the art, including e.g., by injection (e.g., intravenous, intraperitoneal, subcutaneous, intramuscular, or intrademal), inhalation, transdermal application, rectal administration, or oral administration.

The pharmaceutical compositions may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

### EXAMPLES

The discovery of bacterial CRISPR-Cas systems that prevent infection by bacterial viruses (phages) has opened a new paradigm for bacterial immunity while yielding exciting new tools for targeted genome editing. Although CRISPR-Cas systems have seemingly evolved to target phage for cleavage and destruction, phages have been found to express anti-CRISPR (Acr) proteins that directly inhibit Cas effectors (1, 2). CRISPR-Cas systems are spread widely across the bacterial world, divided into six distinct types (I-VI), but anti-CRISPR proteins have only been discovered for type I and II CRISPR systems (3-5). Given the prevalence and diversity of CRISPR-Cas systems, we hypothesized that anti-CRISPR proteins against other types and sub-types exist.

Anti-CRISPR proteins do not have conserved sequences or structures and only share their relatively small size (˜50-150 amino acids), making de novo prediction of acr function difficult (6). However, distinct acr genes often cluster together in operons with other acr genes and/or adjacent to highly conserved anti-CRISPR associated genes (aca genes) in “acr loci” (7). Previously, Pawluk et al. leveraged genes aca1-3 to find new families of Acr proteins throughout Proteobacteria (8), demonstrating the utility of “guilt-by-association” bioinformatics searches. In this work, we sought to expand the current list of acr and aca genes with the goal of unlocking new anti-CRISPR loci in bacterial species with no homologs of previously identified acr or aca genes.

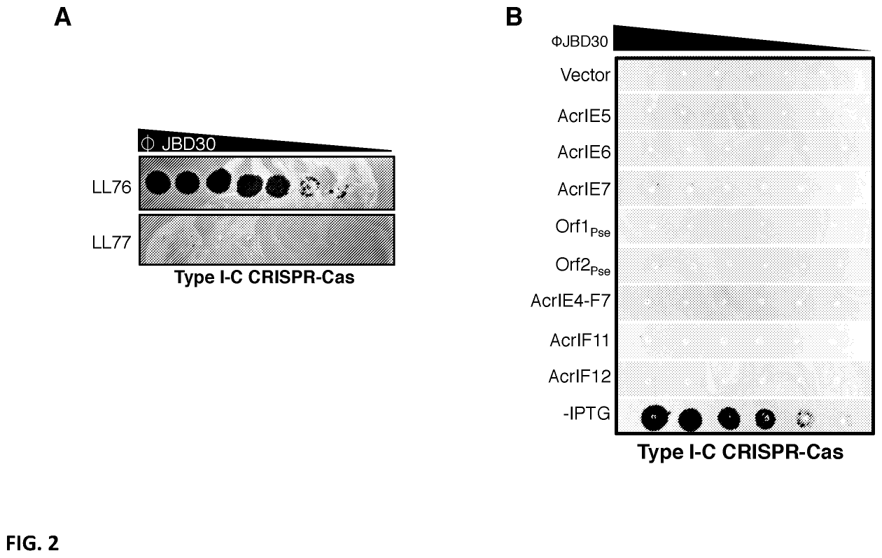

Anti-CRISPRs were first discovered in Pseudomonas aeruginosa, inhibiting Type I-F and I-E CRISPR-Cas systems (1, 9). In addition to type I-E and I-F, P. aeruginosa strains encode a third CRISPR-Cas subtype (type I-C), which lacks known inhibitors (10). In search of novel anti-CRISPRs in Pseudomonas, we established a P. aeruginosa strain where we could assay Type I-C CRISPR-Cas function, expressing a CRISPR RNA (crRNA) targeting phage JBD30 and cas3-cas5-cas7-cas8 under the control of an inducible promoter (FIG. 2A). This system was used in parallel with existing Type I-E (strain PA4386) and I-F (strain PA14) CRISPR-Cas systems to screen for novel anti-CRISPR genes.

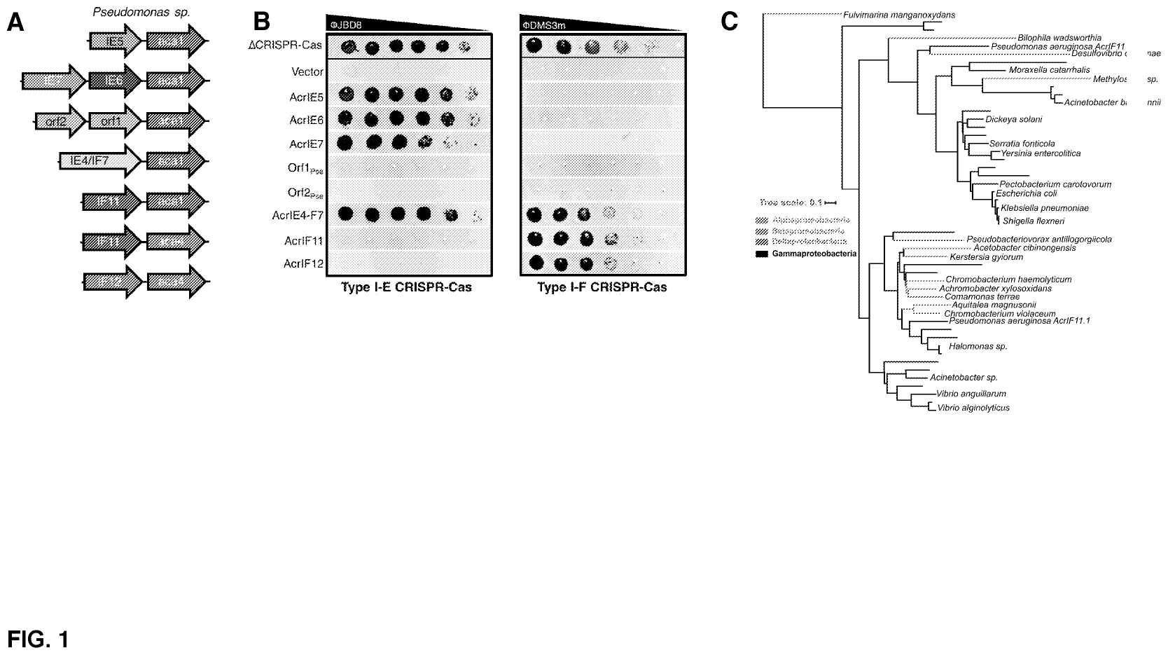

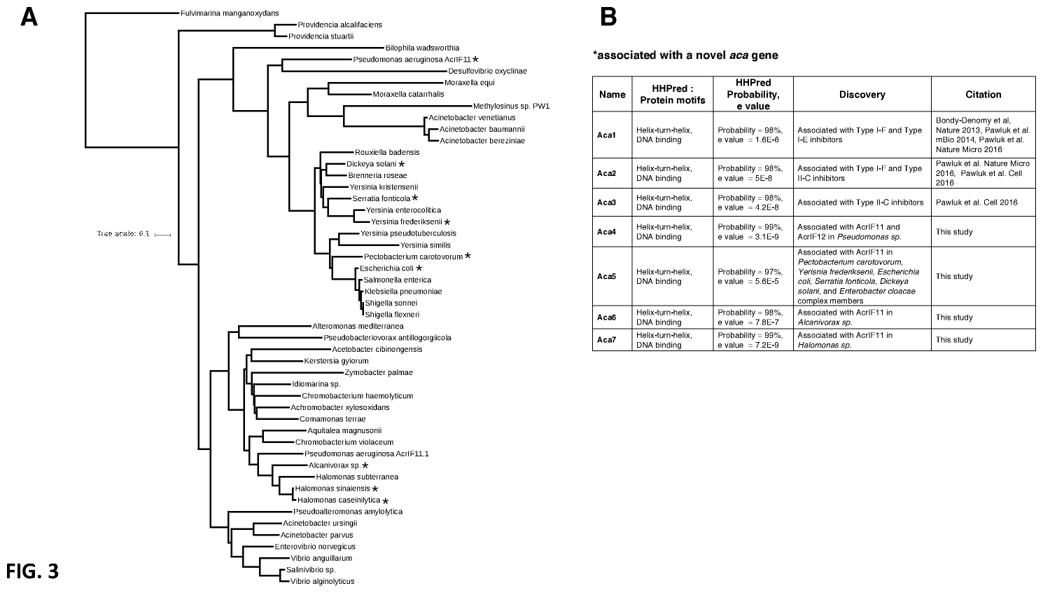

We searched Pseudomonas sp. genomes for homologs of the anti-CRISPR associated gene aca1, and identified 7 genes families upstream of aca1 not previously tested for anti-CRISPR function (FIG. 1A). To test these genes for acr function, we overexpressed them individually in the three Type I CRISPR-Cas immunity model strains. Three genes inhibited the Type I-E CRISPR-Cas system (AcrIE5-7), and one gene inhibited Type I-F CRISPR immunity (AcrIF11) (FIG. 1B). Another gene exhibited dual activity against the I-E and I-F system, and domain analysis demonstrated the gene to be a chimera of previously identified anti-CRISPRs AcrIE4 and AcrIF7 (AcrIE4-F7). None of the genes tested exhibited inhibitory activity against the Type I-C system (FIG. 2B). Excitingly, the Type I-F inhibitor AcrIF11 was commonly represented not only in the P. aeruginosa mobilome but was also present in over 50 species of diverse Proteobacteria (FIG. 1C, FIG. 3A). In many cases, acrIF11 was associated with novel genes with DNA-binding motifs, which we have grouped into 4 families and designated aca4-7 (FIG. 3B). To confirm that these new aca genes can be used to facilitate novel acr discovery, we used aca4 to discover an additional Pseudomonas anti-CRISPR, AcrIF12 (FIG. 1A, 1B).

Given the widespread nature of AcrIF11, we reasoned that guilt-by-association bioinformatics could again be used to nucleate the discovery of new Acr proteins against CRISPR-Cas types for which Acrs are yet to be discovered. We selected the Type V-A CRISPR-Cas12a system (formerly Cpf1), a Class 2 single effector system that has received extensive interest due to its high efficiency editing in human cells, its ability to target sites with T-rich protospacer adjacent motifs (PAMs), and a naturally encoded ribonuclease activity that simplifies multiplex targeting (11-14). However, much less is known about Cas12 biology and there are no known Acr proteins that regulate Cas12a activity. To select an ideal bacterium to search for AcrVA proteins in, we first looked for instances of Cas12a intragenomic “self-targeting”, which describes the co-occurrence of a CRISPR spacer and its target protospacer within the same genome. The existence of self-targeting in viable bacteria indicates potential inactivation of the CRISPR-Cas system, since genome cleavage would result in bacterial death. This strategy was also used previously to discover Type II-A CRISPR-Cas9 inhibitors (4).

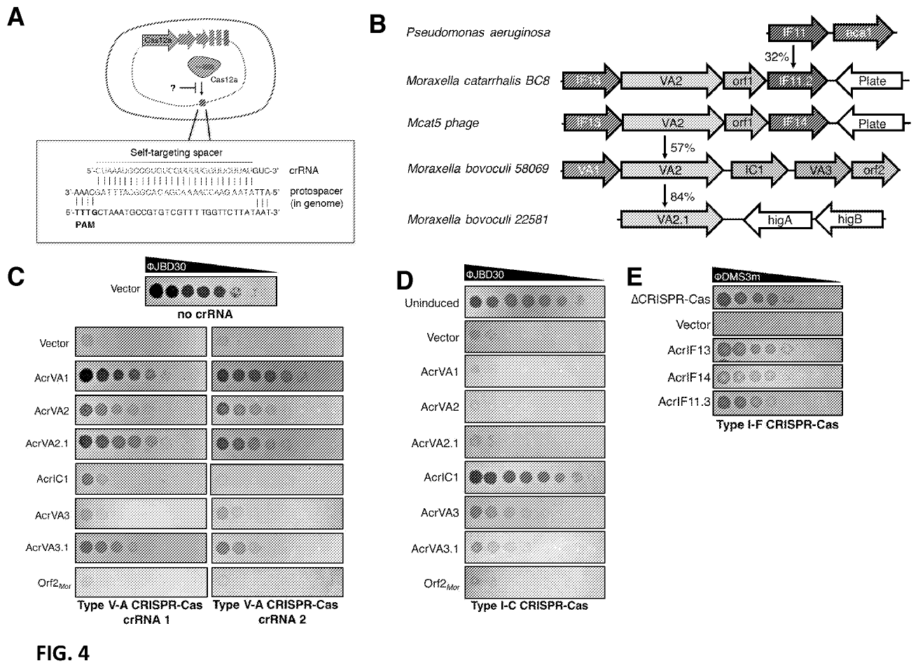

The Gram negative bovine pathogen Moraxella bovoculi (15, 16) was identified as a CRISPR-Cas12a-containing organism (11) where four of the seven genomes featured intragenomic self-targeting (FIG. 4A). Interestingly, the 58069 strain of Moraxella bovoculi also encodes a Type I-C CRISPR-Cas system that also exhibited extensive intragenomic self-targeting. Although no previously described acr or aca genes were present in this strain, an acrIF11 homolog was found in the human pathogen Moraxella catarrhalis, a close relative of M. bovoculi. Interestingly, homologs of neighbors of the acrIF11 gene in M. catarrhalis appeared in the self-targeting M. bovoculi strains, so these genes were selected as candidates acrVA genes (FIG. 4B).

Due to the limited tools available for the genetic manipulation of Moraxella sp., a lab strain of Pseudomonas aeruginosa PAO1 was engineered to express MbCas12a and a crRNA targeting P. aeruginosa phage JBD30. Two distinct crRNAs that target gp23 and gp24 were used, showing strong reduction of titer by >4 orders of magnitude (FIG. 4C). Candidate genes were selected from M. bovoculi self-targeting strains and tested for inhibition of Cas12a, revealing that two genes, AAX09_07405 (now AcrVA1) and AAX09_07410 (AcrVA2), from M. bovoculi 58069 restored phage titers nearly to levels seen with the crRNA-minus control (FIG. 4C). An ortholog of AcrVA2 (AcrVA2.1) with 84% identity was found in the other three self-targeting strains of Moraxella bovoculi and also functioned as an anti-CRISPR (FIG. 4B, 4C). An additional gene from this locus, AAX09_07420 (AcrVA3), and an ortholog with 43% sequence identity, B0181_04965 (AcrVA3.1), encoded by Moraxella caviae CCUG 355, showed mild but reproducible increases in phage titer by one and two orders of magnitude, respectively (FIG. 4C).

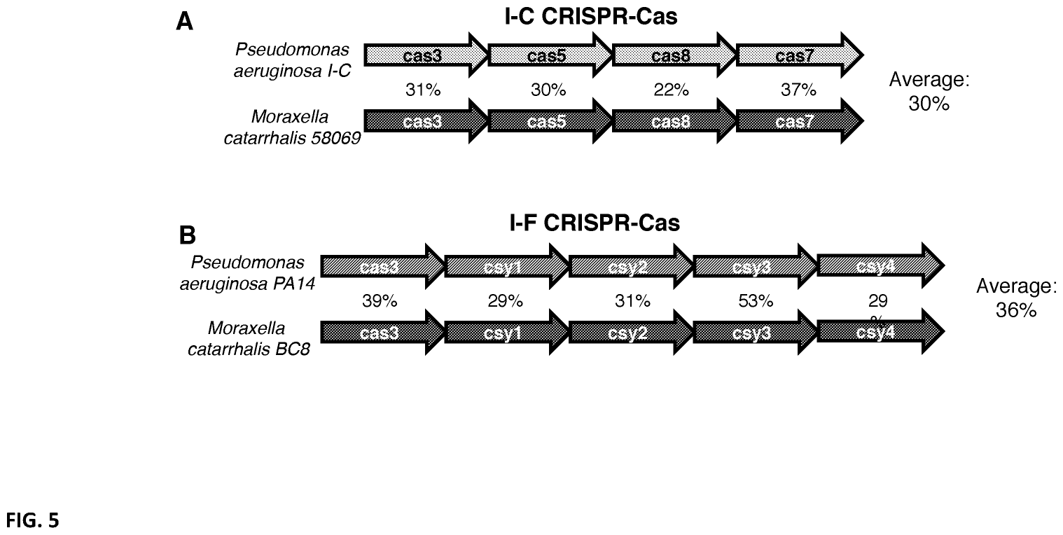

It has been previously shown that acr genes inhibiting distinct subtypes (i.e. acrIE and acrIF genes) cluster together (9), while acr genes that inhibit completely different CRISPR-Cas types have not yet been reported in the same locus. We considered whether the remaining genes in this locus may function as inhibitors of the Type I-C or I-F CRISPR-Cas systems, which are also present in Moraxella. Given the Type I-C self-targeting seen in strain 58069, we tested genes from this strain against the P. aeruginosa I-C system introduced above. Although not identical to the I-C system of M. bovoculi, the four effector proteins (Cas3, Cas5, Cas7, Cas8) share an average of 30% sequence identity (FIG. 5A). Indeed, we found that candidate gene AAX09_07415 (AcrIC1) robustly inhibits the type I-C system (FIG. 4D). Surprisingly, AcrVA3 and AcrVA3.1 also showed partial restoration of phage titer, suggesting that they may inhibit the type I-C as well as type V-A system (FIG. 4D). Bifunctional anti-CRISPR proteins that inhibit type I-E and I-F CRISPR-Cas systems have previously been reported (e.g. AcrIF6) (8); however, this is the first anti-CRISPR protein shown to target different types of CRISPR-Cas systems.

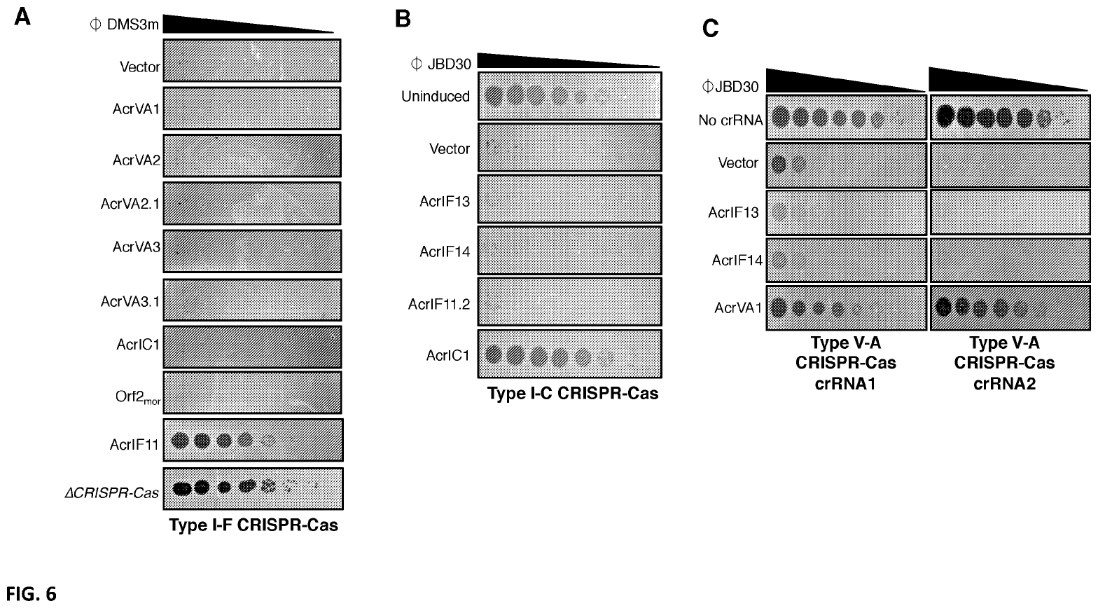

Lastly, this new acr locus was assayed for Type I-F CRISPR-Cas inhibition, which is absent from M. bovoculi but present in M. catarrhalis. As a surrogate host, we used the well-characterized I-F system in the PA14 strain of P. aeruginosa, which naturally expresses the I-F system and a spacer that targets DMS3m phage (17). Although not identical to the I-F system of M. catarrhalis, the five P. aeruginosa effector proteins (Csy1-Csy4, Cas3) share an average of 36% sequence identity (FIG. 5B). None of the candidates within the M. bovoculi acr locus affected targeting by the I-F system (FIG. 6A); however, gene E9U_08483 (AcrIF13) from the Moraxella catarrhalis BC8 prophage restored phage titers nearly to levels seen in the ΔCRISPR-Cas mutant, while E9U_08473 (orf2,nor) had no inhibitory activity (FIG. 4E, FIG. 6). Other prophages of Moraxella catarrhalis were then searched for orthologs of AcrIF11 and AcrVA2 to unlock novel anti-CRISPR loci. A hypothetical protein AK127193 (AcrIF14) was identified in phage Mcat5 at the same position as AcrIF11 in BC8 (FIG. 4B), which also inhibited Type I-F function, but not I-C or V-A (FIG. 4E, FIG. 6B, C). In sum, the combination of using self-targeting to motivate specific strain selection, and the use of an anti-CRISPR “key” AcrIF11, have unlocked seven new acr genes inhibiting Type I-C, I-F, and V-A in Moraxella. Below, we focus on the evolutionary analysis of Type V-A inhibitors, and on their function in mammalian cells.

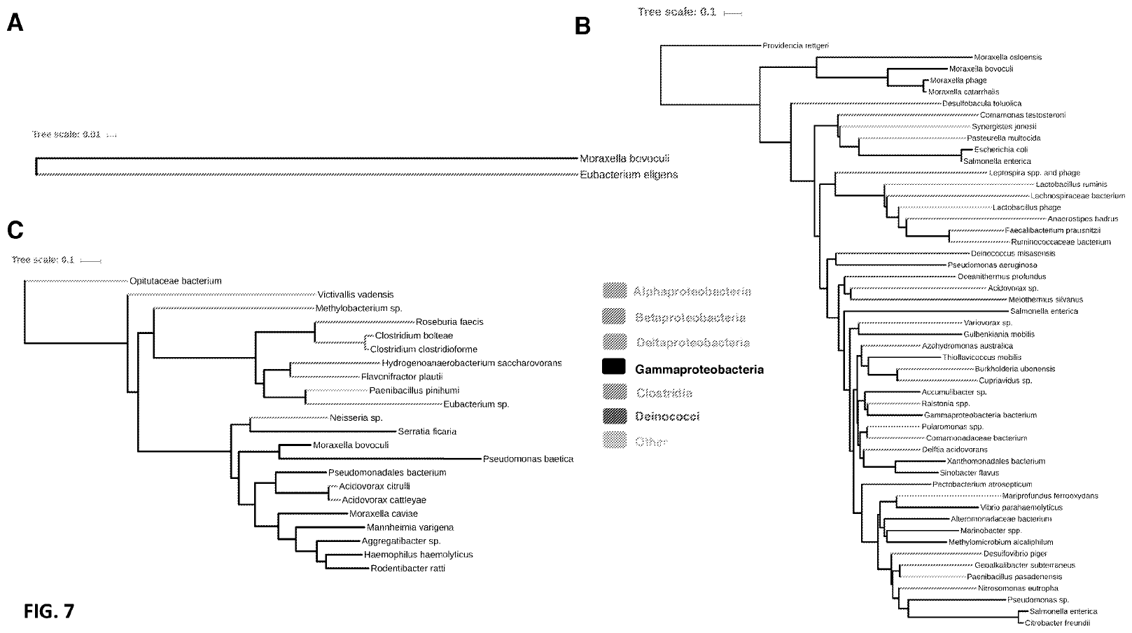

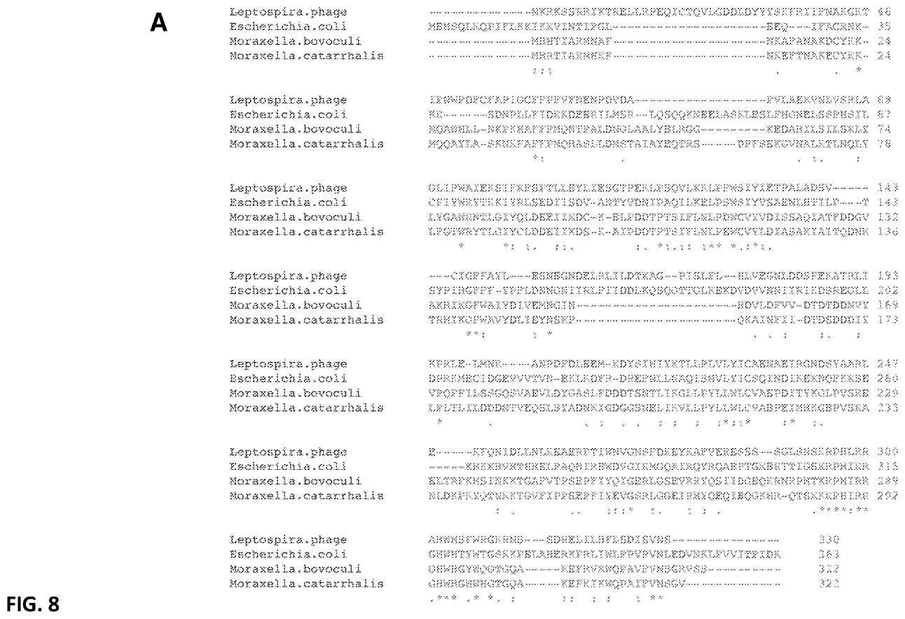

acrVA1 encodes a 170 amino acid protein, found only in Moraxella sp. and Eubacterium eligens (FIG. 7A), both Type V-A CRISPR-Cas-containing organisms. Although AcrVA1 from M. bovoculi strain 58069 is in a region not annotated as a prophage, a prophage was identified 5 genes downstream of this anti-CRISPR locus, with a DUF4102 domain phage integrase 1 gene upstream. We therefore conclude that this novel locus containing inhibitors of both Type V-A and I-C CRISPR-Cas systems are likely within a prophage.

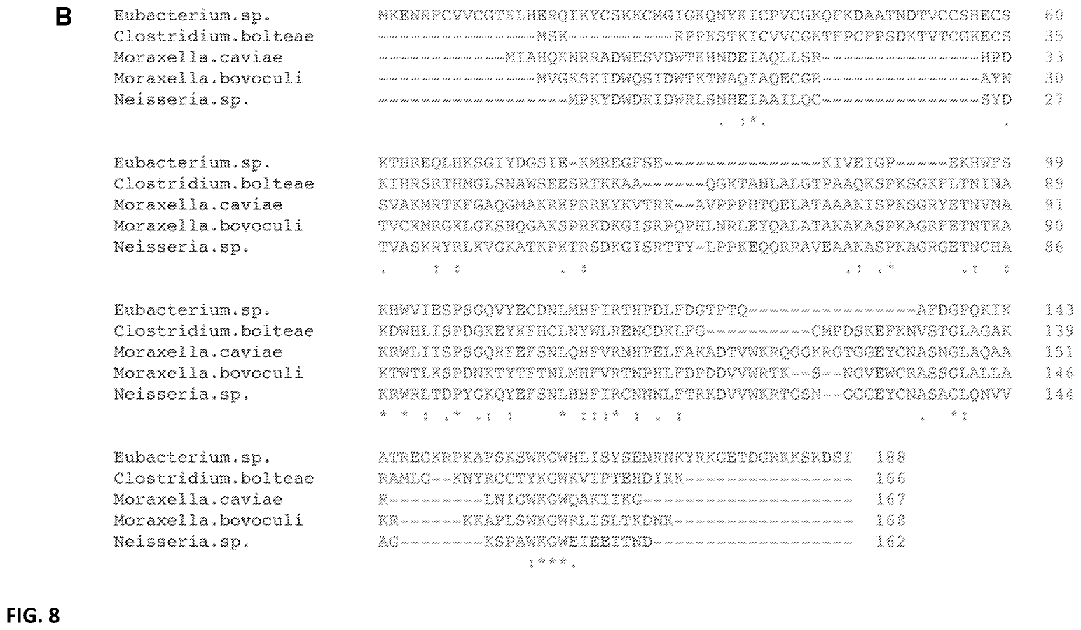

acrVA2 encodes a 322 amino acid protein, the largest Acr protein discovered to date, although it is occasionally seen as two separate proteins (i.e. M. catarrhalis BC1). acrVA2 orthologs are found in many Moraxella species, and broadly across many bacterial phyla (FIG. 7B, FIG. 8), with orthologs present in over 70 different species. acrVA2 orthologs are present in Lachnospiraceae, Leptospira, and Synergistes jonesii (FIG. 7B), all of which contain Type V-A, as well as in Leptospira and Lactobacillus phages. Notably, AcrVA2 is also found in previously described Meat phages (e.g. phage Mcat5, FIG. 4B), where the acr locus also contains novel acrIF genes (acrIF11, acrIF13, and acrIF14) and is found at the far left arm of the annotated prophage genome. Together with the putative prophage described in M. bovoculi 58069 above, these elements are the first examples of acr genes that inhibit distinct CRISPR-Cas types deriving from a single locus. In other isolates, including M. bovoculi 22581, acrVA2.1 is found upstream of the higA-higB toxin-antitoxin pair (FIG. 4B), previously implicated in plasmid addiction, but frequently found in chromosomes (18), as it is here. Although the function of this locus remains to be determined, it is clear that Type V-A CRISPR-Cas inhibitors also occur in non-phage elements. Interestingly, distant orthologs of acrVA2 were also identified on plasmids and conjugative elements in bacteria that lack known Type V-A CRISPR-Cas, such as E. coli. BLASTp searches revealed homology to finQ from E. coli IncI plasmid R62 (28% sequence identity, 41% similarity over 94% of the protein, E value=2×10⁻¹⁵, FIGS. 6-8). Although not well characterized, FinQ is an inhibitor of the F plasmid transfer genes, proposed to cause transcriptional termination of tra genes, thus preventing conjugation (19-21). InterPro analysis did not reveal any conserved motifs or domains in acrVA2, but protein alignments of diverse orthologs from M. bovoculi, M. catarrhalis, Leptospira phage, and E. coli (FinQ) show conservation of a basic 11 amino acid stretch in the C-terminal portion. AcrVA2 is the first Acr protein with a previously characterized ortholog, providing a potential evolutionary trajectory (FIGS. 7B, 8).

acrVA3 encodes a 168 amino acid protein and is also widespread, being distributed throughout different classes of proteobacteria (FIG. 7C). Among the many homologs found in diverse microbes, one homolog in Neisseria stood out, due to the previous discovery of acrIIC genes in this organism (5). While acrVA3 has no detectable homology to the Neisseria acrIIC genes, the acrVA3 homlog in Neisseria is flanked by a putative DNA-binding protein, homologous to the previously identified aca3 (anti-CRISPR associated gene 3, WP_049360086, 51% sequence identity, E value=2×10⁻²²). aca3 is adjacent to acrIIC1-3 in different Neisseria genomes, and its association with acrVA3 suggests that this gene may perform anti-CRISPR functions in Neisseria. Orthologs of acrVA3 are also present in Eubacterium and Clostridium species, which encode Type V-A CRISPR-Cas.

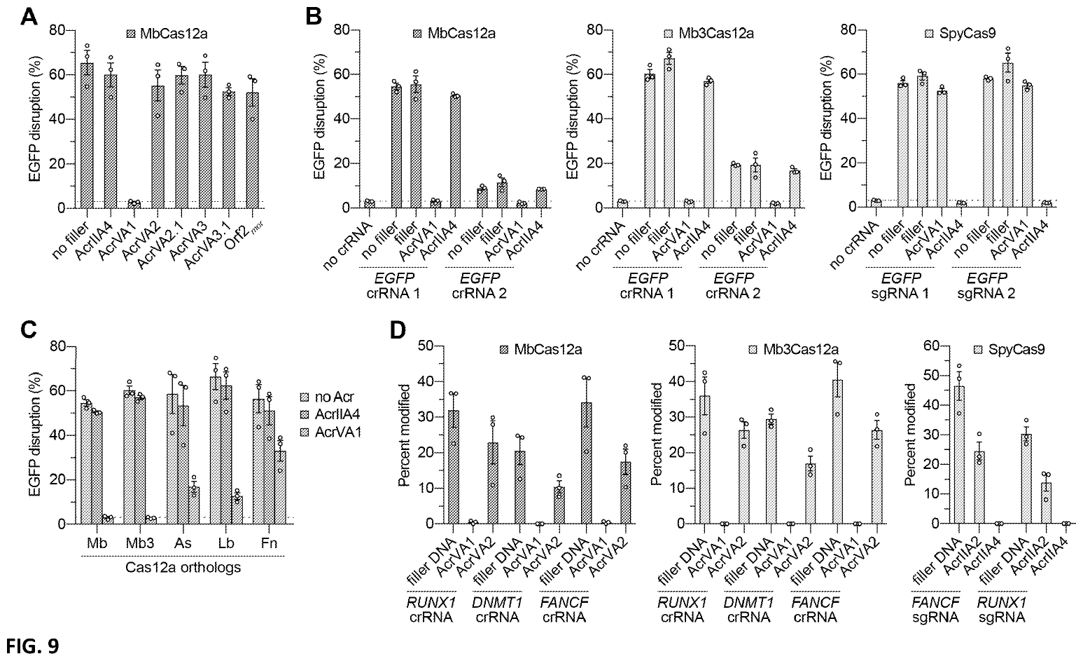

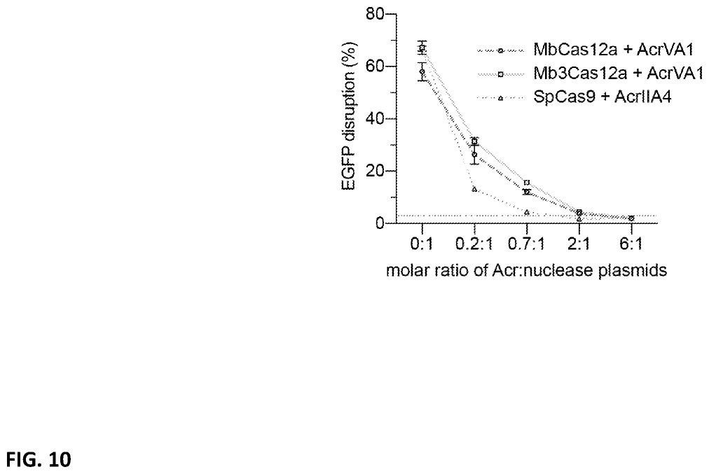

Given the inhibitory effect of acrVA1-3.1 on MbCas12a in bacteria, we sought to determine whether any of these AcrVA proteins could repress MbCas12a activity in human cells. Human U2-OS-EGFP cells (22) were co-transfected with a MbCas12a nuclease expression plasmid, an EGFP-targeting crRNA plasmid, and an anti-CRISPR expression plasmid. The U2-OS-EGFP cell line contains a single integrated copy of EGFP reporter gene that is constitutively expressed. Cells were then harvested and analyzed for EGFP fluorescence using flow cytometry. As expected, co-transfection of the MbCas12a nuclease and crRNA expression plasmid in a control experiment resulted in ˜60-70% disruption of EGFP expression relative to background (indicated by the red dashed line). Upon co-transfection with acrVA1, however, EGFP disruption was reduced to background levels, suggesting AcrVA1-mediated inhibition MbCas12a EGFP targeting (FIG. 9A). The activities of the other AcrVA proteins and orthologs were also tested but did not reveal substantial inhibition of MbCas12a-mediated EGFP disruption (FIG. 9A). To determine whether AcrVA1 could inhibit the nuclease activity of another Cas12a ortholog, Mb3Cas12, we also examined its activity in human cells (FIG. 9B). Furthermore, we performed similar control experiments with SpyCas9 and an AcrIIA4 expression plasmid that has been previously been shown to inhibit SpyCas9 activity (4) but was not expected to inhibit Cas12a (FIG. 9B). To ensure consistent quantities of DNA in transfections, a “filler” control plasmid was used in lieu of anti-CRISPR plasmid. As expected, AcrIIA4 inhibited SpyCas9-mediated disruption of EGFP to background levels but had no effect on disruption by MbCas12a or Mb3Cas12a (FIG. 9B). Similarly, AcrVA1 completely decreased targeting by MbCas12a and Mb3Cas12a, but had no apparent effect on SpyCas9 (FIG. 9B). Experiments titrating the Acr plasmid relative to the nuclease expression plasmid revealed comparable dose-responses to inhibition between MbCas12a or Mb3Cas12a with AcrVA1 and SpyCas9 with AcrIIA4 (FIG. 10).

Given the robust effect of AcrVA1 on MbCas12a, we examined whether AcrVA1 could inhibit the activities of other commonly used Cas12a orthologs including AsCas12a, LbCas12a, and FnCas12a (11, 23). We observed potent inhibition of AsCas12a and LbCas12a (though less complete compared to MbCas12a) in the presence of AcrVA1, and more modest inhibition of FnCas12a (FIG. 9C).

Next, to determine whether AcrVA1 could inhibit Cas12a-mediated modification of endogenous loci in human cells, U2-OS cells were co-transfected with nuclease and anti-CRISPR expression plasmids, along with plasmids that express crRNAs targeted to sites in endogenous genes (RUNX1, DNMT1, or FANCF). Genomic DNA was then extracted and assessed for modification by T7 endonuclease I (T7E1) assay. As before, we found that AcrVA1 completely inhibited disruption by MbCas12a and Mb3Cas12a but not SpyCas9 (FIG. 9D). Interestingly, we now observed modest inhibition of the activities of MbCas12a and Mb3Cas12a by AcrVA2 in this assay. We suspect that the discrepant results with AcrVA2 between the EGFP disruption and endogenous targeting assays may be due to differences in the kinetics of modification detection in these assays.

Here, we report the discovery of a broadly distributed type I-F Acr protein (AcrIF11), which served as a marker for novel acr loci in Moraxella, leading to the first type V-A and I-C CRISPR-Cas inhibitors. Our findings show that mobile genetic elements can tolerate bacteria with more than one CRISPR-Cas type by possessing multiple Acr proteins in the same locus, which may explain how phages and other MGEs are able to propagate and persist effectively under this pressure. The strategy described herein enabled the identification of novel anti-CRISPR proteins, one of which is able to potently inhibit Cas12a nucleases used in gene editing, for which no anti-CRISPR proteins have previously been found.

### REFERENCES CITED

1. J. Bondy-Denomy, A. Pawluk, K. L. Maxwell, A. R. Davidson, Bacteriophage genes that inactivate the CRISPR/Cas bacterial immune system. Nature. 493, 429-432 (2013).
2. J. Bondy-Denomy et al., Multiple mechanisms for CRISPR-Cas inhibition by anti-CRISPR proteins. Nature. 526, 136-139 (2015).
3. E. V. Koonin, K. S. Makarova, F. Zhang, Diversity, classification and evolution of CRISPR-Cas systems. Curr. Opin. Microbiol. 37, 67-78 (2017).
4. B. J. Rauch et al., Inhibition of CRISPR-Cas9 with Bacteriophage Proteins. Cell. 168, 150-158.e10 (2017).
5. A. Pawluk et al., Naturally Occurring Off-Switches for CRISPR-Cas9. Cell. 167, 1829-1838.e9 (2016).
6. A. L. Borges, A. R. Davidson, J. Bondy-Denomy, The Discovery, Mechanisms, and Evolutionary Impact of Anti-CRISPRs. Annu Rev Virol. 4, 37-59 (2017).
7. A. Pawluk, A. R. Davidson, K. L. Maxwell, Anti-CRISPR: discovery, mechanism and function. Nat. Rev. Microbiol. 16, 12-17 (2018).
8. A. Pawluk et al., Inactivation of CRISPR-Cas systems by anti-CRISPR proteins in diverse bacterial species. Nat. Microbiol. 1, 16085 (2016).
9. A. Pawluk, J. Bondy-Denomy, V. H. W. Cheung, K. L. Maxwell, A. R. Davidson, A new group of phage anti-CRISPR genes inhibits the type I-E CRISPR-Cas system of Pseudomonas aeruginosa. MBio. 5, e00896-e00896-14 (2014).
10. A. van Belkum et al., Phylogenetic Distribution of CRISPR-Cas Systems in Antibiotic-Resistant Pseudomonas aeruginosa. MBio. 6, e01796-15 (2015).
11. B. Zetsche et al., Cpf1 is a single RNA-guided endonuclease of a class 2 CRISPR-Cas system. Cell. 163, 759-771 (2015).
12. B. Zetsche et al., Multiplex gene editing by CRISPR-Cpf1 using a single crRNA array. Nat. Biotechnol. 35, 31-34 (2017).
13. I. Fonfara, H. Richter, M. Bratovi\grave{c}, A. Le Rhun, E. Charpentier, The CRISPR-associated DNA-cleaving enzyme Cpf1 also processes precursor CRISPR RNA. Nature. 532, 517-521 (2016).
14. B. P. Kleinstiver et al., Genome-wide specificities of CRISPR-Cas Cpf1 nucleases in human cells. Nat. Biotechnol. 34, 869-874 (2016).
15. J. A. Angelos, P. Q. Spinks, L. M. Ball, L. W. George, Moraxella bovoculi sp. nov., isolated from calves with infectious bovine keratoconjunctivitis. Int. J. Syst. Evol. Microbiol. 57, 789-795 (2007).
16. A. M. Dickey et al., Large genomic differences between Moraxella bovoculi isolates acquired from the eyes of cattle with infectious bovine keratoconjunctivitis versus the deep nasopharynx of asymptomatic cattle. Vet. Res. 47, 31 (2016).
17. K. C. Cady, J. Bondy-Denomy, G. E. Heussler, A. R. Davidson, G. A. O'Toole, The CRISPR/Cas adaptive immune system of Pseudomonas aeruginosa mediates resistance to naturally occurring and engineered phages. J. Bacteriol. 194, 5728-5738 (2012).
18. T. L. Wood, T. K. Wood, The HigB/HigA toxin/antitoxin system of Pseudomonas aeruginosa influences the virulence factors pyochelin, pyocyanin, and biofilm formation. Microbiologyopen. 5, 499-511 (2016).
19. M. J. Gasson, N. S. Willetts, Further characterization of the F fertility inhibition systems of “unusual” Fin+ plasmids. J. Bacteriol. 131, 413-420 (1977).
20. L. M. Ham, R. Skurray, Molecular analysis and nucleotide sequence of finQ, a transcriptional inhibitor of the F plasmid transfer genes. Mol. Gen. Genet. 216, 99-105 (1989).
21. D. Gaffney, R. Skurray, N. Willetts, Regulation of the F conjugation genes studied by hybridization and tra-lacZ fusion. J. Mol. Biol. 168, 103-122 (1983).
22. D. Reyon et al., FLASH assembly of TALENs for high-throughput genome editing. Nat. Biotechnol. 30, 460-465 (2012).
23. B. Zetsche et al., A Survey of Genome Editing Activity for 16 Cpf1 orthologs (2017), doi:10.1101/134015.
24. J. Söding, A. Biegert, A. N. Lupas, The HHpred interactive server for protein homology detection and structure prediction. Nucleic Acids Res. 33, W244-8 (2005).

TABLE 1
A table of previously discovered aca genes (aca1-3) and novel aca genes found in this study (aca4-7).

| | | HHPred | | |
|---|---|---|---|---|
| | HHPred: | Probability, | | |
| Name | Protein motifs | e value | Discovery | Citation |
| Aca1 | Helix-turn-helix, | Probability = 98%, | Associated with Type I-F and Type | Bondy-Denomy et al, |
| | DNA binding | e value = 1.6E−6 | I-E inhibitors | Nature 2013, Pawluk et al. |
| | | | | mBio 2014, Pawluk et al. |
| | | | | Nature Micro 2016 |
| Aca2 | Helix-turn-helix, | Probability = 98%, | Associated with Type I-F and Type | Pawluk et al. Nature Micro |
| | DNA binding | e value = 5E−8 | II-C inhibitors | 2016, Pawluk et al. Cell |
| | | | | 2016 |
| Aca3 | Helix-turn-helix, | Probability = 98%, | Associated with Type II-C inhibitors | Pawluk et al. Cell 2016 |
| | DNA binding | e value = 4.2E−8 | | |
| Aca4 | Helix-turn-helix, | Probability = 99%, | Associated with AcrIF11 and | This study |
| | DNA binding | e value = 3.1E−9 | AcrIF12 in Pseudomonas sp. | |
| Aca5 | Helix-turn-helix, | Probability = 97%, | Associated with AcrIF11 in | This study |
| | DNA binding | e value = 5.6E−5 | Pectobacterium carotovorum, | |
| | | | Yerisnia frederiksenii, Escherichia | |
| | | | coli, Serratia fonticola, | |
| | | | Dickeya solani, and Enterobacter cloacae | |
| | | | complex members | |
| Aca6 | Helix-turn-helix, | Probability = 98%, | Associated with AcrIF11 in | This study |
| | DNA binding | e value = 7.8E−7 | Alcanivorax sp. | |
| Aca7 | Helix-turn-helix, | Probability = 99%, | Associated with AcrIF11 in | This study |
| | DNA binding | e value = 7.2E−9 | Halomonas sp. | |

All aca proteins are predicted with high confidence to contain helix-turn-helix motifs as predicted by HHPred (24).

TABLE 2
Protein sequences and accession numbers of certain anti-CRISPR proteins
found in this study.

| | | | SEQ |
|---|---|---|---|
| Name | Accession | Protein Sequences | ID NO. |
| AcrIE4-F7 | WP_064584002.1 | MSTQYTYQQIAEDFRLWSEYVDTAGEMSKDEFNSLSTED | 10 |
| | | KVRLQVEAFGEEKSPKFSTKVTTKPDFDGFQFYIEAGRDF | |
| | | DGDAYTEAYGVAVPTNIAARIQAQAAELNAGEWLLVEHE | |
| | | A | |
| AcrIE5 | WP_074973300.1 | MSNDRNGIINQIIDYTGTDRDHAERIYEELRADDRIYFDDS | 40 |
| | | VGLDRQGLLIREDVDLMAVAAEIE | |
| AcrIE6 | WP_087937214.1 | MNNDTEVLEQQIKAFELLADELKDRLPTLEILSPMYTAVM | 41 |
| | | VTYDLIGKQLASRRAELIEILEEQYPGHAADLSIKNLCP | |
| AcrIE7 | WP_087937215.1 | MIGSEKQVNWAKSIIEKEVEAWEAIGVDVREVAAFLRSIS | 42 |
| | | DARVIIDNRNLIHFQSSGISYSLESSPLNSPIFLRRFSACSVG | |
| | | FEEIPTALQRIRSVYTAKLLEDE | |
| AcrIF11 | WP_038819808.1 | MSMELFHGSYEEISEIRDSGVFGGLFGAHEKETALSHGETL | 43 |
| | | HRIISPLPLTDYALNYEIESAWEVALDVAGGDENVAEAIM | |
| | | AKACESDSNDGWELQRLRGVLAVRLGYTSVEMEDEHGT | |
| | | TWLCLPGCTVEKI | |
| AcrIF11.2 | EGE18857.1 | MTTLYHGSHENTAPVIKIGFAAFLPADNVFDGIFANGDKN | 44 |
| | | VARSHGDFIYAYEVDSIATNDDLDCDEAIQIIAKELYIDEET | |
| | | AAPIAEAVAYEESLAEFEEHIMPRSCGDCADFGWEMQRLR | |
| | | GVIARKLGFDAVECVDEHGVSHLIVNANIRGSIA | |
| AcrIF12 | ABR13388.1 | MAYEKTWHRDYAAESLKRAETSRWTQDANLEWTQLALE | 45 |
| | | CAQVVHLARQVGEELGNEKIIGIADTVLSTIEAHSQATYRR | |
| | | PCYKRITTAQTHLLAVTLLERFGSARRVANAVWQLTDDEI | |
| | | DQAKA | |
| AcrIF13 | EGE18854.1 | MKLLNIKINEFAVTANTEAGDELYLQLPHTPDSQHSINHEP | 46 |
| | | LDDDDFVKEVQEICDEYFGKGDRTLARLSYAGGQAYDSY | |
| | | TEEDGVYTTNTGDQFVEHSYADYYNVEVYCKADLV | |
| AcrIF14 | AKI27193.1 | MKKIEMIEISQNRQNLTAFLHISEIKAINAKLADGVDVDKK | 47 |
| | | SFDEICSIVLEQYQAKQISNKQASEIFETLAKANKSFKIEKF | |
| | | RCSHGYNEIYKYSPDHEAYLFYCKGGQGQLNKLIAENGRF | |
| | | M | |
| Orf1(Pse) | SDJ61947.1 | MGVVVVLIIRLKARWSLHLERKLGEAGKAGIWEFHRSESS | 48 |
| | | YTTDGRTTFRNAALRPAEPKEGQTVEVFICSDSREPEEQW | |
| | | RAVGEGVARYE | |
| Orf2(Pse) | WP_084336955.1 | MLSVLFFWLYFYALFFIRFASSNKRARGRGMQRPALVSIA | 49 |
| | | LEWGMRRELMSRSFTTRIDHLQEVSRLGRGVARLRLGHS | |
| | | GRNLMPLILERRDGTGLTLKLDPKADPDEALRQLARGGIH | |
| | | VRVYSKYGERMRVVVDAPQAISILRDELVDRE | |
| Aca4 | ABR13385.1 | MTEEQFSALAELMRLRGGPGEDAARLVLVNGLKPTDAAR | 50 |
| | | KTGITPQAVNKTLSSCRRGIELAKRVFT | |
| AcrIC1 | AKG19229.1 | MNNLKKTAITHDGVFAYKNTETVIGSVGRNDIVMAIDAT | 51 |
| | | HGEFNDKNFIIYADTNGNPIYLGYAYLDDNNDAHIDLAVG | |
| | | ACNEDDDFDEKEIHEMIAEQMELAKRYQELGDTVHGTTR | |
| | | LAFDDDGYMTVRLDQQAYPDYRPENDDKHIMWRALALT | |
| | | ATGKELEVFWLVEDYEDEEVNSWDFDIADDWREL | |
| Orf1(Mor) | EGE18856.1 | MSKNKTPDYVLRANANYRKKHTTNKSLQLHNEKDADIIQ | 52 |
| | | ALQNETKSFNALMKDILRNHYNLNQNQ | |
| Orf2(Mor) | AKG19231.1 | MNNPKTPEYTRKAIRAYEKNLVRKSVTFDVRKDDDMELL | 53 |
| | | KMIEQDGRTFAQIARTALLEHLQK | |
| AcrVA1 | AKG19227.1 | MYEAKERYAKKKMQENTKIDTLTDEQHDALAQLCAFRH | 5 |
| | | KFHSNKDSLFLSESAFSGEFSFEMQSDENSKLREVGLPTIE | |
| | | WSFYDNSHIPDDSFREWFNFANYSELSETIQEQGLELDLD | |
| | | DDETYELVYDELYTEAMGEYEELNQDIEKYLRRIDEEHGT | |
| | | QYCPTGFARLR | |
| AcrVA2 | AKG19228.1 | MHHTIARMNAFNKAFANAKDCYKKMQAWHLLNKPKHA | 6 |
| | | FFPMQNTPALDNGLAALYELRGGKEDAHILSILSRLYLYG | |
| | | AWRNTLGIYQLDEEIIKDCKELPDDTPTSIFLNLPDWCVYV | |
| | | DISSAQIATFDDGVAKHIKGFWAIYDIVEMNGINHDVLDF | |
| | | VVDTDTDDNVYVPQPFILSSGQSVAEVLDYGASLFDDDTS | |
| | | NTLIKGLLPYLLWLCVAEPDITYKGLPVSREELTRPKHSIN | |
| | | KKTGAFVTPSEPFIYQIGERLGSEVRRYQSIIDGEQKRNRPH | |
| | | TKRPHIRRGHWHGWQGTGQAKEFRVRWQPAVFVNSGR | |
| | | VSS | |
| AcrVA2.1 | AKG12143.1 | MHHTIARMNAFNKAFGNAKDCYKKMQAWHLNNKPKHIF | 7 |
| | | SPLQNTLSLNEGLAALYELHGGKEDEHILSILCCLYLYGT | |
| | | WRNTLGIYQLDEEIIKDCKELPDDTPTSIFLNLPDWCVYVD | |
| | | ISSAKIATIDGGVAKHIKGFWAIYDNIEMHGVNHDVLNFII | |
| | | DTDTDNNIYVPQSLILSSEMSVAESLDYGLTLFGYDESNEL | |
| | | VKGMLPYLLWLCVAEPDITHKGLPVSREELTKPKHGINKK | |
| | | TGAFVTPSEPFIYQIGERLGGEVRRYQSLIDDEKNQNRHHT | |
| | | KRPHIRRGHWHGYWQGTGQAKEFKVRWQPAVFVNSGV | |
| AcrVA3 | AKG19230.1 | MVGKSKIDWQSIDWTKTNAQIAQECGRAYNTVCKMRGK | 8 |
| | | LGKSHQGAKSPRKDKGISRPQPHLNRLEYQALATAKAKA | |
| | | SPKAGRFETNTKAKTWTLKSPDNKTYTFTNLMHFVRTNP | |
| | | HLFDPDDVVWRTKSNGVEWCRASSGLALLAKRKKAPLS | |
| | | WKGWRLISLTKDNK | |
| AcrVA3.1 | OOR90252.1 | MIAHQKNRRADWESVDWTKHNDEIAQLLSRHPDSVAKM | 9 |
| | | RTKFGAQGMAKRKPRRKYKVTRKAVPPPHTQELATAAA | |
| | | KISPKSGRYETNVNAKRWLIISPSGQRFEFSNLQHFVRNHP | |
| | | ELFAKADTVWKRQGGKRGTGGEYCNASNGLAQAARLNI | |
| | | GWKGWQAKIIKG | |
| AcrVA6 | OOR90226.1 | MNKKSISQRVRRINNPKDKLALVQEWVSQRQSDFFSAFEQ | 39 |
| | | LEYAVGVDDLQQIHEAMDKIKDIAIKNYKAMPNIAEAML | |
| | | VSKHYTVDLDEYEQEK | |

TABLE 3
Type V-A self-targeting spacers in Moraxella
bovoculi strains.
List of spacers encoded in the Type V-A CRISPR
array in Moraxella bovoculi that have matching
protospacers (with PAM motif) in the same genome.
58069, 22581, 28389, and 33362 are all strains.
58069
GCTTCAATCTTGGCAAGTGTTTCATCA
AGATAGGCATTTGAAAAAGAATTTATCT
TTCGTCCTTTATACGCACCCCTTGCTT
22581
ATGGTTAATGATGATAACCCAGATTTAAT
TTTAGAAATCACGGATCATTATATATGT
ATATCCATCTACTAACCATCGCAAAAA
ATTGATGTAAACATCGATGGTGTGGTT
ATTGGTTTGTGTAACGGGGAAATTAAG
TCAAAAATGGTAGCATTTGTTAAGAAT
TGCAGGTGGTGAATCAGCGACACATTC
28389
CTAAATGCCGTGTCGTTTTGGTTCTTAT
ATGAAATAGAGCAACAGCAGAACGGTA
ATTGATGTAAACATCGATGGTGTGGTT
33362
CTAAATGCCGTGTCGTTTTGGTTCTTAT

TABLE 4
Type I-C self-targeting spacers in Moraxella
bovoculi 58069.
List of spacers encoded in the Type I-C CRISPR
array that have matching protospacers (with PAM
motif) in the same genome of Moraxella bovoculi
58069.

ACCCCGTTATCTGCCACGGTGGCGTTGGCTTTGT
ACTTCGCAACATTGGCTATCCAAGTAACGCAAAC
AGCCAAGCTGGTTCGGTTGCCCTTGCCTTTGGAT
ATCGGTTTTGCATTCGGCTAAGGATTTGGGTGTA
ATTTTTAAGCACCACGCCATAATCGCCAAACACC
CAAAGACTGCTTTTTAAGCCAATCATAGTAGCTA
CCAACACGCCTAAGACACGATGACTTGTTTTTAG
TATCTCTTCAGCTTGCTCACGCCAACCCGCCTGC
TGGTGAATTTTCTTTTGAGATGCAGTCTGATGAA
TTTTTCTTGATCGATAGACGACTGATTAAACAAG

TABLE 5
Plasmids used for human cell experiments in this study

| plasmid ID | plasmid use | plasmid description | Addgene ID |
|---|---|---|---|
| BPK3079 | U6 promoter crRNA entry vector used for all | pUC19-U6- | 78741 |
| | AsCas12a crRNAs (clone spacer oligos into | AsCas12a_crRNA- | |
| | BsmBI cassette) | BsmBI_cassette | |
| BPK3082 | U6 promoter crRNA entry vector used for all | pUC19-U6- | 78742 |
| | LbCas12a crRNAs (clone spacer oligos into | LbCas12a_crRNA- | |
| | BsmBI cassette) | BsmBI_cassette | |
| BPK4446 | U6 promoter crRNA entry vector used for all | pUC19-U6- | processing |
| | FnCas12a crRNAs (clone spacer oligos into | FnCas12a_crRNA- | |
| | BsmBI cassette) | BsmBI_cassette | |
| BPK4449 | U6 promoter crRNA entry vector used for all | pUC19-U6- | processing |
| | MbCas12a crRNAs (clone spacer oligos into | MbCas12a_crRNA- | |
| | BsmBI cassette) | BsmBI_cassette | |
| SQT1659 | CAG promoter expression plasmid for human | pCAG-hAsCas12a- | 78743 |
| | codon optimized AsCas12a nuclease with C- | NLS(nucleoplasmin)- | |
| | terminal NLS and HA tag | 3xHA | |
| SQT1665 | CAG promoter expression plasmid for human | pCAG-hLbCas12a- | 78744 |
| | codon optimized LbCas12a nuclease with C- | NLS(nucleoplasmin)- | |
| | terminal NLS and HA tag | 3xHA | |
| AAS1472 | CAG promoter expression plasmid for human | pCAG-hFnCas12a- | processing |
| | codon optimized FnCas12a nuclease with C- | NLS(nucleoplasmin)- | |
| | terminal NLS and HA tag | 3xHA | |
| AAS2134 | CAG promoter expression plasmid for human | pCAG-hMbCas12a- | processing |
| | codon optimized MbCas12a nuclease with C- | NLS(nucleoplasmin)- | |
| | terminal NLS and HA tag | 3xHA | |
| RTW2500 | CAG promoter expression plasmid for human | pCAG-hMb3Cas12a- | processing |
| | codon optimized Mb3Cas12a nuclease with C- | NLS(nucleoplasmin)- | |
| | terminal NLS and HA tag | 3xHA | |
| JDS246 | CMV-T7 promoter expression plasmid for human | pCMV-T7-hSpCas9- | 43861 |
| | codon optimized SpyCas9 nuclease with C- | NLS(sv40)-3xFLAG | |
| | terminal NLS and HA tag | | |
| SQT817 | CAG promoter expression plasmid for human | pCAG-hSpCas9- | 53373 |
| | codon optimized SpyCas9 nuclease with C- | NLS(sv40)-3xFLAG | |
| | terminal NLS and HA tag | | |
| BPK5050 | CMV-T7 promoter expression plasmid for human | pCMV-T7-hAcrVA1- | processing |
| | codon optimized AcrVA1 anti-CRISPR protein | NLS(sv40) | |
| | with C-terminal NLS | | |
| AAS2283 | CMV-T7 promoter expression plasmid for human | pCMV-T7-hAcrVA2- | processing |
| | codon optimized AcrVA2 anti-CRISPR protein | NLS(sv40) | |
| | with C-terminal NLS | | |
| BPK5059 | CMV-T7 promoter expression plasmid for human | pCMV-T7-hAcrVA2.1- | processing |
| | codon optimized AcrVA2.1 anti-CRISPR protein | NLS(sv40) | |
| | with C-terminal NLS | | |
| BPK5077 | CMV-T7 promoter expression plasmid for human | pCMV-T7-hAcrVA3- | processing |
| | codon optimized AcrVA3 anti-CRISPR protein | NLS(sv40) | |
| | with C-terminal NLS | | |
| RTW2624 | CMV-T7 promoter expression plasmid for human | pCMV-T7-hAcrVA3.1- | processing |
| | codon optimized AcrVA3.1 anti-CRISPR protein | NLS(sv40) | |
| | with C-terminal NLS | | |
| BPK5095 | CMV-T7 promoter expression plasmid for human | pCMV-T7-hOrf2mor- | processing |
| | codon optimized Orf2mor anti-CRISPR protein | NLS(sv40) | |
| | with C-terminal NLS | | |
| pJH373 | CMV promoter expression plasmid for human | pCMV-hAcrIIA2 | 86840 |
| | codon optimized AcrIIA2 anti-CRISPR protein | | |
| pJH376 | CMV promoter expression plasmid for human | pCMV-hAcrIIA4 | 86842 |
| | codon optimized AcrIIA4 anti-CRISPR protein | | |

### Materials and Methods

### Bacterial Strains and Growth Conditions

Pseudomonas aeruginosa strains UCBPP-PA14 (PA14) and PAO1 were used in this study. The strains were grown at 37° C. in lysogeny broth (LB) agar or liquid medium, which was supplemented with 50 μg ml⁻¹ gentamicin, 30 μg ml⁻¹ tetracycline, or 250 μg ml⁻¹ carbenicillin as needed to retain plasmids or other selectable markers.

### Phage Isolation

Phage lysates were generated by mixing 10 μl phage lysate with 150 μl overnight culture of P. aeruginosa and pre-adsorbing for 15 min at 37° C. The resulting mixture was then added to molten 0.7% top agar and plated on 1% LB agar overnight at 30° C. or 37° C. The phage plaques were harvested in SM buffer, centrifuged to pellet bacteria, treated with chloroform, and stored at 4° C.

### Bacterial Transformations

Transformations of P. aeruginosa strains were performed using standard electroporation protocols. Briefly, one mL of overnight culture was washed twice in 300 mM sucrose and concentrated tenfold. The resulting competent cells were transformed with 20-200 ng plasmid, incubated in antibiotic-free LB for 1 hr at 37° C., plated on LB agar with selective media, and grown overnight at 37° C. Bacterial transformations for cloning were performed using E. coli DH5a (NEB) and E. coli Stellar competent cells (Takara) according to the manufacturer's instructions.

### Discovery of Novel Acr Genes Using Bioinformatics

All bacterial genome sequences used in this study were downloaded from NCBI. BLASTp was used to search the nonredundant protein database for Aca1 homologs (accession: YP_007392343) in Pseudomonas sp. (taxid: 286). Individual genomes encoding an Aca1 homolog were then manually surveyed for aca1 associated genes. This approach was extended to discover the Aca4 (WP_034011523.1) associated anti-CRISPR AcrIF12. tBLASTn searches to identify orthologs of VA2 in self-targeting Moraxella bovoculi strains were performed using the protein sequence in Moraxella catarrhalis BC8 strain (EGE18855.1) as the query and Moraxella bovoculi genome accessions as the subject (accessions: 58069 genome, CP011374.1; 58069 plasmid, CP011375.1; 22581, CP011376.1; 33362, CP011379.1; 28389, CP011378.1). Other searches for orthologs in Moraxella sp. were performed using BLASTp.

Discovery of Novel Anti-CRISPR Associated (aca) Gene Families

Genomes with homologs of AcrIF11 were manually examined for novel anti-CRISPR associated (aca) genes. A gene was designated as an aca if it fit the following criteria: I) directly downstream of an AcrIF11 homolog in the same orientation, II) a non-identical homolog of this gene exists in the same orientation relative to a non-identical homolog of AcrIF11, and III) predicted in high confidence to contain a DNA-binding domain based on structural prediction using HHPred (probability >90%, E<0.0005) (I). Genes that fit these three criteria were then grouped into sequence families, requiring that a given gene have >40% sequence identity to at least one member of the family for family membership.

Type I-C CRISPR-Cas Expression in Pseudomonas aeruginosa

Reconstitution of the Type I-C system from a P. aeruginosa isolate in the Bondy-Denomy lab into PAO1 was achieved by amplifying the four effector cas genes (cas3-5-8-7) from genomic DNA by PCR and cloning the resulting fragment into the integrative, IPTG-inducible pUC18T-mini-Tn7T-LAC plasmid to generate the pJW31 vector. This plasmid was then electroporated into PAO1 and chromosomal integration was selected for using 50 μg ml⁻¹ gentamicin. After chromosomal integration of the insert was confirmed, the gentamicin selectable marker was removed using flippase-mediated excision at the flippase recognition target (FRT) sites of the construct. CRISPR RNAs (crRNAs) consisting of a spacer that targets JBD30 phage and two flanking repeats were cloned into the mini-CTX2 (AF140577) vector, and the resulting vector was electroporated into PAO1 tn7::pJW31. Stable integration of the vector at the attB site was selected for using 30 μg ml⁻¹ tetracycline. Targeting was confirmed using phage challenge assays, as described in the “bacteriophage plaque assays” section.

Type V-A CRISPR-Cas Expression in Pseudomonas aeruginosa

Human codon-optimized MbCas12a (Moraxella bovoculi 237) was amplified from the pTE4495 plasmid (Addgene #80338) by PCR and cloned into pTN7C130, a mini-Tn7 vector that integrates into the attTn7 site of P. aeruginosa. The pTN7C130 vector expresses MbCas12a off the araBAD promoter upon arabinose induction and contains a gentamicin selectable marker. The resulting construct, pTN7C130-MbCas12a, was used to transform the PAO1 strain of P. aeruginosa, and stable integration of the vector was selected for using 50 μg ml⁻¹ gentamicin and confirmed by PCR. After integration, flippase was used to excise the gentamicin selectable marker from the flippase recognition target (FRT) sites of the construct.

CRISPR RNAs (crRNAs) for MbCas12a were generated by designing oligonucleotides with spacers that target gp23 and gp24 in JBD30 phage flanked by two direct repeats of the MbCas12a crRNA (2). The flanking repeats consist only of the sequence retained after crRNA maturation. The oligos were annealed and phosphorylated using T4 polynucleotide kinase (PNK) and ligated into NcoI and HindIII sites of pHERD30T. A fragment of the resulting plasmid that includes the araC gene, pBAD promoter, and crRNA sequence was then amplified by PCR and cloned into the mini-CTX2 plasmid. The resulting constructs were then used to transform the PAO1 tn7::MbCas12a strain, and stable integration was selected for using 30 g ml⁻¹ tetracycline.

### Cloning of Candidate Anti-CRISPR Genes

All candidate genes were cloned into the pHERD30T shuttle vector, which replicates in both E. coli and P. aeruginosa. Novel genes found upstream of aca1 in Pseudomonas sp. were synthesized as gBlocks (IDT) and cloned into the SacI/PstI site of pHERD30T, which has an arabinose-inducible promoter and gentamicin selectable marker. Candidate genes derived from Moraxella bovoculi strains were amplified from the genomic DNA of 58069 and 22581 by PCR, whereas genes derived from Moraxella catarrhalis were synthesized as gBlocks (IDT). These inserts were cloned using Gibson assembly into the NcoI and HindIII sites of pHERD30T. All plasmids were sequenced using primers outside of the multiple cloning site.

### Bacteriophage Plaque Assays

Plaque assays were performed using 1.5% LB agar plates and 0.7% LB top agar, both of which were supplemented with 10 mM MgSO4. 150 ul overnight culture was resuspended in 3-4 ml molten top agar and plated on LB agar to create a bacterial lawn. Ten-fold serial dilutions of phage were then spotted onto the plate and incubated overnight at 30° C. Agar plates and/or top agar were supplemented with 0.5-1 mM isopropyl β-D-1-thiogalactopyranoside (IPTG) and 0.1-0.3% arabinose for assays performed with the LL77 (I-C) strain and with 0.1-0.3% arabinose for assays performed with the PA4386 (I-E), PA14 (I-F), and PAO1 tn7::MbCas12a (V-A) strains. Agar plates were supplemented with 50 μg ml⁻¹ gentamicin for pHERD30T retention, as specified in the text. Anti-CRISPR activity was assessed by measuring replication of the CRISPR-sensitive phages JBD30 (V-A, I-C), JBD8 (I-E) and DMS3m (I-F) on bacterial lawns relative to the vector control. JBD30, JBD8, and DMS3m are closely related phages, differing slightly at protospacer sequences. Plate images were obtained using Gel Doc EZ Gel Documentation System (BioRad) and Image Lab (BioRad) software.

### Phylogenetic Reconstructions

Homologs of AcrIF1l (accession: WP_038819808.1) were acquired through 3 iterations of psiBLASTp search the non-redundant protein database. Only hits with >70% coverage and an E value<0.0005 were included in the generation of the position specific scoring matrix (PSSM). A non-redundant set of high confidence homologs (>70% coverage, E value<0.0005) represented in unique species of bacteria were then aligned using NCBI COBALT (3) and a phylogeny was generated using the fastest minimum evolution method. The resulting phylogeny was then displayed as a phylogenetic tree using iTOL: Interactive Tree of Life (4). Similar analysis was performed to generate the phylogenetic reconstruction for AcrVA3, while BLASTp was used to generate the reconstructions for AcrVA1 and AcrVA2.

### Cloning of Constructs for Human Cell Expression

Human cell Cas12a expression plasmids were generated by sub-cloning the open-reading frames of plasmids pY014, pY117, pY010, pY016, and pY004 (Addgene plasmids 69986, 92293, 69982, 69988, and 69976, respectively; gifts from Feng Zhang) into pCAG-CFP (Addgene plasmid 11179; a gift from Connie Cepko) for wild-type MbCas12a, Mb3Cas12a, AsCas12a, LbCas12a, and FnCas12a (AAS2134, RTW2500, SQT1659, SQT1665, and AAS1472, respectively). Human cell U6 promoter expression plasmids for SpCas9 sgRNAs and Cas12a crRNAs were generated by annealing and ligating oligonucleotide duplexes into BsmBI-digested BPK1520((5), BPK3079, BPK3082 (6). BPK4446, and BPK4449 for SpCas9, AsCas12a, LbCas12a, FnCas12a, and MbCas12a/Mb3Cas12a, respectively. Human codon optimized AcrVA sequences were cloned with a c-terminal SV40 nuclear localization signal into a pCMV-T7 backbone via isothermal assembly.

### Human Cell Culture and Transfection

U2-OS cells (from Toni Cathomen, Freiburg) and U2-OS-EGFP cells (7) (containing a single integrated copy of an pCMV-EGFP-PEST reporter gene) were cultured in Advanced Dulbecco's Modified Eagle Medium supplemented with 10% heat-inactivated fetal bovine serum, 1% penicillin-streptomycin, and 2 mM GlutaMAX; a final concentration of 400 μg ml⁻¹ Geneticin was added to U2-OS-EGFP cell culture media. All cell culture reagents purchased from Thermo Fisher Scientific. Human cells were cultured at 37° C. with 5% CO₂ and were assayed bi-weekly for mycoplasma contamination. Cell line identities were confirmed by STR profiling (ATCC). All human cell electroporations were carried out using a 4-D Nucleofector (Lonza) with the SE Cell Line Kit and the DN-100 program. Unless otherwise noted, 290 ng of nuclease plasmid was co-delivered with 125 ng sgRNA/crRNA plasmid and 750 ng of anti-CRISPR protein plasmid. Conditions listed as “filler DNA” include 750 ng of an incompatible nuclease expression plasmid (SpCas9 for Cas12a experiments, or AsCas12a for SpCas9 experiments) to ensure electroporation of consistent DNA quantities. Control conditions for both EGFP disruption and endogenous targeting included nuclease expression plasmids co-delivered with a U6-null plasmid (in place of sgRNA/crRNA plasmids). For AcrIIA4 titration experiments with SpCas9, a pCAG-SpCas9 plasmid was used (SQT817) (8) for a comparable vector architecture relative to Cas12a expression plasmids.

### Human Cell Nuclease Assays

EGFP disruption experiments were performed essentially as previously described (7). Briefly, cells were electroporated as described above and were analyzed ˜52 h post-nucleofection for EGFP levels using a Fortessa flow cytometer (BD Biosciences). Background EGFP loss in negative control conditions was approximately 3% (represented as a red dashed line in figures). For T7 endonuclease I (T7E1) assays, human U2-OS cells were electroporated as described above and genomic DNA (gDNA) was extracted approximately 72 hours post-nucleofection using a custom lysis and paramagnetic bead extraction. Paramagnetic beads were prepared similar to as previously described (9): GE Healthcare Sera-Mag SpeedBeads (Thermo Fisher Scientific) were washed in 0.1×TE and suspended in 20% PEG-8000 (w/v), 1.5 M NaCl, 10 mM Tris-HCl pH 8, 1 mM EDTA pH 8, and 0.05% Tween20. To lyse cells, cells were washed with PBS and then subsequently incubated at 55° C. for 12-20 hours in 200 μL lysis buffer (100 mM Tris HCl pH 8.0, 200 mM NaCl, 5 mM EDTA, 0.05% SDS, 1.4 mg/mL Proteinase K (New England Biolabs, NEB), and 12.5 mM DTT). The cell lysate was mixed with 165 μL paramagnetic beads and then separated on a magnetic plate. Beads were washed with 70% three times and were permitted to dry on a magnetic plate for 5 minutes before elution with 65 μL elution buffer (1.2 mM Tris-HCl pH 8.0). To perform T7E1 assays, genomic loci were amplified by PCR using ˜100 ng of genomic DNA (gDNA), Hot Start Phusion Hex DNA Polymerase (NEB). PCR products were visualized on a QIAxcel capillary electrophoresis instrument (Qiagen) to confirm amplicon size and purity, and were subsequently purified using paramagnetic beads. T7E1 assays were performed as previously described (7) to approximate nuclease modification of targeted genomic loci. Briefly, 200 ng purified PCR product was denatured, annealed, and digested with 10U T7E1 (NEB) at 37° C. for 25 minutes. Digested amplicons were purified with paramagnetic beads and quantified using a QIAxcel capillary electrophoresis machine (Qiagen) to estimate target site modification.

### REFERENCES CITED IN MATERIALS AND METHODS

1. J. Söding, A. Biegert, A. N. Lupas, The HHpred interactive server for protein homology detection and structure prediction. Nucleic Acids Res. 33, W244-8 (2005).
2. B. Zetsche et al., Cpf1 is a single RNA-guided endonuclease of a class 2 CRISPR-Cas system. Cell. 163, 759-771 (2015).
3. J. S. Papadopoulos, R. Agarwala, COBALT: constraint-based alignment tool for multiple protein sequences. Bioinformatics. 23, 1073-1079 (2007).
4. I. Letunic, P. Bork, 20 years of the SMART protein domain annotation resource. Nucleic Acids Res. 46, D493-D496 (2018).
5. B. P. Kleinstiver et al., Engineered CRISPR-Cas9 nucleases with altered PAM specificities. Nature. 523, 481-485 (2015).
6. B. P. Kleinstiver et al., Genome-wide specificities of CRISPR-Cas Cpf1 nucleases in human cells. Nat. Biotechnol. 34, 869-874 (2016).
7. D. Reyon et al., FLASH assembly of TALENs for high-throughput genome editing. Nat. Biotechnol. 30, 460-465 (2012).
8. S. Q. Tsai et al., Dimeric CRISPR RNA-guided FokI nucleases for highly specific genome editing. Nat. Biotechnol. 32, 569-576 (2014).
9. N. Rohland, D. Reich, Cost-effective, high-throughput DNA sequencing libraries for multiplexed target capture. Genome Res. 22, 939-946 (2012).

### Example 2

### Discovery:

A bioinformatics pipeline was prepared that searched for self-targeting in prokaryotic genomes. A “self-target” is the co-occurrence of a nucleotide sequence both as a spacer in a CRISPR array and somewhere else in the genome outside of any CRISPR array. These “self-targeting” spacers should allow the natural CRISPR systems to self-target the genome, which is typically lethal. The hypothesis is that these “self-targets” can only exist in genomes where anti-CRISPRs exist. Thus, the bioinformatic pipeline identifies a list of genomes potentially containing anti-CRISPRs for various CRISPR systems (based on the array/source of the self-target).

The bioinformatics pipeline identified a number of genomes that had self-targeting. We focused on Cas12a (Cpf1), as it is a major genome editing tool and no anti-CRISPRs had been discovered for it. Looking specifically at Cas12a, roughly 20 genomes with self-targeting were identified, including a set of Moraxella bovoculi genomes that were highly promising.

### Screening

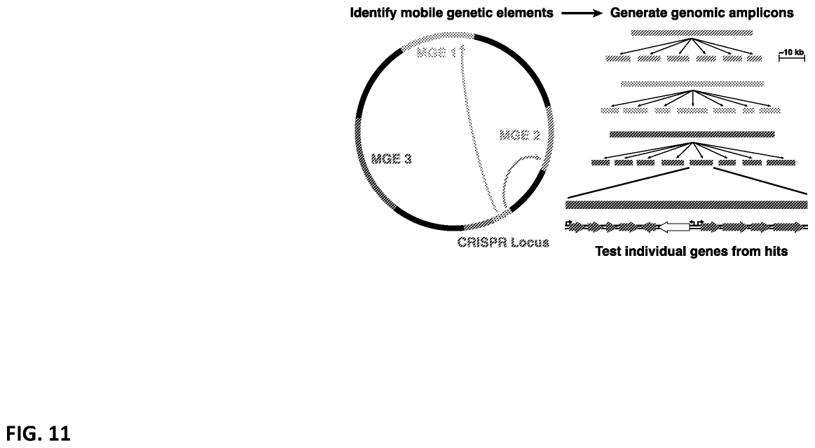

FIG. 11 shows a strategy to produce genomic fragments to test for anti-CRISPRs in self-targeting M. bovoculi genomes. To locate anti-CRISPRs in the self-targeting M. bovoculi genomes, bioinformatic tools were used to predict the mobile genetic elements (MGEs; plasmids, prophages, transposons, etc.) in each of the self-targeting genomes with self-targeting from a Cas12a array (strains 33362, 58069, 58086). These MGEs were predicted first because all of the known anti-CRISPRs at the time had been found in these regions. PCR was then used to amplify the predicted MGEs in ˜10 kb fragments to test each fragment for anti-CRISPR activity.

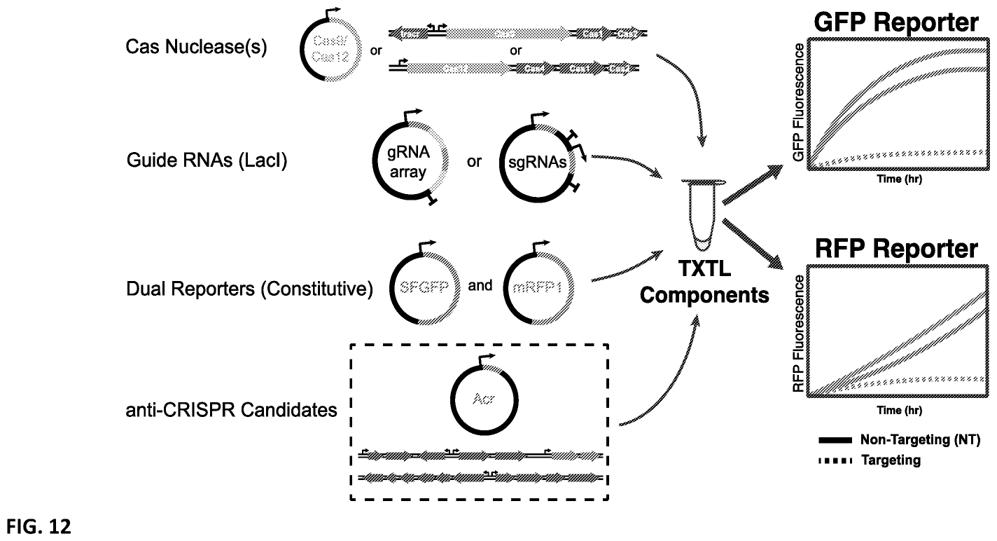

To test each fragment, a cell-free reaction system was set up using a transcription-translation (TXTL) system (based on E. coli S30 extracts) where two fluorescent reporters (GFP and RFP) are co-expressed with Cas12a and guide RNAs targeting both reporters (all from DNA) (FIG. 12, below). Without anti-CRISPR activity, the Cas12a and gRNAs are expressed and target the reporter plasmids, cleaving them and preventing reporter expression. With anti-CRISPR activity, the Cas12a would be inhibited, and the reporters are expressed, producing a fluorescence curve over time as the reaction proceeds.

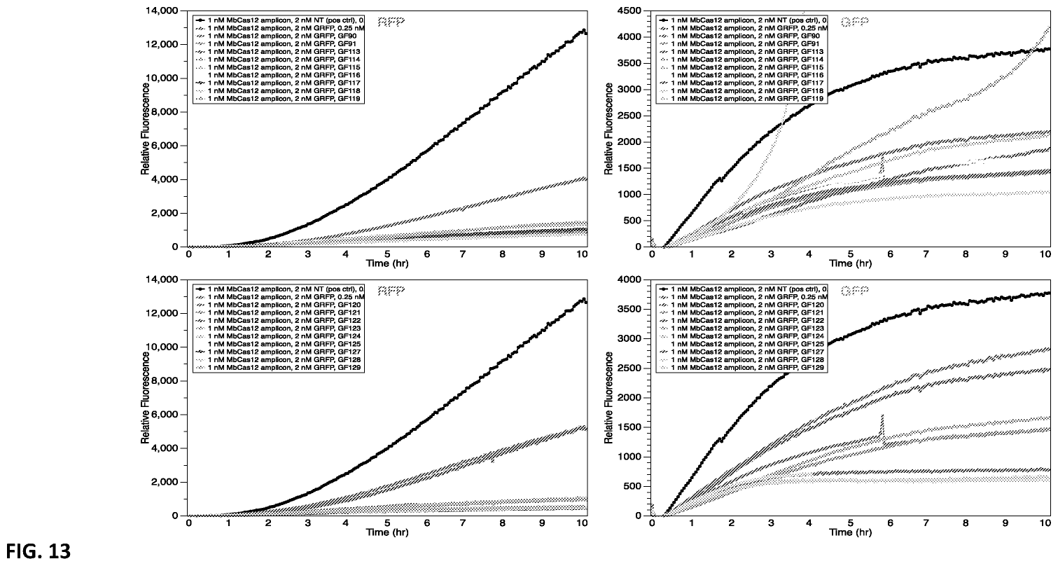

After testing the genomic fragments from M. bovoculi, four fragments were identified that exhibited anti-CRISPR activity, with three of them being unique (see, SEQ ID NOS: 2, 3, and 4; FIG. 13).

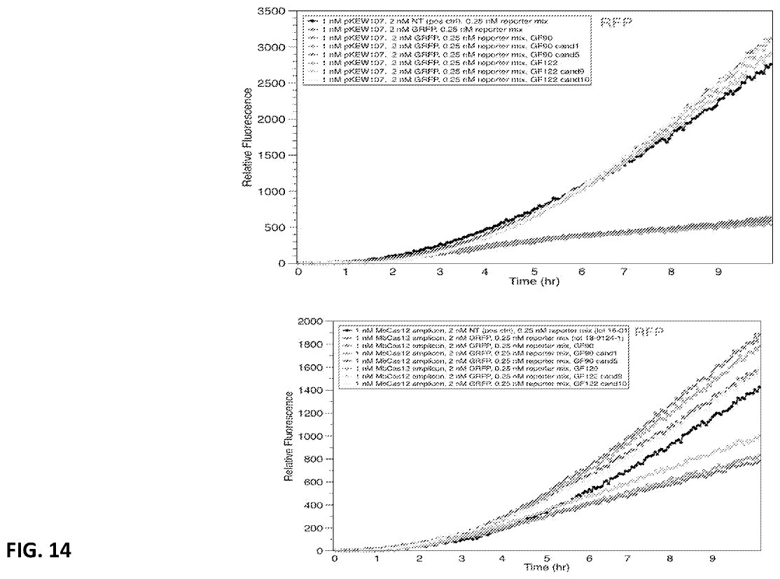

For each of these fragments, subfragments were amplified and tested to arrive at shorter stretches of DNA containing the activity. At this point, the individual genes were cloned into an expression vector and tested each gene with the TXTL system. Three unique genes were ultimately identified that inhibited Cas12a activity in the TXTL system (FIG. 14).

### Confirmation

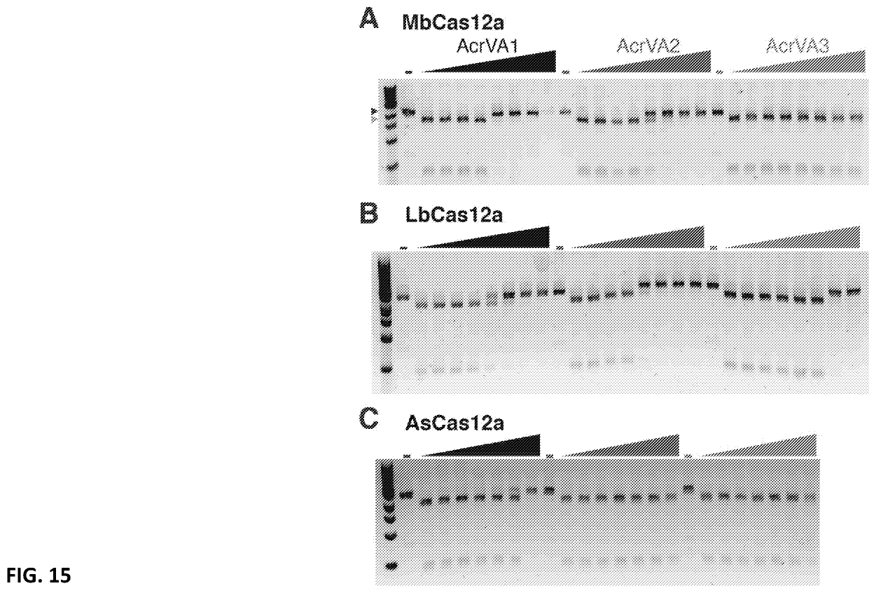

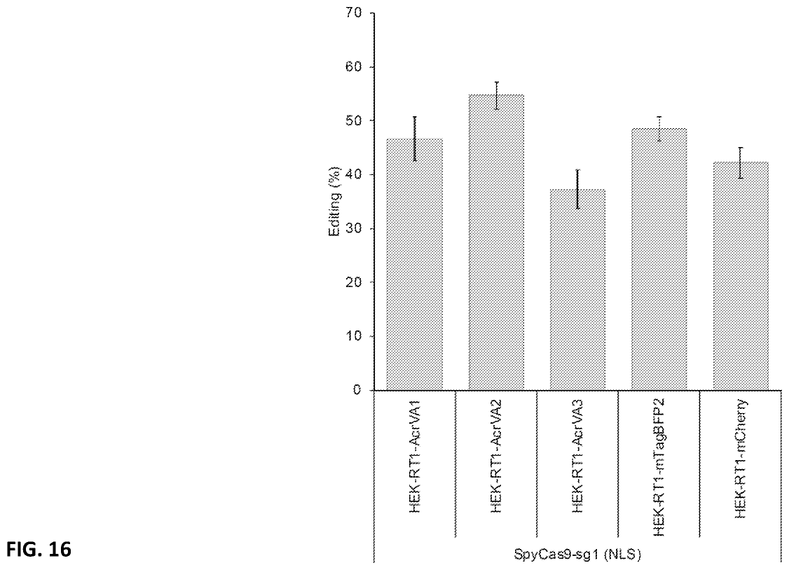

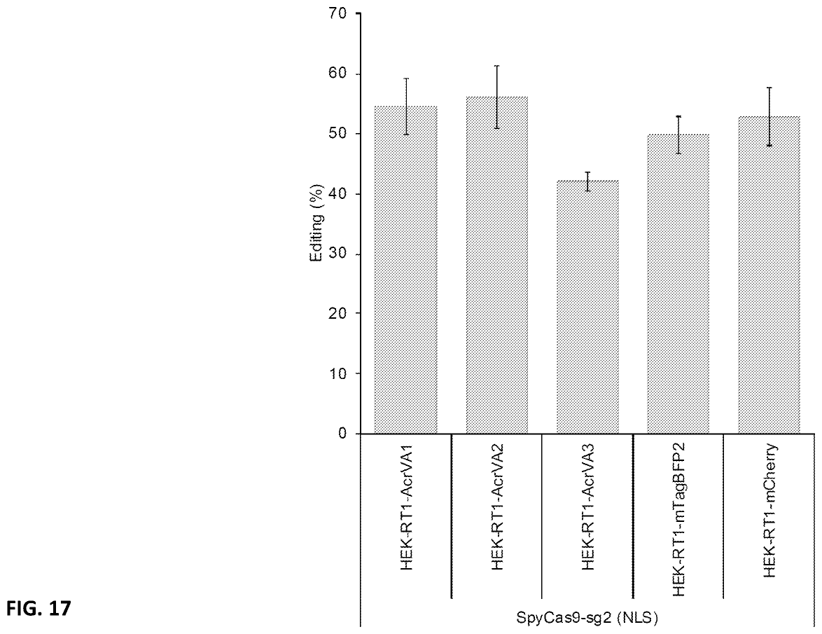

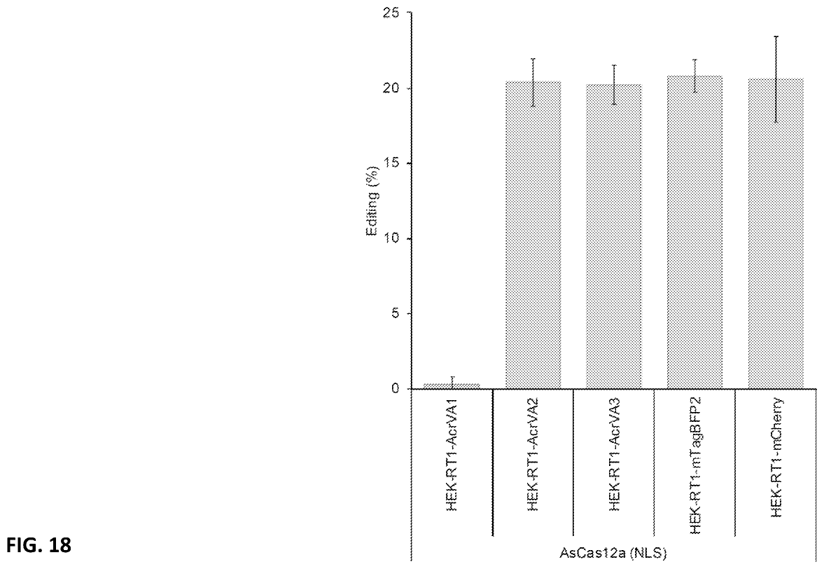

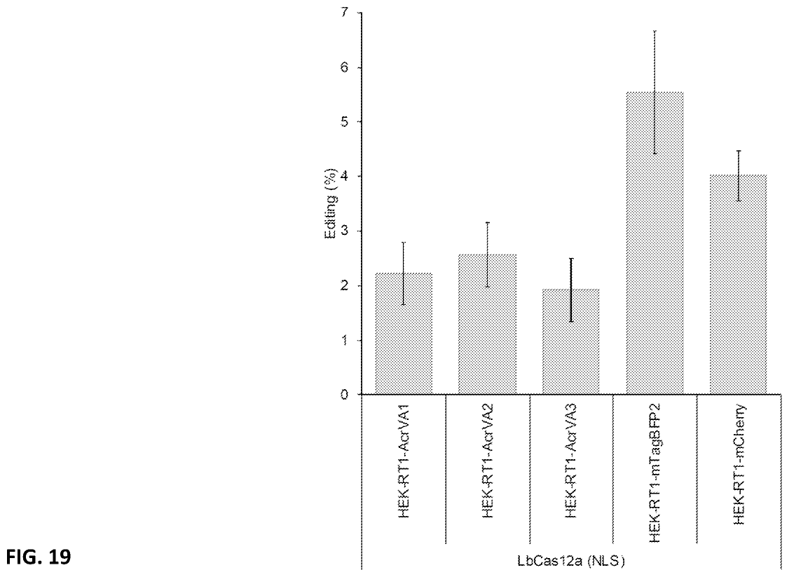

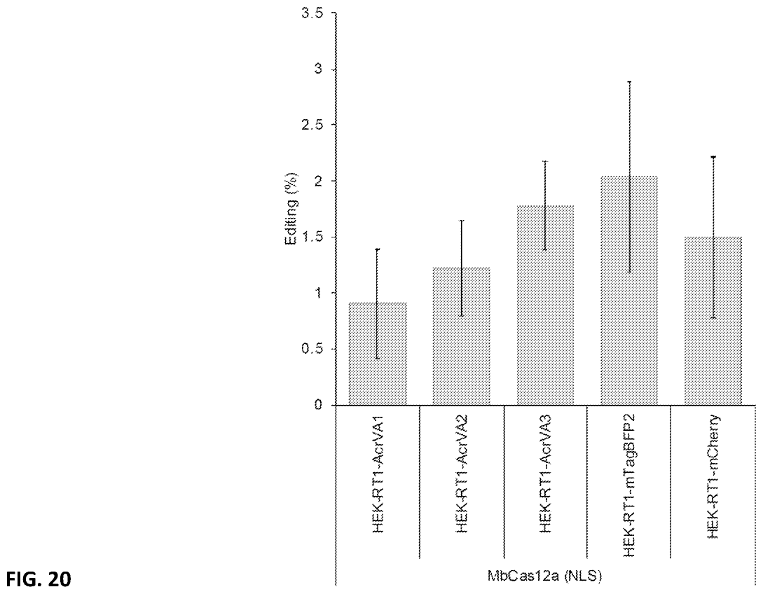

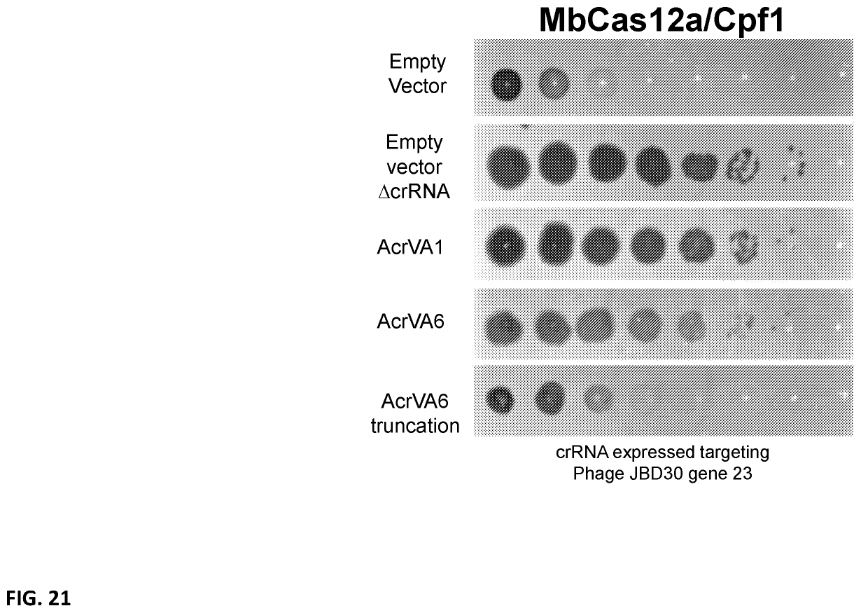

After identifying these three proteins by TXTL screening, each protein was purified and a set of in vitro cleavage inhibition assays were performed to confirm the anti-CRISPR activity. Each of the three anti-CRISPR candidate proteins were tested against three different Cas12as: from M. bovoculi (anti-CRISPR source organism), Lachnospiraceae bacterium (commonly used in gene editing), and Acidaminococcus sp. BV3L6 (commonly used in gene editing) (FIG. 15).

In the cleavage experiment, 5 nM (final) of linearized plasmid was mixed with varying concentrations of anti-CRISPR candidate from 0 nM to 1.25 μM in 1× cleavage buffer and incubated at 37° C. for 10 min. RNP was then added to start the cleavage reaction (25 nM of RNP final), which was incubated at 37° C. for 30 min. The reaction was then quenched and run on a 1% agarose gel to produce the image in FIG. 15. All three proteins each inhibited at least one of the Cas12a proteins, confirming they are all anti-CRISPR genes.

### Inhibition in Human Cell Editing

SpyCas9 was an editing control and we observed excellent inhibition of AsCas12 with AcrVA1 (gene 1) and moderate (incomplete) inhibition of LbCas12 with three Acrs (SEQ ID NOS: 2, 3, and 4). Five human cell lines (HEK293T) were stably expressing one of the following: AcrVA1, AcrVA2, AcrVA3, BFP, or mCherry (see FIGS. 10-14, right to left on each chart's x-axis). Each separate plot represents a different RNP that was delivered targeting an inducible eGFP gene in the genome.

There are two plots where SpyCas9 was delivered and all of the bars are high, indicating that we were able to edit all five strains and none of the AcrVA genes or the BFP/RFP controls inhibited editing. There are also plots for MbCas12, LbCas12, and AsCas12, where the latter two are the most commonly used Cas12s in biotech applications. We saw weak editing in MbCas12 (which follows the observations from the original Cas12/Cpf1 discovery paper Zetsche, 2015), moderate editing in LbCas12, where all three AcrVA genes exhibited ˜50% inhibition of editing, and good editing with AsCas12, where AcrVA1 was very effective and AcrVA2/3 did not inhibit at all.

### Materials and Methods

Bioinformatics with Self-Targeting Spacer Searcher (STSS)

The Self-Target Spacer Searcher is a cross-platform python script (available at github.com/kew222/Self-Targeting-Spacer-Search-tool/releases for public use) that accepts a search query for the NCBI Genome database and returns a list of self-targeting spacers found within the genomes found from the query. Many of the parameters specifically described below can be adjusted at runtime.

The search term ‘Prokaryote’ was provided to search NCBI's Genome database, which was linked to nucleotide through assembly to download all of the resulting genomes in fasta format. CRISPR arrays were then predicted for each genome using the CRISPR Recognition Tool (CRT) using 18 and 45 as minimum and maximum repeat and spacer lengths, respectively, and a minimum repeat length of four. For each array that was predicted, the spacers were collected and used to BLAST (blastn with default settings) all of the contigs within the array's assembly. Any hit to a contig in the assembly was considered a self-target, except for the DNA bases within all of the predicted arrays, plus an additional 500 bp from each end of the predicted array, which were ignored. Long stretches of degenerate bases were also artificially shrunk to under 500 bp, as CRT is unable to process these sequences.

For each self-targeting spacer that was found, a set of data was collected about the source locus and the genomic self-target position. To collect these data, the Genbank file for each self-targeting genome was downloaded and all of the genes within 20 kb of the spacer within the array were compared to Hidden Markov Models (HMMs) for many of the known Cas proteins using HMMER v3 with an e-value cutoff of 10⁻⁶ to call Cas proteins near the array. The list of Cas proteins was then used to try to predict the CRISPR subtype of the array based on the composition of the nearby Cas proteins, using previously coined definitions (see, e.g., Makarova (2011) and (2015) for review). The CRISPR subtype was predicted by enumerating the number of possible types each identified Cas protein could belong to and choosing the subtype with the great number of hits. The exact definitions chosen can be found in CRISPR_definitions.py within STSS. Similarly, the Cas protein HMMs are also found within STSS.

After searching for Cas proteins, the repeats and spacers from CRISPR array were also examined. First, all spacers in the self-targeting array were aligned with Clustal Omega to check for conserved bases at each end of the spacer, to check for the possibility that the array predicted by CRT miscalled the repeat sequence. If the array contained at least six repeats and a string of bases at either end contained 75% or more of the same base, those bases were assumed to be part of the repeat sequence and both the repeat and spacer sequences were adjusted appropriately. Arrays with four or five repeats used 100% as the cutoff to correct the repeat sequence. Additionally, if the length of the longest and shortest spacers within an array differed by more than 25%, the array was rejected as non-CRISPR, as they possibly represented a direct repeat sequence or other DNA feature. If passing the length variance filter, the consensus repeat sequence was determined using Biopython's dumb_consensus( ) method and any mutations/indels in the repeat sequences flanking the self-targeting spacer were reported.

To predict the subtype of CRISPR system the array of a self-targeting spacer belonged to (in addition to the protein method described above), the self-targeting spacer was compared to a set of HMMs that were built from the REPEATS dataset from CRISPRmap and additional multiple-sequence alignments for more recently discovered CRISPR systems, such as the type V and type VI systems. These HMMs are also available in STSS.

The orientation of the array was determined first using the direction provided in repeat sequence HMMs if the consensus sequence produced a hit. Otherwise, the CRISPR array was assumed to be oriented such that it was downstream of the predicted Cas proteins, but only if a single subtype was predicted. If neither of these conditions were met, the array direction was left in the default orientation given by CRT (i.e. forward, on the top strand).

To analyze the genomic target of the self-targeting spacer, we took the spacer sequence (possibly corrected from the array analysis) and performed a gapless BLAST at the target site to force the comparison of mutations only and exclude indels in the alignment, as we would not expect bulging to occur in the Cas proteins. The gapless BLAST positions were used as the final alignment and nine bases up- and downstream of the target were reported as potential PAM sequences. Because of the possibility that the predicted CRISPR subtypes in earlier stages are incorrect (or there are multiple), and because there are myriad systems for which no PAM has been experimentally validated (especially in type II), no assumptions about what the expected PAM was were made, nor which side of the protospacer it should occur on. At this stage, we performed a second heuristic filtering step to remove potential falsely predicted CRISPR arrays by checking the sequences up- and downstream of the protospacer and comparing them to the consensus repeat. If eight of the nine bases matched on either side of the protospacer, the potential self-target was rejected as being in a missed array or part of a direct repeat sequence, etc. that escaped the length variance filter.

The last part of STSS analysis was to check the contig the targeted DNA occurred in for the presence of MGEs. As part of the STSS pipeline, we searched for prophages in the contig using the online webserver provided by PHASTER and noted if there were prophages present and what which prophage the self-target occurred in if so. PHASTER analysis completed the STSS pipeline; however, we also used the Islander Database to locate predicted MGEs near the self-target sequence. Regardless of whether an MGE was predicted or not, the feature (or features if the protospacer fell between genes) targeted by the self-targeting spacer was reported. If that gene was labeled as ‘hypothetical protein’, it was analyzed for potential conserved sequence on NCBI's CD-Search webserver. All of the data collected in the steps described above was output in a text format.

After the STSS data was collected, we performed a manual scan of the results to correct any potentially miscalled repeat/spacer sequences. Additionally, we examined the unknown type II self-targeting spacers. With the methods used above, we were unable to call type II-C separately from II-A or II-B. To correct this, we manually annotated the type II-C systems based on homology of the Cas9 to other known II-C Cas9s as well as the repeat sequence. Because the type II-C array is in the inverse orientation relative to most CRISPR arrays, we also needed to manually adjust that orientation, which is noted in Data S1 with green highlighting and a note in the orientation column.

To determine which genomes contained an Acr gene, a compiled list of the known Acr genes was used to BLAST against all NCBI genomes with an E-value limit of 104. All genes passing this cutoff were annotated as anti-CRISPRs.

### Analysis of Self-Targeting and Anti-CRISPR Co-Occurrence

Self-targeting spacers derived from the type I-E and type I-F CRISPR system of Pseudomonas aeruginosa, type 11-A system of Listeria monocytogenes, and type II-C system of Neisseria meningitidis were selected from the full STSS dataset to determine the level of co-occurrence. Self-targeting spacers were included as long as there was reasonable evidence that it belonged to one of the above four systems, using the identified Cas proteins and repeat sequences (via HMM or by inspection). Spacers whose target occurred on the edge of the contig such that no PAM sequences were available were excluded. Genomes without protein annotations were also ignored.

In order for a self-targeting spacer to be expected to be lethal it was required to meet three conditions: 1) all Cas surveillance proteins needs to be present (and not marked as a pseudogene), 2) no more than two mismatches in the target sequence, and 3) the target must have the correct PAM sequence. The PAM requirements differed for each system. The L. monocytogenes system was required to have a perfect NRG PAM and the P. aeruginosa systems required perfect PAMs of AAG or CC for the type I-E and I-F systems, respectively. Due to the longer requirement, we allowed the NNNNGATT PAM for the type II-C system to contain one mismatch or indel.

Using the list of spacers, lists of genomes for each CRISPR system were compiled where each genome contained: at least one self-targeting spacer, at least one lethal self-targeting spacer, and at least one lethal self-targeting spacer and anti-CRISPR.

### Selecting Genomes to Search for Cas12 Anti-CRISPRs

Within the results from STSS, we searched for type V-A self-targets that contained Cas12 near the array, no mismatches between the spacer and target sequences, and preferentially occurred within a predicted MGE. While a few type V self-targeting genomes were apparent, we observed a group of genomes with unique spacer sequences from Moraxella bovoculi that met the ideal conditions, especially strain 22581, which contained multiple self-targeting spacers from the type V-A array in the genome.

### Genomic DNA Extraction

To extract gDNA, 4 mL of M. bovoculi cells (strains 22581, 33362, and 58069) were grown overnight in BHI media supplemented with 30 mM NaCl and pelleted. The pellets were resuspended in 300 μL of TE buffer, transferred to a 2 mL bead beating tube where 100 mg of 0.1 mm glass beads were added before beating for 90 seconds three times with 30 seconds on ice between each beating. The lysate was then used to purify the genomic DNA using the EZNA (Omega), following the manufacturer's instructions.

### DNA Preparation for TXTL

The TXTL reactions contained up to four DNA components: the reporter plasmids (for GFP and RFP), a Cas12 genomic amplicon, a gRNA plasmid, and an optional anti-CRISPR candidate amplicon or plasmid. The two reporter plasmids were minimal plasmids containing an Amp resistance gene, ColE1 origin, and a consensus E. coli σ⁷⁰ promoter preceding either mRFP1 or superfolder GFP (SFGFP). The gRNA plasmids were built from the same vector as the reporter plasmids, except that the fluorescent reporters were replaced with LacI and a synthetic array following a P_{Lac} promoter containing either: three repeats interspersed with spacers targeting GFP and RFP or two repeats with a non-targeting (NT) spacer. For Cas12 expression, we prepared a genomic amplicon from M. bovoculi strain 22581 that contained Cas12, Cas1, Cas2, and Cas4, stopping short of the genomic array sequence. Genomic amplicons or subfragments were generated using PCR (described below). Individual Acr candidate genes were cloned into the same vector as the reporter plasmids, replacing the reporter with TetR and a P_{Tet} promoter followed by the candidate protein with its genomic ribosome binding site and a strong terminator. See Table 6 for plasmid sequences.

TABLE 6

| DNA/RNA | Sequence |
|---|---|
| Chi6 DNA | TCACTTCACTGCTGGTGGCCACTGCTGGTGGCCACTGCTGGTGGCCACTGCTGGTGGCCACTGCTGGTGGCCACTGCTGGT |
| (forward) | GGCCA |
| Chi6 DNA | TGGCCACCAGCAGTGGCCACCAGCAGTGGCCACCAGCAGTGGCCACCAGCAGTGGCCACCAGCAGTGGCCACCAGCAGTGA |
| (reverse) | AGTGA |
| SFGFP | atgagcaaaggagaagaacttttcactggagttgtcccaattcttgttgaattagatggtgatgttaatgggcacaaattt |
| sequence | tctgtccgtggagagggtgaaggtgatgctacaaacggaaaactcacccttaaatttatttgcactactggaaaactacct |
| | gttccgtggccaacacttgtcactactctgacctatggtgttcaatgcttttcccgttatccggatcacatgaaacggcat |
| | gactttttcaagagtgccatgcccgaaggttatgtacaggaacgcactatatctttcaaagatgacgggacctacaagacg |
| | cgtgctgaagtcaagtttgaaggtgatacccttgttaatcgtatcgagttaaagggtattgattttaaagaagatggaaac |
| | attcttggacacaaactcgagtacaactttaactcacacaatgtatacatcacggcagacaaacaaaagaatggaatcaaa |
| | gctaacttcaaaattcgccacaacgttgaagatggttccgttcaactagcagaccattatcaacaaaatactccaattggc |
| | gatggccctgtccttttaccagacaaccattacctgtcgacacaatctgtcctttcgaaagatcccaacgaaaagcgtgac |
| | cacatggtccttcttgagtttgtaactgctgctgggattacacatggcatggatgagctctacaaa |
| mRFP1 | atggcgagtagcgaagacgttatcaaagagttcatgcgtttcaaagttcgtatggaaggttccgttaacggtcacgagttc |
| sequence | gaaatcgaaggtgaaggtgaaggtcgtccgtacgaaggtactcagaccgctaaactgaaagttaccaaaggtggtccgctg |
| | ccgttcgcttgggacatcctgtccccgcagttccagtacggttccaaagcttacgttaaacacccggctgacatcccggac |
| | tacctgaaactgtccttcccggaaggtttcaaatgggaacgtgttatgaacttcgaagacggtggtgttgttaccgttacc |
| | caggactcctccctgcaagacggtgagttcatctacaaagttaaactgcgtggtactaacttcccgtccgacggtccggtt |
| | atgcagaaaaaaaccatgggttgggaagcttccaccgaacgtatgtacccggaagacggtgctctgaaaggtgaaatcaaa |
| | atgcgtctgaaactgaaagacggtggtcactacgacgctgaagttaaaaccacctacatggctaaaaaaccggttcagctg |
| | ccgggtgcttacaaaaccgacatcaaactggacatcacctcccacaacgaagactacaccatcgttgaacagtacgaacgt |
| | gctgaaggtcgtcactccaccggtgcttaa |
| MbCas12 | gtctaacgaccttttaaatttctactgtttgtagat |
| repeat | |
| GFP spacer | CACTGGAGTTGTCCCAATTCTTGT |
| sequence | |
| RFP spacer | AAAGTTCGTATGGAAGGITCCGTT |
| sequence | |
| MbCas12 | taatacgactcactataggctaacgaccttttaaatttctactgtttg |
| IVT template | |
| primer 1 | |
| MbCas12 | tttccaatgatgagcactttatctacaaacagtagaaatttaaaaggtcg |
| IVT template | |
| primer 2 | |
| LbCas12 IVT | taatacgactcactataggtttcaaagattaaataatttctactaagtg |
| template | |
| primer 1 | |
| LbCas12 IVT | tttccaatgatgagcactttatctacacttagtagaaattatttaatctttgaaac |
| template | |
| primer 2 | |
| AsCas12 IVT | taatacgactcactataggtcaaaagacctttttaatttctactc |
| template | |
| primer 1 | |
| AsbCas12 | tttccaatgatgagcactttatctacaagagtagaaattaaaaaggtcttttgac |
| IVT template | |
| primer 2 | |
| Cas12 gRNA | ctcccttagccatccgagtggacgacgtcctccttcggatgcccaggtcggaccgcgaggaggtggagatgccatgccgac |
| template | cctttccaatgatgagcac |
| reverse | |
| primer | |
| MbCas12 | ggctaacgaccttttaaatttctactgtttgtagataaagtgctatcattggaaa |
| AmpR gRNA | |
| LbCas12 | ggtttcaaagattaaataatttctactaagtgtagataaagtgctatcattggaaa |
| AmpR gRNA | |
| AsCas12 | ggtcaaaagacctttttaatttctatcttgtagataaagtgctatcattggaaa |
| AmpR gRNA | |
| Linear DNA | aattctaaagatctttgacagctagctcagtcctaggtataatactagtgcctctacctgcttcggccgataaagccgacg |
| target | ataatactcccaaagcccgccgaaaggcgggcttttttttggatccttactcgagtctagactgcaggcttcctcgctcac |
| | tgactcgctgcgctcggtcgttcggctgcggcgagcggtatcagctcactcaaaggcggtaatacggttatccacagaatc |
| | aggggataacgcaggaaagaacatgtgagcaaaaggccagcaaaaggccaggaaccgtaaaaaggccgcgttgctggcgtt |
| | tttccacaggctccgcccccctgacgagcatcacaaaaatcgacgctcaagtcagaggtggcgaaacccgacaggactata |
| | aagataccaggcgtttccccctggaagctccctcgtgcgctctcctgttccgaccctgccgcttaccggatacctgtccgc |
| | ctttctcccttcgggaagcgtggcgctttctcatagctcacgctgtaggtatctcagttcggtgtaggtcgttcgctccaa |
| | gctgggctgtgtgcacgaaccccccgttcagcccgaccgctgcgccttatccggtaactatcgtcttgagtccaacccggt |
| | aagacacgacttatcgccactggcagcagccactggtaacaggattagcagagcgaggtatgtaggcggtgctacagagtt |
| | cttgaagtggtggcctaactacggctacactagaagaacagtatttggtatctgcgctctgctgaagccagttaccttcgg |
| | aaaaagagttggtagctcttgatccggcaaacaaaccaccgctggtagcggtggtttttttgtttgcaagcagcagattac |
| | gcgcagaaaaaaaggatctcaagaagatcctttgatcttttctacggggtctgacgctcagtggaacgaaaactcacgtta |
| | agggattttggtcatgagattatcaaaaaggatcttcacctagatccttttaaattaaaaatgaagttttaaatcaatcta |
| | aagtatatatgagtaaacttggtctgacagttaccaatgcttaatcagtgaggcacctatctcagcgatctgtctatttcg |
| | ttcatccatagttgcctgactccccgtcgtgtagataactacgatacgggagggcttaccatctggccccagtgctgcaat |
| | gataccgcgagacccacgctcaccggctccagatttatcagcaataaaccagccagccggaagggccgagcgcagaagtgg |
| | tcctgcaactttatccgcctccatccagtctattaattgttgccgggaagctagagtaagtagttcgccagttaatagttt |
| | gcgcaacgttgttgccattgctacaggcatcgtggtgtcacgctcgtcgtttggtatggcttcattcagctccggttccca |
| | acgatcaaggcgagttacatgatcccccatgttgtgcaaaaaagcggttagctccttcggtcctccgatcgttgtcagaag |
| | taagttggccgcagtgttatcactcatggttatggcagcactgcataattctcttactgtcatgccatccgtaagatgctt |
| | ttctgtgactggtgagtactcaaccaagtcattctgagaatagtgtatgcggcgaccgagttgctcttgcccggcgtcaat |
| | acgggataataccgcgccacatagcagaactttaaaagtgctcatcattggaaaacgttcttcggggcgaaaactctcaag |
| | gatcttaccgctgttgagatccagttcgatgtaacccactcgtgcacccaactgatcttcagcatcttttactttcaccag |
| | cgtttctgggtgagcaaaaacaggaaggcaaaatgccgcaaaaaagggaataagggcgacacggaaatgttgaatactcat |
| | actcttcctttttcaatattattgaagcatttatcagggttattgtctcatgagcggatacatatttgaatgtatttagaa |
| | aaataaacaaataggggttccgcgcacatttccccgaaaagtgccacctgacgtctaagaaaccattattatcatgacatt |
| | aacctataaaaataggcgtatcacgaggcagaatttcagataaaaaaaatccttagctttcgctaaggatgatttctgg |
| pTet Acr | gaattcaactgcaggcactgcccatggacctcggtaccgaatagctagccggtaatgcattcgctagagctcctaaagcat |
| gene | gcgacctgcaaccggtctgtcacgtacgtcgccaccgtcgacgtcgttcgtaagtagcctagataaataaaataatcagtt |
| expression | aaccgcgagccccatgcgagagtagggaactgccaggcatttcagccaaaaaacttaagaccgccggtcttgtccactacc |
| plasmid | ttgcagtaatgcggtggacaggatcggcggttttcttttctcttctcaaccgccgggagcggatttgaacgttgcgaagca |
| (genes | acggcccggagggtggcgggcaggacgcccgccataaactgccaggcatcaaattaagcagaaggccatcctgacggatgg |
| inserted at | cctttttgcgtttctacaaactctgcggtaatacggttatccacagaatcaggggataacgcaggaaagaacatgtgagca |
| XXXX) | aaaggccagcaaaaggccaggaaccgtaaaaaggccgcgttgctggcgtttttccacaggctccgcccccctgacgagcat |
| | cacaaaaatcgacgctcaagtcagaggtggcgaaacccgacaggactataaagataccaggcgtttccccctggaagctcc |
| | ctcgtgcgctctcctgttccgaccctgccgcttaccggatacctgtccgcctttctcccttcgggaagcgtggcgctttct |
| | catagctcacgctgtaggtatctcagttcggtgtaggtcgttcgctccaagctgggctgtgtgcacgaaccccccgttcag |
| | cccgaccgctgcgccttatccggtaactatcgtcttgagtccaacccggtaagacacgacttatcgccactggcagcagcc |
| | actggtaacaggattagcagagcgaggtatgtaggcggtgctacagagttcttgaagtggtggcctaactacggctacact |
| | agaaggacagtatttggtatctgcgctctgctgaagccagttaccttcggaaaaagagttggtagctcttgatccggcaaa |
| | caaaccaccgctggtagcggtggtttttttgtttgcaagcagcagattacgcgcagaaaaaaaggatctcaagaagatcct |
| | ttgatcttttctacggggtctgacgctcagtggaacgaaaactcacgttaagggattttggtcatgagattatcaaaaagg |
| | atcttcacctagatccttttaaattaaaaatgaagttttaaatcaatctaaagtatatatgagtaaacttggtctgacagt |
| | taccaatgcttaatcagtgaggcacctatctcagcgatctgtctatttcgttcatccatagttgcctgactccccgtcgtg |
| | tagataactacgatacgggagggcttaccatctggccccagtgctgcaatgataccgcgagacccacgctcaccggctcca |
| | gatttatcagcaataaaccagccagccggaagggccgagcgcagaagtggtcctgcaactttatccgcctccatccagtct |
| | attaattgttgccgggaagctagagtaagtagttcgccagttaatagtttgcgcaacgttgttgccattgctacaggcatc |
| | gtggtgtcacgctcgtcgtttggtatggcttcattcagctccggttcccaacgatcaaggcgagttacatgatcccccatg |
| | ttgtgcaaaaaagcggttagctccttcggtcctccgatcgttgtcagaagtaagttggccgcagtgttatcactcatggtt |
| | atggcagcactgcataattctcttactgtcatgccatccgtaagatgcttttctgtgactggtgagtactcaaccaagtca |
| | ttctgagaatagtgtatgcggcgaccgagttgctcttgcccggcgtcaatacgggataataccgcgccacatagcagaact |
| | ttaaaagtgctcatcattggaaaacgttcttcggggcgaaaactctcaaggatcttaccgctgttgagatccagttcgatg |
| | taacccactcgtgcacccaactgatcttcagcatcttttactttcaccagcgtttctgggtgagcaaaaacaggaaggcaa |
| | aatgccgcaaaaaagggaataagggcgacacggaaatgttgaatactcatactcttcctttttcaatattattgaagcatt |
| | tatcagggttattgtctcatgagcggatacatatttgaatgtatttagaaaaataaacaaataggggttccgcgcacattt |
| | ccccgaaaagtgccacctgacgtctaagaaaccattattgtggatataccttactcgagttagccttgataggacgtctta |
| | agacccactttcacatttaagttgtttttctaatccgcatatgatcaattcaaggccgaataagaaggctggctctgcacc |
| | ttggtgatcaaataattcgatagcttgtcgtaataatggcggcatactatcagtagtaggtgtttccctttcttctttagc |
| | gacttgatgctcttgatcttccaatacgcaacctaaagtaaaatgccccacagcgctgagtgcatataatgcattctctag |
| | tgaaaaaccttgttggcataaaaaggctaattgattttcgagagtttcatactgtttttctgtaggccgtgtacctaaatg |
| | tacttttgctccatcgcgatgacttagtaaagcacatctaaaacttttagcgttattacgtaaaaaatcttgccagctttc |
| | cccttctaaagggcaaaagtgagtatggtgcctatctaacatctcaatggctaaggcgtcgagcaaagcccgcttattttt |
| | tacatgccaatacaatgtaggctgctctacacctagcttctgggcgagtttacgggttgttaaaccttcgattccgacctc |
| | attaagcagctctaatgcgctgttaatcactttacttttatctaatctagacatcattaattcctaatttttgttgacact |
| | ctatcgttgatagagttattttaccactccctatcagtgatagagaaaaXXXX |
| Cas12 non- | gaattctaaagatctggcacgtaagaggttccaactttcaccataatgaaacatactagagaaagaggagaaatactagat |
| targeting | ggtgaatgtgaaaccagtaacgttatacgatgtcgcagagtatgccggtgtctcttatcagaccgtttcccgcgtggtgaa |
| gRNA | ccaggccagccacgtttctgcgaaaacgcgggaaaaagtggaagcggcgatggcggagctgaattacattcccaaccgcgt |
| plasmid for | ggcacaacaactggcgggcaaacagtcgttgctgattggcgttgccacctccagtctggccctgcacgcgccgtcgcaaat |
| TXTL | tgtcgcggcgattaaatctcgcgccgatcaactgggtgccagcgtggtggtgtcgatggtagaacgaagcggcgtcgaagc |
| | ctgtaaagcggcggtgcacaatcttctcgcgcaacgcgtcagtgggctgatcattaactatccgctggatgaccaggatgc |
| | cattgctgtggaagctgcctgcactaatgttccggcgttatttcttgatgtctctgaccagacacccatcaacagtattat |
| | tttctcccatgaagacggtacgcgactgggcgtggagcatctggtcgcattgggtcaccagcaaatcgcgctgttagcggg |
| | cccattaagttctgtctcggcgcgtctgcgtctggctggctggcataaatatctcactcgcaatcaaattcagccgatagc |
| | ggaacgggaaggcgactggagtgccatgtccggttttcaacaaaccatgcaaatgctgaatgagggcatcgttcccactgc |
| | gatgctggttgccaacgatcagatggcgctgggcgcaatgcgcgccattaccgagtccgggctgcgcgttggtgcggatat |
| | ctcggtagtgggatacgacgataccgaagacagctcatgttatatcccgccgttaaccaccatcaaacaggattttcgcct |
| | gctggggcaaaccagcgtggaccgcttgctgcaactctctcagggccaggcggtgaagggcaatcagctgttgcccgtctc |
| | actggtgaaaagaaaaaccaccctggcgcccaatacgcaaaccgcctctccccgcgcgttggccgattcattaatgcagct |
| | ggcacgacaggtttcccgactggaaagcgggcaggctgcaaacgacgaaaactacgctttagtagcttaataactctgata |
| | gtgctagtgtagatccctactagagccaggcatcaaataaaacgaaaggctcagtcgaaagactgggcctttcgttttatc |
| | tgttgtttgtcggtgaacgctctctactagagtcacactggctcaccttcgggtgggcctttctgcgtttatatattgctt |
| | agaataatcgatctgcggccgcagagagtgtagcttacctagtcatcgaaagctttgctacagcggatagaattgtgagcg |
| | gataacaattgacattgtgagcggataacaagatactactagtgtctaacgaccttttaaatttctactgtttgtagatcg |
| | atgtgacatcaagtgctacggggtctaacgaccttttaaatttctactgtttgtagatcaaagcccgccgaaaggcgggct |
| | tttttttgtggatataccttactcgagttagccttgatagattgtctgattcgttaccaattatgacaacttgacggctac |
| | atcattcactttttcttcacaaccggcacggaactcgctcgggctggccccggtgcattttttaaatacccgcgagaaata |
| | gagttgatcgtcaaaaccaacattgcgaccgacggtggcgataggcatccgggtggtgctcaaaagcagcttcgcctggct |
| | gatacgttggtcctcgcgccagcttaagacgctaatccctaactgctggcggaaaagatgtgacagacgcgacggcgacaa |
| | gcaaacatgctgtgcgacgctggcgatatcaaaattgctgtctgccaggtgatcgctgatgtactgacaagcctcgcgtac |
| | ccgattatccatcggtggatggagcgactcgttaatcgcttccatgcgccgcagtaacaattgctcaagcagatttatcgc |
| | cagcagctccgaatagcgcccttccccttgcccggcgttaatgatttgcccaaacaggtcgctgaaatgcggctggtgcgc |
| | ttcatccgggcgaaagaaccccgtattggcaaatattgacggccagttaagccattcatgccagtaggcgcgcggacgaaa |
| | gtaaacccactggtgataccattcgcgagcctccggatgacgaccgtagtgatgaatctctcctggcgggaacagcaaaat |
| | atcacccggtcggcaaacaaattctcgtccctgatttttcaccaccccctgaccgcgaatggtgagattgagaatataacc |
| | tttcattcccagcggtcggtcgataaaaaaatcgagataaccgttggcctcaatcggcgttaaacccgccaccagatgggc |
| | attaaacgagtatcccggcagcaggggatcattttgcgcttcagccatacttttcatactcccgccattcagagaagaaac |
| | caattgtccatattgcatcagacattgccgtcactgcgtcttttactggctcttctcgctaaccaaaccggtaaccccgct |
| | tattaaaagcattctgtaacaaagcgggaccaaagccatgacaaaaacgcgtaacaaaagtgtctataatcacggcagaaa |
| | agtccacattgattatttgcacggcgtcacactttgctatgccatagcatttttatccataagattagcggatcctacctg |
| | acgctttttatcgcaactctctactgtttctccatatatcggatccttagtaaacctgcaggcactgcccatggacctcgg |
| | taccgaatagctagccggtaatgcattcgctagagctcctaaagcatgcgacctgcaaccggtctgtcacgtacgtcgcca |
| | ccgtcgacgtcgttcgtaagtagcctagataaataaaataatcagttaaccgcgagccccatgcgagagtagggaactgcc |
| | aggcatcaaataaaacgaaaggctcagtcgaaagactgggcctttcgttttatctgttgtttgtcggtgaacgctctcctg |
| | agtaggacaaatccgccgggagcggatttgaacgttgcgaagcaacggcccggagggtggcgggcaggacgcccgccataa |
| | actgccaggcatcaaattaagcagaaggccatcctgacggatggcctttttgcgtttctacaaactctgcggtaatacggt |
| | tatccacagaatcaggggataacgcaggaaagaacatgtgagcaaaaggccagcaaaaggccaggaaccgtaaaaaggccg |
| | cgttgctggcgtttttccataggctccgcccccctgacgagcatcacaaaaatcgacgctcaagtcagaggtggcgaaacc |
| | cgacaggactataaagataccaggcgtttccccctggaagctccctcgtgcgctctcctgttccgaccctgccgcttaccg |
| | gatacctgtccgcctttctcccttcgggaagcgtggcgctttctcatagctcacgctgtaggtatctcagttcggtgtagg |
| | tcgttcgctccaagctgggctgtgtgcacgaaccccccgttcagcccgaccgctgcgccttatccggtaactatcgtcttg |
| | agtccaacccggtaagacacgacttatcgccactggcagcagccactggtaacaggattagcagagcgaggtatgtaggcg |
| | gtgctacagagttcttgaagtggtggcctaactacggctacactagaaggacagtatttggtatctgcgctctgctgaagc |
| | cagttaccttcggaaaaagagttggtagctcttgatccggcaaacaaaccaccgctggtagcggtggtttttttgtttgca |
| | agcagcagattacgcgcagaaaaaaaggatctcaagaagatcctttgatcttttctacggggtctgacgctcagtggaacg |
| | aaaactcacgttaagggattttggtcatgagattatcaaaaaggatcttcacctagatccttttaaattaaaaatgaagtt |
| | ttaaatcaatctaaagtatatatgagtaaacttggtctgacagttaccaatgcttaatcagtgaggcacctatctcagcga |
| | tctgtctatttcgttcatccatagttgcctgactccccgtcgtgtagataactacgatacgggagggcttaccatctggcc |
| | ccagtgctgcaatgataccgcgagacccacgctcaccggctccagatttatcagcaataaaccagccagccggaagggccg |
| | agcgcagaagtggtcctgcaactttatccgcctccatccagtctattaattgttgccgggaagctagagtaagtagttcgc |
| | cagttaatagtttgcgcaacgttgttgccattgctacaggcatcgtggtgtcacgctcgtcgtttggtatggcttcattca |
| | gctccggttcccaacgatcaaggcgagttacatgatcccccatgttgtgcaaaaaagcggttagctccttcggtcctccga |
| | tcgttgtcagaagtaagttggccgcagtgttatcactcatggttatggcagcactgcataattctcttactgtcatgccat |
| | ccgtaagatgcttttctgtgactggtgagtactcaaccaagtcattctgagaatagtgtatgcggcgaccgagttgctctt |
| | gcccggcgtcaatacgggataataccgcgccacatagcagaactttaaaagtgctcatcattggaaaacgttcttcggggc |
| | gaaaactctcaaggatcttaccgctgttgagatccagttcgatgtaacccactcgtgcacccaactgatcttcagcatctt |
| | ttactttcaccagcgtttctgggtgagcaaaaacaggaaggcaaaatgccgcaaaaaagggaataagggcgacacggaaat |
| | gttgaatactcatactcttcctttttcaatattattgaagcatttatcagggttattgtctcatgagcggatacatatttg |
| | aatgtatttagaaaaataaacaaataggggttccgcgcacatttccccgaaaagtgccacctgacgtctaagaaaccatta |
| | ttatcatgacattaacctataaaaataggcgtatcacgaggcagaatttcagataaaaaaaatccttagctttcgctaagg |
| | atgatttctg |
| Cas12 GFp, | gaattctaaagatctggcacgtaagaggttccaactttcaccataatgaaacatactagagaaagaggagaaatactagat |
| RFP gRNA | ggtgaatgtgaaaccagtaacgttatacgatgtcgcagagtatgccggtgtctcttatcagaccgtttcccgcgtggtgaa |
| plasmid for | ccaggccagccacgtttctgcgaaaacgcgggaaaaagtggaagcggcgatggcggagctgaattacattcccaaccgcgt |
| TXTL | ggcacaacaactggcgggcaaacagtcgttgctgattggcgttgccacctccagtctggccctgcacgcgccgtcgcaaat |
| | tgtcgcggcgattaaatctcgcgccgatcaactgggtgccagcgtggtggtgtcgatggtagaacgaagcggcgtcgaagc |
| | ctgtaaagcggcggtgcacaatcttctcgcgcaacgcgtcagtgggctgatcattaactatccgctggatgaccaggatgc |
| | cattgctgtggaagctgcctgcactaatgttccggcgttatttcttgatgtctctgaccagacacccatcaacagtattat |
| | tttctcccatgaagacggtacgcgactgggcgtggagcatctggtcgcattgggtcaccagcaaatcgcgctgttagcggg |
| | cccattaagttctgtctcggcgcgtctgcgtctggctggctggcataaatatctcactcgcaatcaaattcagccgatagc |
| | ggaacgggaaggcgactggagtgccatgtccggttttcaacaaaccatgcaaatgctgaatgagggcatcgttcccactgc |
| | gatgctggttgccaacgatcagatggcgctgggcgcaatgcgcgccattaccgagtccgggctgcgcgttggtgcggatat |
| | ctcggtagtgggatacgacgataccgaagacagctcatgttatatcccgccgttaaccaccatcaaacaggattttcgcct |
| | gctggggcaaaccagcgtggaccgcttgctgcaactctctcagggccaggcggtgaagggcaatcagctgttgcccgtctc |
| | actggtgaaaagaaaaaccaccctggcgcccaatacgcaaaccgcctctccccgcgcgttggccgattcattaatgcagct |
| | ggcacgacaggtttcccgactggaaagcgggcaggctgcaaacgacgaaaactacgctttagtagcttaataactctgata |
| | gtgctagtgtagatccctactagagccaggcatcaaataaaacgaaaggctcagtcgaaagactgggcctttcgttttatc |
| | tgttgtttgtcggtgaacgctctctactagagtcacactggctcaccttcgggtgggcctttctgcgtttatatattgctt |
| | agaataatcgatctgcggccgcagagagtgtagcttacctagtcatcgaaagctttgctacagcggatagaattgtgagcg |
| | gataacaattgacattgtgagcggataacaagatactactagtgtctaacgaccttttaaatttctactgtttgtagataa |
| | agttcgtatggaaggttccgttgtctaacgaccttttaaatttctactgtttgtagatcactggagttgtcccaattcttg |
| | tgtctaacgaccttttaaatttctactgtttgtagatcaaagcccgccgaaaggcgggcttttttttgtggatatacctta |
| | ctcgagttagccttgatagattgtctgattcgttaccaattatgacaacttgacggctacatcattcactttttcttcaca |
| | accggcacggaactcgctcgggctggccccggtgcattttttaaatacccgcgagaaatagagttgatcgtcaaaaccaac |
| | attgcgaccgacggtggcgataggcatccgggtggtgctcaaaagcagcttcgcctggctgatacgttggtcctcgcgcca |
| | gcttaagacgctaatccctaactgctggcggaaaagatgtgacagacgcgacggcgacaagcaaacatgctgtgcgacgct |
| | ggcgatatcaaaattgctgtctgccaggtgatcgctgatgtactgacaagcctcgcgtacccgattatccatcggtggatg |
| | gagcgactcgttaatcgcttccatgcgccgcagtaacaattgctcaagcagatttatcgccagcagctccgaatagcgccc |
| | ttccccttgcccggcgttaatgatttgcccaaacaggtcgctgaaatgcggctggtgcgcttcatccgggcgaaagaaccc |
| | cgtattggcaaatattgacggccagttaagccattcatgccagtaggcgcgcggacgaaagtaaacccactggtgatacca |
| | ttcgcgagcctccggatgacgaccgtagtgatgaatctctcctggcgggaacagcaaaatatcacccggtcggcaaacaaa |
| | ttctcgtccctgatttttcaccaccccctgaccgcgaatggtgagattgagaatataacctttcattcccagcggtcggtc |
| | gataaaaaaatcgagataaccgttggcctcaatcggcgttaaacccgccaccagatgggcattaaacgagtatcccggcag |
| | caggggatcattttgcgcttcagccatacttttcatactcccgccattcagagaagaaaccaattgtccatattgcatcag |
| | acattgccgtcactgcgtcttttactggctcttctcgctaaccaaaccggtaaccccgcttattaaaagcattctgtaaca |
| | aagcgggaccaaagccatgacaaaaacgcgtaacaaaagtgtctataatcacggcagaaaagtccacattgattatttgca |
| | cggcgtcacactttgctatgccatagcatttttatccataagattagcggatcctacctgacgctttttatcgcaactctc |
| | tactgtttctccatatatcggatccttagtaaacctgcaggcactgcccatggacctcggtaccgaatagctagccggtaa |
| | tgcattcgctagagctcctaaagcatgcgacctgcaaccggtctgtcacgtacgtcgccaccgtcgacgtcgttcgtaagt |
| | agcctagataaataaaataatcagttaaccgcgagccccatgcgagagtagggaactgccaggcatcaaataaaacgaaag |
| | gctcagtcgaaagactgggcctttcgttttatctgttgtttgtcggtgaacgctctcctgagtaggacaaatccgccggga |
| | gcggatttgaacgttgcgaagcaacggcccggagggtggcgggcaggacgcccgccataaactgccaggcatcaaattaag |
| | cagaaggccatcctgacggatggcctttttgcgtttctacaaactctgcggtaatacggttatccacagaatcaggggata |
| | acgcaggaaagaacatgtgagcaaaaggccagcaaaaggccaggaaccgtaaaaaggccgcgttgctggcgtttttccata |
| | ggctccgcccccctgacgagcatcacaaaaatcgacgctcaagtcagaggtggcgaaacccgacaggactataaagatacc |
| | aggcgtttccccctggaagctccctcgtgcgctctcctgttccgaccctgccgcttaccggatacctgtccgcctttctcc |
| | cttcgggaagcgtggcgctttctcatagctcacgctgtaggtatctcagttcggtgtaggtcgttcgctccaagctgggct |
| | gtgtgcacgaaccccccgttcagcccgaccgctgcgccttatccggtaactatcgtcttgagtccaacccggtaagacacg |
| | acttatcgccactggcagcagccactggtaacaggattagcagagcgaggtatgtaggcggtgctacagagttcttgaagt |
| | ggtggcctaactacggctacactagaaggacagtatttggtatctgcgctctgctgaagccagttaccttcggaaaaagag |
| | ttggtagctcttgatccggcaaacaaaccaccgctggtagcggtggtttttttgtttgcaagcagcagattacgcgcagaa |
| | aaaaaggatctcaagaagatcctttgatcttttctacggggtctgacgctcagtggaacgaaaactcacgttaagggattt |
| | tggtcatgagattatcaaaaaggatcttcacctagatccttttaaattaaaaatgaagttttaaatcaatctaaagtatat |
| | atgagtaaacttggtctgacagttaccaatgcttaatcagtgaggcacctatctcagcgatctgtctatttcgttcatcca |
| | tagttgcctgactccccgtcgtgtagataactacgatacgggagggcttaccatctggccccagtgctgcaatgataccgc |
| | gagacccacgctcaccggctccagatttatcagcaataaaccagccagccggaagggccgagcgcagaagtggtcctgcaa |
| | ctttatccgcctccatccagtctattaattgttgccgggaagctagagtaagtagttcgccagttaatagtttgcgcaacg |
| | ttgttgccattgctacaggcatcgtggtgtcacgctcgtcgtttggtatggcttcattcagctccggttcccaacgatcaa |
| | ggcgagttacatgatcccccatgttgtgcaaaaaagcggttagctccttcggtcctccgatcgttgtcagaagtaagttgg |
| | ccgcagtgttatcactcatggttatggcagcactgcataattctcttactgtcatgccatccgtaagatgcttttctgtga |
| | ctggtgagtactcaaccaagtcattctgagaatagtgtatgcggcgaccgagttgctcttgcccggcgtcaatacgggata |
| | ataccgcgccacatagcagaactttaaaagtgctcatcattggaaaacgttcttcggggcgaaaactctcaaggatcttac |
| | cgctgttgagatccagttcgatgtaacccactcgtgcacccaactgatcttcagcatcttttactttcaccagcgtttctg |
| | ggtgagcaaaaacaggaaggcaaaatgccgcaaaaaagggaataagggcgacacggaaatgttgaatactcatactcttcc |
| | tttttcaatattattgaagcatttatcagggttattgtctcatgagcggatacatatttgaatgtatttagaaaaataaac |
| | aaataggggttccgcgcacatttccccgaaaagtgccacctgacgtctaagaaaccattattatcatgacattaacctata |
| | aaaataggcgtatcacgaggcagaatttcagataaaaaaaatccttagctttcgctaaggatgatttctg |

To prepare the plasmids for TXTL, a 20 mL culture of E. coli containing one of the plasmids was grown to high density, then isolated across five preparations using the Monarch Plasmid Miniprep Kit (New England Biolabs), eluting in a total of 200 μL nuclease-free H₂O. 200 μL of AMPure XP beads (Beckman Coulter) were then added to each combined miniprep and purified according to the manufacturer's instructions, eluting in a final volume of 20 μL in nuclease-free H₂O.

All anti-CRISPR candidate amplicons and subfragments were prepared using 100 L PCRs with either Q5, Phusion, or Taq LongAmp polymerase (all New England Biolabs), under various conditions to yield a strong band on an agarose gel such that the correct fragment length was greater than 95% of the fluorescence intensity on the gel. 100 μL of AMPure XP beads (Beckman Coulter) were then added to each reaction, and purified according to the manufacturer's instructions, eluting in a final volume of 10 μL in nuclease-free H₂O. The Cas12-containing amplicon was prepared the same way, except that the PCR was scaled to 500 μL and the resulting products were ethanol precipitated then dissolved in 100 μL of nuclease-free H₂O before the bead purification.

### TXTL Reactions

TXTL master mix was purchased from Arbor Biosciences and reactions were carried out in a total of 12 μL each. Each reaction contained 9 μL of TXTL master mix, 0.125 nM of each reporter plasmid, 1 nM of Cas12 amplicon, 2 nM of gRNA plasmid, 1 nM of genomic amplicon or Acr candidate plasmid, 1 μM of IPTG, 0.5 μM of anhydrotetracycline, and 0.1% arabinose. Additionally, we added 2 μM of annealed oligos containing six x sites as described in Marshall, et al. (2017).

The reactions were run at 29° C. in a TECAN Infinite Pro F200, measuring RFP (λₑₓ: 580 nm, λₑₘ: 620 nM) and GFP (λₑₓ: 485 nm, λₑₘ: 535 nm) fluorescence levels every three minutes for up to 10 hours. Fluorescence intensity was first normalized.

### Protein Purification

DNA encoding the sequences of the SpyCas9, MbCas12, AsCas12, and LbCas12 sequences were cloned into a custom vector containing, in order from the N-terminus: a 10× His tag, maltose binding protein (MBP), TEV protease cleavage site, the Cas12 sequence, and an optional C-terminal NLS sequence for proteins containing an NLS used in the gene editing assays. Protein purification proceeded largely as described in previous work (Jinek, 2012). Briefly, each plasmid containing Cas12 or Cas9 was grown in E. coli Rosetta2 cells overnight in Lysogeny Broth and subcultured in Terrific Broth until the OD₆₀₀ was between 0.6-0.8, after which protein production was induced with 375 μM IPTG and the cultures were grown at 16° C. for 16 hr. Cells were harvested and resuspended in Lysis Buffer (20 mM Tris-HCl pH 8.0, 500 mM NaCl, 10 mM imidazole, 0.5% Triton X-100, 1 mM TCEP, 1 mM PMSF, and Roche complete protease inhibitor cocktail), lysed by sonication, and purified using Ni-NTA superflow resin (Qiagen). The eluted proteins were cleaved with TEV protease overnight at 4° C., then purified on a Heparin HiTrap column using cation exchange chromatography with a linear KCl gradient. The protein-containing fractions were pooled and concentrated before application over a Superdex 200 size exclusion column (GE), exchanging the proteins into the final storage buffer containing 20 mM HEPES-HCl, pH 7.5, 200 mM KCl, 1 mM TCEP, and 10% glycerol.

### Nucleic Acid Purification for In Vitro Cleavage Experiments

Cas12 gRNA templates for in vitro transcription were prepared by amplifying three overlapping DNA oligos purchased from IDT to create a template containing a T7 RNA polymerase promoter, the gRNA sequence, and the Hepatitis 6 anti-genomic ribozyme. The templates were then transcribed and purified using standard methods.

To produce the DNA target for the dsDNA cleavage experiments, cells containing a minimal vector with the ColE1 origin and AmpR gene were grown and miniprepped using the Monarch Plasmid Miniprep Kit (NEB), eluting with water. The plasmid was then linearized using EcoRI, after which the enzyme was deactivated and the plasmid diluted to 50 nM in the 1× Cleavage Buffer for use in the in vitro cleavage experiments.

### In Vitro Cleavage Experiments

All dsDNA cleavage experiments were carried out in a 1× Cleavage Buffer that consisted of: 20 mM HEPES-HCl, pH 7.5, 150 mM KCl, 10 mM MgCl₂, 0.5 mM TCEP. gRNA sequences were first refolded by diluting the purified gRNA to 500 nM in 1× Cleavage Buffer, heating at 70° C. for 5 min then allowing to cool to room temperature. This was mixed with Cas12 protein diluted to 500 nM in 1× Cleavage Buffer at a 1:1 ratio and incubated at 37° C. for 10 min to form the RNP complex at 250 nM. To perform the cleavage reaction, a 9 uL mixture containing 5 nM of linearized plasmid and 0-1.25 μM anti-CRISPR candidate protein was prepared then incubated at 37° C. for 10 min before adding preformed RNP to 25 nM to start the reaction. The reaction was incubated 30 min at 37° C. before quenching with 2 μL of 6× Quench Buffer (30% glycerol, 1.2% SDS, 250 mM EDTA). The cleaved/uncleaved DNA was resolved on a 1% agarose gel prestained with SYBR Gold (Invitrogen).

### Mammalian Cell Culture

All mammalian cell cultures were maintained in a 37° C. incubator, at 5% CO₂. HEK293T (293FT; Thermo Fisher Scientific) human kidney cells and derivatives thereof were grown in Dulbecco's Modified Eagle Medium (DMEM; Corning Cellgro, #10-013-CV) supplemented with 10% fetal bovine serum (FBS; Seradigm #1500-500), and 100 Units/ml penicillin and 100 μg/ml streptomycin (100-Pen-Strep; Gibco #15140-122).

HEK293T and HEK-RT1 cells were tested for absence of mycoplasma contamination (UC Berkeley Cell Culture facility) by fluorescence microscopy of methanol fixed and Hoechst 33258 (Polysciences #09460) stained samples.

### Lentiviral Vectors

A lentiviral vector referred to as pCF525, expressing an EF1a-driven polycistronic construct containing a hygromycin B resistance marker, P2A ribosomal skipping element, and a fluorescence marker (mTagBFP2, mCherry) or an AcrVA (AcrV1, AcrV2, AcrV3), was loosely based on pCF204. In brief, to make the backbone more efficient, the f1 bacteriophage origin of replication and bleomycin resistance marker were removed. Within the provirus, the original expression cassette was replaced by the above described EF1a-driven HygroR-P2A-GOI (gene-of-interest) polycistronic constructs using custom oligonucleotides (IDT), gBlocks (IDT), standard cloning methods, and Gibson assembly techniques and reagents (NEB).

### Lentiviral Transduction

Lentiviral particles were produced in HEK293T cells using polyethylenimine (PEI; Polysciences #23966) based transfection of plasmids. HEK293T cells were split to reach a confluency of 70-90% at time of transfection. Lentiviral vectors were co-transfected with the lentiviral packaging plasmid psPAX2 (Addgene #12260) and the VSV-G envelope plasmid pMD2.G (Addgene #12259). Transfection reactions were assembled in reduced serum media (Opti-MEM; Gibco #31985-070). For lentiviral particle production on 6-well plates, 1 μg lentiviral vector, 0.5 μg psPAX2 and 0.25 μg pMD2.G were mixed in 0.4 mL Opti-MEM, followed by addition of 5.25 μg PEI. After 20-30 min incubation at room temperature, the transfection reactions were dispersed over the HEK293T cells. Media was changed 12 h post-transfection, and virus harvested at 36-48 h post-transfection. Viral supernatants were filtered using 0.45 μm cellulose acetate or polyethersulfone (PES) membrane filters, diluted in cell culture media if appropriate, and added to target cells. Polybrene (5 μg/mL; Sigma-Aldrich) was supplemented to enhance transduction efficiency, if necessary.

### Mammalian Gene Editing Inhibition Assay

For rapid and reliable assessment of genome editing efficiency of various CRISPR-Cas variants in mammalian cells, we previously established a fluorescence-based genome editing reporter cell line referred to as HEK-RT1. In brief, HEK293T human embryonic kidney cells were transduced at low-copy with the amphotropic pseudotyped RT3GEPIR-Ren.713 retroviral vector (C. Fellmann et al., Cell Rep. 5, 1704-13 (2013)), comprising an all-in-one Tet-On system enabling doxycycline-controlled GFP expression. Single clones were isolated and individually assessed. HEK-RT3-4 cells were derived from the clone that performed best in these tests. Since HEK-RT3-4 are puromycin resistant, monoclonal HEK-RT1 reporter cell lines were derived by transient transfection of HEK-RT3-4 cells with a pair of vectors encoding Cas9 and guide RNAs targeting the puromycin resistance gene, followed by identification and characterization of monoclonal derivatives that are puromycin sensitive and show doxycycline inducible and reversible GFP fluorescence. HEK-RT1 cells were derived from the clone that performed best in these tests.

To test the effect of genomic integration and expression of anti-CRISPR-Cas12a candidates (AcrVAs) in mammalian cells, HEK-RT1 were stably transduced with lentiviral vectors (pCF525) encoding AcrVA1, AcrVA2, AcrVA3, mTagBFP2 or mCherry. Transduced HEK-RT1 target cell populations were selected 48 h post-transduction using hygromycin B (400 μg/ml; Thermo Fisher Scientific #10687010). The derived polyclonal HEK-RT1-AcrVA1, HEK-RT1-AcrVA2, HEK-RT1-AcrVA3, HEK-RT1-mTagBFP2 and HEK-RT1-mCherry genome protection and editing reporter cell lines were then used to quantify gene editing inhibition by flow cytometry after transient transfection with CRISPR-Cas ribonucleoprotein complexes (RNPs) programmed with guide RNAs targeting the GFP reporter. RNP transfections were carried out using Lipofectamine 2000 (Thermo Fisher Scientific). Specifically, HEK-RT1 derived reporter cells were seeded in 24-well plates at 30% confluency 3-8 h prior to transfection. For each sample, the RNP complex was formed by mixing a 10 μL complexing solution containing 10 μM Cas9/Cas12 NLS-tagged protein, 12 μM eGFP-targeting gRNA, 20 mM HEPES pH 7.5, 0.6 mM TCEP, 160 mM KCl, and 8 mM MgCl₂ was incubated at 37° C. for 10 min. The RNPs were mixed with 25 μL Opti-MEM (Gibco #31985-070) and 1.6 μL Lipofectamine 2000 was mixed with 25 μL Opti-MEM in a separate tube. Diluted RNPs were added to the diluted Lipofectamine 2000, incubated 15 min at room temperature, and co-incubated with the respective reporter cells.

GFP expression in HEK-RT1 derived reporter cells was induced by 24 h of doxycycline (1 μg/ml; Sigma-Aldrich) treatment starting at 24 h post-transfection. Percentages of GFP-positive cells were quantified by flow cytometry (Attune NxT, Thermo Fisher Scientific), routinely acquiring 30,000 events per sample. Non-transfected and non-induced reporter cells were used for normalization.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

SEQUENCES

SEQ ID NO: 1
Cas12a amino acid sequence: MbCas12a
This MbCas12a sequence includes a C-terminal nuclear localization signal (NLS) and 3xHA
tag.
MLFQDFTHLYPLSKTVRFELKPIDRTLEHIHAKNFLSQDETMADMHQKVKVILDDYH
RDFIADMMGEVKLTKLAEFYDVYLKFRKNPKDDELQKQLKDLQAVLRKEIVKPIGN
GGKYKAGYDRLFGAKLFKDGKELGDLAKFVIAQEGESSPKLAHLAHFEKFSTYFTGF
HDNRKNMYSDEDKHTAIAYRLIHENLPRFIDNLQILTTIKQKHSALYDQIINELTASGL
DVSLASHLDGYHKLLTQEGITAYNTLLGGISGEAGSPKIQGINELINSHHNQHCHKSE
RIAKLRPLHKQILSDGMSVSFLPSKFADDSEMCQAVNEFYRHYADVFAKVQSLFDGF
DDHQKDGIYVEHKNLNELSKQAFGDFALLGRVLDGYYVDVVNPEFNERFAKAKTD
NAKAKLTKEKDKFIKGVHSLASLEQAIEHYTARHDDESVQAGKLGQYFKHGLAGVD
NPIQKIHNNHSTIKGFLERERPAGERALPKIKSGKNPEMTQLRQLKELLDNALNVAHF
AKLLTTKTTLDNQDGNFYGEFGVLYDELAKIPTLYNKVRDYLSQKPFSTEKYKLNFG
NPTLLNGWDLNKEKDNFGVILQKDGCYYLALLDKAHKKVFDNAPNTGKSIYQKMI
YKYLEVRKQFPKVFFSKEAIAINYHPSKELVEIKDKGRQRSDDERLKLYRFILECLKIH
PKYDKKFEGAIGDIQLFKKDKKGREVPISEKDLFDKINGIFSSKPKLEMEDFFIGEFKR
YNPSQDLVDQYNIYKKIDSNDNRKKENFYNNHPKFKKDLVRYYYESMCKHEEWEE
SFEFSKKLQDIGCYVDVNELFTEIETRRLNYKISFCNINADYIDELVEQGQLYLFQIYN
KDFSPKAHGKPNLHTLYFKALFSEDNLADPIYKLNGEAQIFYRKASLDMNETTIHRA
GEVLENKNPDNPKKRQFVYDIIKDKRYTQDKFMLHVPITMNFGVQGMTIKEFNKKV
NQSIQQYDEVNVIGIDRGERHLLYLTVINSKGEILEQCSLNDITTASANGTQMTTPYH
KILDKREIERLNARVGWGEIETIKELKSGYLSHVVHQISQLMLKYNAIVVLEDLNFGF
KRGRFKVEKQIYQNFENALIKKLNHLVLKDKADDEIGSYKNALQLTNNFTDLKSIGK
QTGFLFYVPAWNTSKIDPETGFVDLLKPRYENIAQSQAFFGKFDKICYNADKDYFEF
HIDYAKFTDKAKNSRQIWTICSHGDKRYVYDKTANQNKGAAKGINVNDELKSLFAR
HHINEKQPNLVMDICQNNDKEFHKSLMYLLKTLLALRYSNASSDEDFILSPVANDEG
VFFNSALADDTQPQNADANGAYHIALKGLWLLNELKNSDDLNKVKLAIDNQTWLN
FAQNRKRPAATKKAGQAKKKKGSYPYDVPDYAYPYDVPDYAYPYDVPDYA-
SEQ ID NO: 2
GF90 cand5, also referred to as AcrVA1
MSKAMYEAKERYAKKKMQENTKIDTLTDEQHDALAQLCAFRHKFHSNKDSLFLSE
SAFSGEFSFEMQSDENSKLREVGLPTIEWSFYDNSHIPDDSFREWFNFANYSELSETIQ
EQGLELDLDDDETYELVYDELYTEAMGEYEELNQDIEKYLRRIDEEHGTQYCPTGFA
RLR
SEQ ID NO: 3
GF122 cand9, also referred to as AcrVA2
MYEIKLNDTLIHQTDDRVNAFVAYRYLLRRGDLPKCENIARMYYDGKVIKTDVIDH
DSVHSDEQAKVSNNDIIKMAISELGVNNFKSLIKKQGYPFSNGHINSWFTDDPVKSKT
MHNDEMYLVVQALIRACIIKEIDLYTEQLYNIIKSLPYDKRPNVVYSDQPLDPNNLDL
SEPELWAEQVGECMRYAHNDQPCFYIGSTKRELRVNYIVPVIGVRDEIERVMTLEEV
RNLHK
SEQ ID NO: 4
GF122 cand10, also referred to as AcrVA3
MKIELSGGYICYSIEEDEVTIDMVEVTTKRQGIGSQLIDMVKDVAREVGLPIGLYAYP
QDDSISQEDLIEFYFSNDFEYDPDDVDGRLMRWS
Additional AcrVA1 proteins
SEQ ID NO: 5
>AKG19227.1 hypothetical protein AAX09_07405 [Moraxella bovoculi]
MYEAKERYAKKKMQENTKIDTLTDEQHDALAQLCAFRHKFHSNKDSLFLSESAFSG
EFSFEMQSDENSKLREVGLPTIEWSFYDNSHIPDDSFREWFNFANYSELSETIQEQGLE
LDLDDDETYELVYDELYTEAMGEYEELNQDIEKYLRRIDEEHGTQYCPTGFARLR
Additional AcrVA2 proteins
SEQ ID NO: 6
>AKG19228.1 hypothetical protein AAX09_07410 [Moraxella bovoculi]
MHHTIARMNAFNKAFANAKDCYKKMQAWHLLNKPKHAFFPMQNTPALDNGLAAL
YELRGGKEDAHILSILSRLYLYGAWRNTLGIYQLDEEIIKDCKELPDDTPTSIFLNLPD
WCVYVDISSAQIATFDDGVAKHIKGFWAIYDIVEMNGINHDVLDFVVDTDTDDNVY
VPQPFILSSGQSVAEVLDYGASLFDDDTSNTLIKGLLPYLLWLCVAEPDITYKGLPVS
REELTRPKHSINKKTGAFVTPSEPFIYQIGERLGSEVRRYQSIIDGEQKRNRPHTKRPHI
RRGHWHGYWQGTGQAKEFRVRWQPAVFVNSGRVSS
AcrVA2.2
SEQ ID NO: 7
>AKG12143.1 hypothetical protein AAX07_09320 [Moraxella bovoculi]
MHHTIARMNAFNKAFGNAKDCYKKMQAWHLNNKPKHIFSPLQNTLSLNEGLAALY
ELHGGKEDEHILSILCCLYLYGTWRNTLGIYQLDEEIIKDCKELPDDTPTSIFLNLPDW
CVYVDISSAKIATIDGGVAKHIKGFWAIYDNIEMHGVNHDVLNFIIDTDTDNNIYVPQ
SLILSSEMSVAESLDYGLTLFGYDESNELVKGMLPYLLWLCVAEPDITHKGLPVSREE
LTKPKHGINKKTGAFVTPSEPFIYQIGERLGGEVRRYQSLIDDEKNQNRHHTKRPHIR
RGHWHGYWQGTGQAKEFKVRWQPAVFVNSGV
Additional AcrVA3 proteins
SEQ ID NO: 8
>AKG19230.1 hypothetical protein AAX09_07420 [Moraxella bovoculi]
MVGKSKIDWQSIDWTKTNAQIAQECGRAYNTVCKMRGKLGKSHQGAKSPRKDKGI
SRPQPHLNRLEYQALATAKAKASPKAGRFETNTKAKTWTLKSPDNKTYTFTNLMHF
VRTNPHLFDPDDVVWRTKSNGVEWCRASSGLALLAKRKKAPLSWKGWRLISLTKD
NK
AcrVA3.2
SEQ ID NO: 9
>OOR90252.1 hypothetical protein B0181_04965 [Moraxella caviae]
MIAHQKNRRADWESVDWTKHNDEIAQLLSRHPDSVAKMRTKFGAQGMAKRKPRR
KYKVTRKAVPPPHTQELATAAAKISPKSGRYETNVNAKRWLIISPSGQRFEFSNLQHF
VRNHPELFAKADTVWKRQGGKRGTGGEYCNASNGLAQAARLNIGWKGWQAKIIK
G
SEQ ID NO: 10
AcrIE4-F7 (accession no. WP_064584002.1
MSTQYTYQQIAEDFRLWSEYVDTAGEMSKDEFNSLSTEDKVRLQVEAFGEEKSPKFS
TKVTTKPDFDGFQFYIEAGRDFDGDAYTEAYGVAVPTNIAARIQAQAAELNAGEWL
LVEHEA
AcrVA1 ortholog
SEQ ID NO: 11
CDA41774.1 Eubacterium eligens CAG: 72
MRMERNKEIATSANLADSLTEQQCDVLEWASDMRHVMHKNSEALYDVNAPKHKEI
KAFISDTHYSQNPNNLNKRLKDAGLPLIKWSFDDTRIPTNELAMILDNRRLEKSRKRQ
CIRTLEKANADIENYFAQIDAKYLTFYCPSGNKRVFSNQSKGVSVESRNVTIAEGYTI
MGIESLKNNIAYLLHVYAGQISIEDIVAYVNEDIENRIYHMDASMAPQSVHKATNAL
VETGYIKPDTKELIYINLTKRGDAFVGSYCGTLNKIAASLSILPFNKAHKNDIVYYSYL
IGWQRAKRELKPLIPKTVDFISNKIKEKQEMMYTGDDSNYAVEMEQTIIQSMNINSLP
VVYEVKKGTYVIAEITTLFGKINVSIINSLFVGSAYTLTIPQYQYTAIIHMADNIDYGKI
PYEVQKQLKAVVPVLMKLLQ
AcrVA1 orthologs
SEQ ID NO: 12
WP_003671752.1 Moraxella catarrhalis
MHRTIARMHKFNKEFTNAKECYKKMQQAYLASKNKFAFFPMQHASLLDMSTAIAY
EQTRSDPFSEKGVNALKTLNQLYLFGTWRYTLGIYCLDDEIIKDSKAIPDDTPTSIFLN
LPEWCVYLDIASAKIAITQDNKTRHIKGFWAVYDLIEYNSKPQKAINFIIDTDSDDDIY
LPLTLILDDDMTVEQSLSYADNKIGDGGSNELIKVLLPYLLWLCVAEPEIMHKGEPVS
RANLDKPKYQTNKKTGVFIPPSEPFIYEVGSRLGGEIRHYQEQIEQGKHRQTSKKRPH
IRRGHWHGHWHGTGQAKEFKIKWQPAIFVNSGV
SEQ ID NO: 13
AKI27019.1 Moraxella phage Mcat2
MKMHHTIARMQKFNKEFTNAKACYKKMQQAYLTSKNKFAFFPMQHASLLDMSTAI
AYEQTRSDPFSEKGVNALKTLNQLYLFGTWRYTLGIYCLDDEIIKDSKAIPDDTPTSIF
LNLPEWCVYLDIASAKIAITQDNKTRHIKGFWAVYDLIEYNSKPQKAINFIIDTDSDD
DIYLPLTLILDDDMTVMQSLSYADNKIGDGGSNELIKVLLPYLLWLCVAEPEIMHKG
EPVSRANLDKPKYQTNKKTGVFIPPSEPFIYEVGSRLGGEIRHYQEQIEQGKHRQTSK
KRPHIRRGHWHGHWHGTGQAKEFKIKWQPAIFVNSGV
SEQ ID NO: 14
OBX64325.1 Moraxella osloensis
MIKDKDGNCIHGYDCYLAFNRKYPEAKELYKKLAEDQKNNPSKNGVYTTQQRIFQI
SDFLAEKTPSIQRLIADPRLYNPEKEPYTSFLSYVNGMPMFSAWRNSLDIYKIDPEIFE
EMIKSPIPKDTPCEVFKRLPNFCVYVEMPRPTKFNELLMGNLNHLDKSFIVNGFWAY
LGIEPNLHGNKNIQLNICLDYSSDIVQGNFDFLSMVIKEGLTVEEATELVFKQYDGNIE
TAKQDQRALFALLPILLWLCAEQPDITNIKDEPVTHEQLQQPKGSIHKKTGLFVPPNS
PTYYNLGKRLGGEIRQYQELIKQDEKDRPTASKRPHIRKGHWHGYWKGTTGNKVFT
PKWLSAIFVGFN
SEQ ID NO: 15
EGE16485.1 Moraxella catarrhalis BC1
MIEYNSKPQKAINFIIDTDSDDDIYLPLTLILDDDMTVEQSLSYADNNIGDGGSNELIKI
LLPYLLWLCVAEPEIMHKGEPVSRANLDKPKYQTNKKTGVFIPPSEPFIYEVGSRLGG
EIRHYQEQIEQGKHRQTSKKRPHIRRGHWHGHWHGTGQAKEFKIKWQPAIFVNAGV
SEQ ID NO: 16
EGE16486.1 Moraxella catarrhalis BC1
MHRTIARMHKFNKEFTNAKECYKKMQQAYLASKNKFAFFPMQHASLLDMSTAIAY
EQTRSDPFSEKGVNALKTLNQLYLFGTWRYTLGIYCLDSEIIKDSKAIPNDTPTSIF
SEQ ID NO: 17
WP_065262896.1 Moraxella osloensis
MIKDKDGNCIHGYDCYLAFNRKYPEAKELYKKLAEDQKNNPSKNGVYTTQQRIFQI
SDFLAEKTPSIQRLIADPRLYNPEKEPYTSFLSYVNGMPMFSAWRNSLDIYKIDPEIFE
EMIKSPIPKDTPCEVFKRLPNFCVYVEMPRPTKFNELLMGNLNHLDKSFIVNGFWAY
LGIEPNLHGNKNIQLNICLDYSSDIVQGNFDFLSMVIKEGLTVEEATELVFKQYDGNIE
TAKQDQRALFALLPILLWLCAEQPDITNIKDEPVTHEQLQQPKGSIHKKTGLFVPPNS
PTYYNLGKRLGGEIRQYQELIKQDEKDRPTASKRPHIRKGHWHGYWKGTTGNKVFT
PKWLSAIFVGFN
SEQ ID NO: 18
WP_065262429.1 Moraxella osloensis
MLPYMTPFERYQAFVKTYPEAKETFKTMQAWYVANKPKNGIFVPSGNLYTMSPML
MKLVASKSKLAQSFTTMTDNDRLHLNYFWGLSLFGTWRYTLGVYQINDNLFDTLV
KSPIPDDTPTSIFDKLPEWCVYIAFPEGKAINIKFNNGFADYEAFIFGFWVKLDTQNLT
TSEGEQKIRVINFHLNLQTGIDNVFSNLQPLQLMIADDLSIKEAMQKHAKMVFEAYT
PNHDFIVTQQNAKQDYDLTNKLLSLLLMLCAEAPDISKITGEPITKIELGKPKYTVNK
RTGVFIPPQAPFLYEIGRRLGGDIKTTNDQLKNAGQGSGKGRRPHIRNAHYHGYWIG
TGQNKQFKLNWIAPIFVNG
SEQ ID NO: 19
ATR79575.1 Moraxella osloensis
MTEEKYGGDPFEFMHAVNREFIDRKKDFNILAENYIDRHKTRGKQAYIDMGYLMGY
IAHKYKINTHFQSEIPLGGVRDGSTVGKDAFSLAMFATWRLKPYVFEIDDDLFEQIKK
SPIPFESPVSIFDNLPAWAVYVQLSNHELSIYTPAHEIIKLKCYGFWAYKAYSGEQLW
LYMYPHVSQDDMTKTVNIQKFLPTSFLIINEKLDLFESLKKALEKMMDKKQEQHITP
EIWDMHLNNSRLFLSALLLLCVERPQIEDSSLNEVDIASLSHLPPIHPKTKRFIAPNEPT
KFFIGRRLGGQIRAFKAQESKGMPTGVTMQPHVRQAHWHGYRYGEGRKQFKLTFLP
PIFVNMHAEDNLEERD
AcrVA3 and AcrVA3.2 orthologs
SEQ ID NO: 20
WP_077553337.1 Rodentibacter ratti
MRRIDWHSVDWTKNNRQLADELGKAYDTVAKKRWELGQSGKAKDRAVRVDKGV
SKTTCVPSPQQQRYATEMAKISPKSGKFETNIHSKKYKITSPDNQVFVITNLYQFVRD
NKGLFLPTDVIFKRQGGTRGTGGEYCNATSGLLYISKHKTRTWKGWKCELLDSK
SEQ ID NO: 21
KXU39010.1 Ventosimonas gracilis
MVNQIKRRIKAASWEAMDWTKSNSQIAAETGKAYDTVAKRRVALGKSGMALQRSP
RKDLKQLIARLQTPEMREKSKANQPLATQAAKASPKAGRGIDNVHAEDWHLLSPTG
DSYKVRNLYEFVRANAHLFPPADVVWKRQGGARGTGGEYCNATAGILNIKGGKAK
SWKGWRMV
SEQ ID NO: 22
KKZ55830.1 Haemophilus haemolyticus
MDTVSRRRKQLARDTLLHQFRDWQNVDWSKTNKQLAIELGKSYDTVAKHRYQLG
HGGEAKEREVRSDKGISKTTNIPSPELQKYATEQAQKSPNSGKFETNIHAKKWRITSP
DNRVFVATNLYQFVRDNTALFLPSDVIFKRTGGKRGTGGEYCNATSGLLQAAASGR
LWKGWKCKQIKKDNHEL
SEQ ID NO: 23
WP_109133530.1 Aggregatibacter sp. Me1o68
MSKIDWRAVDWSKRTIDLSRELNRTAKTVSDNRAKYAPETLKSHKNIDWLKIDWLK
TTVQIAKELKVDFCTVAKARKKYAPETVIITPDWGKVDWTKNNRQLSQELGKSYNT
VAKHRYQLGHSGEAKEREPKSNKGAPNPKMSHGRINQPKATAAAKNSPKSGKFETN
IHAKKWRITSPDNQVFIVTNLYQFVRDHTHLFLPGDVIFKRTGGKRGTGGEYCNATN
GLANAYTTKRGLWKGWRCKQIKEDKKR
SEQ ID NO: 24
WP_050541864.1 Haemophilus haemolyticus
MSKIDWRTIDWSKRTIDLSRELNRTIKTVSDNRAKYAPETLKSHKNIDWLKIDWLKT
TVQIAKELKVGFCAVAKARKKYAPETVITPNWDEVDWTKNNRQLAQELGKSYNTV
AKKRCQLKQSGKAKERSVRIDKGQKKPQMAFGVVNQPLATKAAKTSPKSGKFETNI
HAKKWRITSPDNRVFVATNLYQFVRDNTALFLPGDVIFKRTGGKRGTGGEYCNATS
GLLQAAASGRLWKGWKCKQIKKDNHEL
SEQ ID NO: 25
WP_052749733.1 Haemophilus haemolyticus
MSKIDWASVDWSMRSIDIARLLDVTIDTVSRRRKQLARDTLLHQFRDWQNVDWSKT
NKQLAIELGKSYDTVAKHRYQLGHGGEAKEREVRSDKGISKTTNIPSPELQKYATEQ
AQKSPNSGKFETNIHAKKWRITSPDNRVFVATNLYQFVRDNTALFLPSDVIFKRTGG
KRGTGGEYCNATSGLLQAAASGRLWKGWKCKQIKKDNHEL
SEQ ID NO: 26
AHG75457.1 Mannheimia varigena
MSRATKINWSELDWSKSTLELSKMLNVAGNFVSLIKRRKYAPNTVRQKKAVDWSAI
DWSKSTSDIAKQIGWSVANVSQKRKKYAPDTMGNLRNVGKYKRKVKPTVLKAPNG
DILYMDSIKDFVIEYAHLFEAKHLISKNKKSGHIRQYCLAESALSSLRQKRVKKWQG
WSLYEGFEEQSKLKRIDWDNVDWTKNNDQLAKELNRAYDTVAKKRYLLGKSGMA
TSRKEKADKGQKNPKKAIGAIKTQPIAKEWAKKSQKSGKFETNVHAKRWRLTREDG
KCWEFTNLYHFVRTHTELFLPNDTVWKRTGGKRGTGGEYCNATSGLLNACRSRSK
KWKGWKIEKIEN
SEQ ID NO: 27
WP_109064402.1 Aggregatibacter sp. Melo83
MSKIDWRAVDWSKRTIDLSRELNRTAKTVSDNRAKYAPETLKSHKNIDWLKIDWLK
TTVQIAKELKVDFCTAAKARKKYAPETVIITPDWDKVDWTKSNRQLSQELGKSYNT
VAKHRYQLGHSGEAKEREPKSNKGVPNPKMSHGRINQPKATEAAKNSPKSGKFETN
IHAKKWRITSPDNQVFVATNLYQFVRDHTHLFLPGDVIFKRTGGKRGTGGEYCNAT
NGLANASTTKREMWKGWKCEKIKEGK
SEQ ID NO: 28
OFO25420.1 Neisseria sp. HMSC056A03
MPKYDWDKIDWRLSNHEIAAILQCSYDTVASKRYRLKVGKATKPKTRSDKGISRTT
YLPPKEQQRRAVEAAKASPKAGRGETNCHAKRWRLTDPYGKQYEFSNLHHFIRCNN
NLFTRKDVVWKRTGSNGGGEYCNASAGLQNVVAGKSPAWKGWEIEEITND
SEQ ID NO: 29
WP_083950388.1 Serratia ficaria
MRLLICLTLSRSRKTGALPMAGRINSRAEAEAYVAGDLVECLECGKKFAFLPVHIKR
MHGLNAEEYRERYNIPAGIPLAGKAYREMQRQKLVAMQKDGILDYSHLPKAEKAA
RRAGRGDKRDFDRQSQSHIMKLVNESGRAYRKTKSLFTPTAADNSIARVGPSYEQIE
FIKNNAHKMSASEMQRELGISRKVIKRRADKLGLSLLKGKPPVSKPTLDWGSVDWS
KSNKEIAASLGASYSAVKAMRRRLGVGPGKRAPMSNKGVKRNYSPEHLALIKKNAE
KMRLAALSSSKISRTEHNIHAKKWTLVSPDGEVYRVVNLHNFIRENTELFNPEDVVW
KLNGEEAEEGSRLWCRASQGIRSIKQRSVESWKGWKLLNPEDDEP
SEQ ID NO: 30
ATG94602.1 Acidovorax citrulli
MRKLADWAALDWAKPNAALAAEVGASVHTVAKRRTQHGVPMASPTWTRPDVAAI
NRRPERRAQSARTQPAATAAAKQSPAAGRGPDNVHALDWVLVSPSGERHQVRNLY
DFVRSHSALFAEADVVWKRTGGKRGTGGEWCNATAGILNIKGGRAKSWKGWTLA
Q
SEQ ID NO: 31
SDP29509.1 Acidovorax cattleyae
MRKLADWESLDWAKSNAVLAVEVGASIHTVAKRRTQHGVPTDSPTWKRPDVAAIN
QRPERRAQSARTQPAATAAARQSPAAGRGPENVHAVDWVLVSPSGERHQVRNLYD
FVRSHAALFAEADVAWKRTGGKRGTGGEWCNATAGILNIKGGRAKSWKGWTLAQ
SEQ ID NO: 32
GF90 cand5 ortholog
>WP_046701302.1 hypothetical protein [Moraxella bovoculi]
MYEAKERYAKKKMQENTKIDTLTDEQHDALAQLCAFRHKFHSNKDSLFLSESAFSG
EFSFEMQSDENSKLREVGLPTIEWSFYDNSHIPDDSFREWFNFANYSELSETIQEQGLE
LDLDDDETYELVYDELYTEAMGEYEELNQDIEKYLRRIDEEHGTQYCPTGFARLR
SEQ ID NO: 33
GF90 cand5 ortholog
>WP_046697118.1 hypothetical protein [Moraxella bovoculi]
MSETIQEQGLELDLDDDATYELVYDELYTEAMAEYEKLNQDIEKYLRRIDEEYGTQY
CPTGFARLR
SEQ ID NO: 34
GF90 cand5 ortholog
>CDA41774.1 putative uncharacterized protein [Eubacterium eligens CAG:72]
DSLTEQQCDVLEWASDMRHVMHKNSEALYDVNAPKHKEIKAFISDTHYSQNPNNL
NKRLKDAGLPLIKWSFDDTRIPTNELAMILDNRRLEKSRKRQCIRTLEKANADIENYF
AQIDAKYLTFYCPSGNKRV
SEQ ID NO: 35
GF90 cand5 ortholog
>OLA16786.1 hypothetical protein BHW24_02870 [[Eubacterium]eligens]
DSLTEQQCDVLEWASDMRHVMHKNSEALYDVNAPKHKEIKAFISDTHYSQNPNNL
NKRLKDAGLPLIKWSFDDTRIPTNELAMILDNRRLEKSRKRQCIRTLEKANADIENYF
AQIDAKYLTFYCPSGNKRV
SEQ ID NO: 36
GF90 cand5 ortholog
>WP_012740477.1 hypothetical protein [[Eubacterium]eligens]
DSLTEQQCDVLEWASDMRHVMHKNSEALYDVNAPKHKEIKAFISDTHYSQNPNNL
NKRLKDAGLPLIKWSFDDTRIPTNELAMILDNRRLEKSRKRQCIRTLEKANADIENYF
AQIDAKYLTFYCPSGNKRV
SEQ ID NO: 37
GF90 cand5 ortholog
>PWN29770.1 hypothetical protein BDZ90DRAFT_273637 [Jaminaea rosea]
KLDLREDEEGTVGLVDGRVRDEMRHEYEEMDQEVERQEVKIDEEEGTRILST
SEQ ID NO: 38
GF122 cand9 ortholog
>WP_046701923.1 hypothetical protein [Moraxella bovoculi]
MYEIKLNDTLIHQTDDRVNAFVAYRYLLRRGDLPKCENIARMYYDGKVIKTDVIDH
DSVHSDEQAKVSNNDIIKMAISELGVNNFKSLIKKQGYPFSNGHINSWFTDDPVKSKT
MHNDEMYLVVQSLIRACKIKEIDLYTEQLYNIIKSLPYDKRPNVVYSDQPLDPNNLD
LSEPELWAEQVGECMRYAHNDQPCFYIGSTKRELRVNYIVPVIGVRDEIERVMTLEE
VRNLHK
AcrVA6
SEQ ID NO: 39
VA6: >OOR90226.1 hypothetical protein B0181_04970 [Moraxella caviae]
MNKKSISQRVRRINNPKDKLALVQEWVSQRQSDFFSAFEQLEYAVGVDDLQQIHEA
MDKIKDIAIKNYKAMPNIAEAMLVSKHYTVDLDEYEQEK
SEQ ID NO: 40
AcrIE5 (accession no. WP_074973300.1)
MSNDRNGIINQIIDYTGTDRDHAERIYEELRADDRIYFDDSVGLDRQGLLIREDVDLM
AVAAEIE
SEQ ID NO: 41
AcrIE6 (accession no. WP_087937214.1)
MNNDTEVLEQQIKAFELLADELKDRLPTLEILSPMYTAVMVTYDLIGKQLASRRAELI
EILEEQYPGHAADLSIKNLCP
SEQ ID NO: 42
AcrIE7 (accession no. WP_087937215.1)
MIGSEKQVNWAKSIIEKEVEAWEAIGVDVREVAAFLRSISDARVIIDNRNLIHFQSSGI
SYSLESSPLNSPIFLRRFSACSVGFEEIPTALQRIRSVYTAKLLEDE
SEQ ID NO: 43
AcrIF11 (accession no. WP_038819808.1)
MSMELFHGSYEEISEIRDSGVFGGLFGAHEKETALSHGETLHRIISPLPLTDYALNYEI
ESAWEVALDVAGGDENVAEAIMAKACESDSNDGWELQRLRGVLAVRLGYTSVEM
EDEHGTTWLCLPGCTVEKI
SEQ ID NO: 44
AcrIF11.2 (accession no. EGE18857.1)
MTTLYHGSHENTAPVIKIGFAAFLPADNVFDGIFANGDKNVARSHGDFIYAYEVDSI
ATNDDLDCDEAIQIIAKELYIDEETAAPIAEAVAYEESLAEFEEHIMPRSCGDCADFG
WEMQRLRGVIARKLGFDAVECVDEHGVSHLIVNANIRGSIA
SEQ ID NO: 45
AcrIF12 (accession no. ABR13388.1)
MAYEKTWHRDYAAESLIKRAETSRWTQDANLEWTQLALECAQVVHLARQVGEELG
NEKIIGIADTVLSTIEAHSQATYRRPCYKRITTAQTHLLAVTLLERFGSARRVANAVW
QLTDDEIDQAKA
SEQ ID NO: 46
AcrIF13 (accession no. EGE18854.1)
MKLLNIKINEFAVTANTEAGDELYLQLPHTPDSQHSINHEPLDDDDFVKEVQEICDEY
FGKGDRTLARLSYAGGQAYDSYTEEDGVYTTNTGDQFVEHSYADYYNVEVYCKAD
LV
SEQ ID NO: 47
AcrIF14 (accession no. AKI27193.1)
MKKIEMIEISQNRQNLTAFLHISEIKAINAKLADGVDVDKKSFDEICSIVLEQYQAKQI
SNKQASEIFETLAKANKSFKIEKFRCSHGYNEIYKYSPDHEAYLFYCKGGQGQLNKLI
AENGRFM
SEQ ID NO: 48
Orf1(Pse)(accession no. SDJ61947.1)
MGVVVVLIIRLKARWSLHLERKLGEAGKAGIWEFHRSESSYTTDGRTTFRNAALRPA
EPKEGQTVEVFICSDSREPEEQWRAVGEGVARYE
SEQ ID NO: 49
Orf2(Pse)(accession no. WP_084336955.1)
MLSVLFFWLYFYALFFIRFASSNKRARGRGMQRPALVSIALEWGMRRELMSRSFTTR
IDHLQEVSRLGRGVARLRLGHSGRNLMPLILERRDGTGLTLKLDPKADPDEALRQLA
RGGIHVRVYSKYGERMRVVVDAPQAISILRDELVDRE
SEQ ID NO: 50
Aca4 (accession no. ABR13385.1)
MTEEQFSALAELMRLRGGPGEDAARLVLVNGLKPTDAARKTGITPQAVNKTLSSCR
RGIELAKRVFT
SEQ ID NO: 51
AcrIC1 (accession no. AKG19229.1)
MNNLKKTAITHDGVFAYKNTETVIGSVGRNDIVMAIDATHGEFNDKNFIIYADTNGN
PIYLGYAYLDDNNDAHIDLAVGACNEDDDFDEKEIHEMIAEQMELAKRYQELGDTV
HGTTRLAFDDDGYMTVRLDQQAYPDYRPENDDKHIMWRALALTATGKELEVFWL
VEDYEDEEVNSWDFDIADDWREL
SEQ ID NO: 52
Orf1(Mor)(accession no. EGE18856.1)
MSKNKTPDYVLRANANYRKKHTTNKSLQLHNEKDADIIQALQNETKSFNALMKDIL
RNHYNLNQNQ
SEQ ID NO: 53
Orf2(Mor)(accession no. AKG19231.1)
MNNPKTPEYTRKAIRAYEKNLVRKSVTFDVRKDDDMELLKMIEQDGRTFAQIARTA
LLEHLQK
SEQ ID NO: 54
For experiments in human cells (FIG. 9), the following fusion sequence for nuclear
localization signal and 3xHA tag was added to the C-terminus of each protein of Example 1:
GSGGGGSGPKKKRKVSSGYPYDVPDYAYPYDVPDYAYPYDVPDYA
SEQ ID NO: 55
MbCas12a DNA Sequence (pTE4495):
This is the MbCas12a (237) sequence cloned into pTN7C130 and expressed in PAO1 for
phage-targeting assays. This sequence is human codon-optimized and include a C-terminal
nuclear localization signal (NLS) and 3xHA tag.
ATGCTGTTCCAGGACTTTACCCACCTGTATCCACTGTCCAAGACAGTGAGATTTG
AGCTGAAGCCCATCGATAGGACCCTGGAGCACATCCACGCCAAGAACTTCCTGT
CTCAGGACGAGACAATGGCCGATATGCACCAGAAGGTGAAAGTGATCCTGGACG
ATTACCACCGCGACTTCATCGCCGATATGATGGGCGAGGTGAAGCTGACCAAGC
TGGCCGAGTTCTATGACGTGTACCTGAAGTTTCGGAAGAACCCAAAGGACGATG
AGCTGCAGAAGCAGCTGAAGGATCTGCAGGCCGTGCTGAGAAAGGAGATCGTGA
AGCCCATCGGCAATGGCGGCAAGTATAAGGCCGGCTACGACAGGCTGTTCGGCG
CCAAGCTGTTTAAGGACGGCAAGGAGCTGGGCGATCTGGCCAAGTTCGTGATCG
CACAGGAGGGAGAGAGCTCCCCAAAGCTGGCCCACCTGGCCCACTTCGAGAAGT
TTTCCACCTATTTCACAGGCTTTCACGATAACCGGAAGAATATGTATTCTGACGA
GGATAAGCACACCGCCATCGCCTACCGCCTGATCCACGAGAACCTGCCCCGGTTT
ATCGACAATCTGCAGATCCTGACCACAATCAAGCAGAAGCACTCTGCCCTGTAC
GATCAGATCATCAACGAGCTGACCGCCAGCGGCCTGGACGTGTCTCTGGCCAGC
CACCTGGATGGCTATCACAAGCTGCTGACACAGGAGGGCATCACCGCCTACAAT
ACACTGCTGGGAGGAATCTCCGGAGAGGCAGGCTCTCCTAAGATCCAGGGCATC
AACGAGCTGATCAATTCTCACCACAACCAGCACTGCCACAAGAGCGAGAGAATC
GCCAAGCTGAGGCCACTGCACAAGCAGATCCTGTCCGACGGCATGAGCGTGTCC
TTCCTGCCCTCTAAGTTTGCCGACGATAGCGAGATGTGCCAGGCCGTGAACGAGT
TCTATCGCCACTACGCCGACGTGTTCGCCAAGGTGCAGAGCCTGTTCGACGGCTT
TGACGATCACCAGAAGGATGGCATCTACGTGGAGCACAAGAACCTGAATGAGCT
GTCCAAGCAGGCCTTCGGCGACTTTGCACTGCTGGGACGCGTGCTGGACGGATA
CTATGTGGATGTGGTGAATCCAGAGTTCAACGAGCGGTTTGCCAAGGCCAAGAC
CGACAATGCCAAGGCCAAGCTGACAAAGGAGAAGGATAAGTTCATCAAGGGCG
TGCACTCCCTGGCCTCTCTGGAGCAGGCCATCGAGCACTATACCGCAAGGCACG
ACGATGAGAGCGTGCAGGCAGGCAAGCTGGGACAGTACTTCAAGCACGGCCTGG
CCGGAGTGGACAACCCCATCCAGAAGATCCACAACAATCACAGCACCATCAAGG
GCTTTCTGGAGAGGGAGCGCCCTGCAGGAGAGAGAGCCCTGCCAAAGATCAAGT
CCGGCAAGAATCCTGAGATGACACAGCTGAGGCAGCTGAAGGAGCTGCTGGATA
ACGCCCTGAATGTGGCCCACTTCGCCAAGCTGCTGACCACAAAGACCACACTGG
ACAATCAGGATGGCAACTTCTATGGCGAGTTTGGCGTGCTGTACGACGAGCTGG
CCAAGATCCCCACCCTGTATAACAAGGTGAGAGATTACCTGAGCCAGAAGCCTT
TCTCCACCGAGAAGTACAAGCTGAACTTTGGCAATCCAACACTGCTGAATGGCTG
GGACCTGAACAAGGAGAAGGATAATTTCGGCGTGATCCTGCAGAAGGACGGCTG
CTACTATCTGGCCCTGCTGGACAAGGCCCACAAGAAGGTGTTTGATAACGCCCCT
AATACAGGCAAGAGCATCTATCAGAAGATGATCTATAAGTACCTGGAGGTGAGG
AAGCAGTTCCCCAAGGTGTTCTTTTCCAAGGAGGCCATCGCCATCAACTACCACC
CTTCTAAGGAGCTGGTGGAGATCAAGGACAAGGGCCGGCAGAGATCCGACGATG
AGCGCCTGAAGCTGTATCGGTTTATCCTGGAGTGTCTGAAGATCCACCCTAAGTA
CGATAAGAAGTTCGAGGGCGCCATCGGCGACATCCAGCTGTTTAAGAAGGATAA
GAAGGGCAGAGAGGTGCCAATCAGCGAGAAGGACCTGTTCGATAAGATCAACG
GCATCTTTTCTAGCAAGCCTAAGCTGGAGATGGAGGACTTCTTTATCGGCGAGTT
CAAGAGGTATAACCCAAGCCAGGACCTGGTGGATCAGTATAATATCTACAAGAA
GATCGACTCCAACGATAATCGCAAGAAGGAGAATTTCTACAACAATCACCCCAA
GTTTAAGAAGGATCTGGTGCGGTACTATTACGAGTCTATGTGCAAGCACGAGGA
GTGGGAGGAGAGCTTCGAGTTTTCCAAGAAGCTGCAGGACATCGGCTGTTACGT
GGATGTGAACGAGCTGTTTACCGAGATCGAGACACGGAGACTGAATTATAAGAT
CTCCTTCTGCAACATCAATGCCGACTACATCGATGAGCTGGTGGAGCAGGGCCA
GCTGTATCTGTTCCAGATCTACAACAAGGACTTTTCCCCAAAGGCCCACGGCAAG
CCCAATCTGCACACCCTGTACTTCAAGGCCCTGTTTTCTGAGGACAACCTGGCCG
ATCCTATCTATAAGCTGAATGGCGAGGCCCAGATCTTCTACAGAAAGGCCTCCCT
GGACATGAACGAGACAACAATCCACAGGGCCGGCGAGGTGCTGGAGAACAAGA
ATCCCGATAATCCTAAGAAGAGACAGTTCGTGTACGACATCATCAAGGATAAGA
GGTACACACAGGACAAGTTCATGCTGCACGTGCCAATCACCATGAACTTTGGCGT
GCAGGGCATGACAATCAAGGAGTTCAATAAGAAGGTGAACCAGTCTATCCAGCA
GTATGACGAGGTGAACGTGATCGGCATCGATCGGGGCGAGAGACACCTGCTGTA
CCTGACCGTGATCAATAGCAAGGGCGAGATCCTGGAGCAGTGTTCCCTGAACGA
CATCACCACAGCCTCTGCCAATGGCACACAGATGACCACACCTTACCACAAGAT
CCTGGATAAGAGGGAGATCGAGCGCCTGAACGCCCGGGTGGGATGGGGCGAGA
TCGAGACAATCAAGGAGCTGAAGTCTGGCTATCTGAGCCACGTGGTGCACCAGA
TCAGCCAGCTGATGCTGAAGTACAACGCCATCGTGGTGCTGGAGGACCTGAATTT
CGGCTTTAAGAGGGGCCGCTTTAAGGTGGAGAAGCAGATCTATCAGAACTTCGA
GAATGCCCTGATCAAGAAGCTGAACCACCTGGTGCTGAAGGACAAGGCCGACGA
TGAGATCGGCTCTTACAAGAATGCCCTGCAGCTGACCAACAATTTCACAGATCTG
AAGAGCATCGGCAAGCAGACCGGCTTCCTGTTTTATGTGCCCGCCTGGAACACCT
CTAAGATCGACCCTGAGACAGGCTTTGTGGATCTGCTGAAGCCAAGATACGAGA
ACATCGCCCAGAGCCAGGCCTTCTTTGGCAAGTTCGACAAGATCTGCTATAATGC
CGACAAGGATTACTTCGAGTTTCACATCGACTACGCCAAGTTTACCGATAAGGCC
AAGAATAGCCGCCAGATCTGGACAATCTGTTCCCACGGCGACAAGCGGTACGTG
TACGATAAGACAGCCAACCAGAATAAGGGCGCCGCCAAGGGCATCAACGTGAAT
GATGAGCTGAAGTCCCTGTTCGCCCGCCACCACATCAACGAGAAGCAGCCCAAC
CTGGTCATGGACATCTGCCAGAACAATGATAAGGAGTTTCACAAGTCTCTGATGT
ACCTGCTGAAAACCCTGCTGGCCCTGCGGTACAGCAACGCCTCCTCTGACGAGG
ATTTCATCCTGTCCCCCGTGGCAAACGACGAGGGCGTGTTCTTTAATAGCGCCCT
GGCCGACGATACACAGCCTCAGAATGCCGATGCCAACGGCGCCTACCACATCGC
CCTGAAGGGCCTGTGGCTGCTGAATGAGCTGAAGAACTCCGACGATCTGAACAA
GGTGAAGCTGGCCATCGACAATCAGACCTGGCTGAATTTCGCCCAGAACAGGAA
AAGGCCGGCGGCCACGAAAAAGGCCGGCCAGGCAAAAAAGAAAAAGGGATCCT
ACCCATACGATGTTCCAGATTACGCTTATCCCTACGACGTGCCTGATTATGCATA
CCCATATGATGTCCCCGACTATGCCTAA

What is claimed is:

1. A method of inhibiting a Cas12a polypeptide, the method comprising,
contacting a Cas12a-inhibiting polypeptide to the Cas12a polypeptide, wherein:
the Cas12a-inhibiting polypeptide is substantially (e.g., at least 60%, 70%, 80%, 90%, 95%, 99%) identical to any one or more of SEQ ID NO: 2-53;
thereby inhibiting the Cas12a polypeptide.

2. The method of claim 1, wherein the contacting occurs in vitro.

3. The method of claim 1, wherein the contacting occurs in a cell.

4. The method of claim 3, wherein the contacting comprises introducing the Cas12a-inhibiting polypeptide into the cell.

5. The method of claim 4, wherein the Cas12a-inhibiting polypeptide is heterologous to the cell.

6. The method of claim 4, wherein the Cas12a polypeptide is present in the cell prior to the contacting.

7. The method of claim 4, wherein the Cas12a-inhibiting polypeptide comprises one of SEQ ID NO: 2-53.

8. The method of claim 4, wherein the cell comprises the Cas12a polypeptide before the introducing.

9. The method of claim 8, wherein the cell comprises a heterologous expression cassette comprising a promoter operably linked to a polynucleotide encoding the Cas12a polypeptide.

10. The method of claim 9, wherein the promoter is inducible and the method comprises contacting the cell with an agent or condition that induces expression of the Cas12a polypeptide in the cell prior to the introducing.

11. The method of claim 4, wherein the Cas12a polypeptide is introduced to the cell when or after the Cas12a-inhibiting polypeptide is introduced to the cell.

12. The method of claim 11, wherein the promoter is inducible and the method comprises contacting the cell with an agent or condition that induces expression of the Cas12a polypeptide in the cell after the introducing.

13. The method of claim 4, wherein the introducing comprises expressing the Cas12a-inhibiting polypeptide in the cell from an expression cassette that is present in the cell and heterologous to the cell, wherein the expression cassette comprises a promoter operably linked to a polynucleotide encoding the Cas12a-inhibiting polypeptide.

14. The method of claim 13, wherein the promoter is an inducible promoter and the introducing comprises contacting the cell with an agent that induces expression of the Cas12a-inhibiting polypeptide.

15. The method of claim 4, wherein the introducing comprises introducing an RNA encoding the Cas12a-inhibiting polypeptide into the cell and expressing the Cas12a-inhibiting polypeptide in the cell from the RNA.

16. The method of claim 4, wherein the introducing comprises inserting the Cas12a-inhibiting polypeptide into the cell or contacting the cell with the Cas12a-inhibiting polypeptide.

17. The method of any of claims 4-16, wherein the cell is a eukaryotic cell.

18. The method of claim 17, wherein the cell is a mammalian cell.

19. The method of claim 18, wherein the cell is a human cell.

20. The method of any of claims 18-19, wherein the cell is a blood or an induced pluripotent stem cell.

21. The method of any of claims 18-20, wherein the method occurs ex vivo.

22. The method of claim 21, wherein the cells are introduced into a mammal after the introducing and contacting.

23. The method of claim 22, wherein the cells are autologous to the mammal.

24. The method of any of claims 4-16, wherein the cell is a prokaryotic cell.

25. A cell comprising a Cas12a-inhibiting polypeptide, wherein the Cas12a-inhibiting polypeptide is heterologous to the cell and the Cas12a-inhibiting polypeptide is substantially (e.g., at least 60%, 70%, 80%, 90%, 95%, 99%) identical to any one or more of SEQ ID NO: 2-53.

26. The cell of any of claim 25, wherein the cell is a eukaryotic cell.

27. The method of claim 26, wherein the cell is a mammalian cell.

28. The method of claim 27, wherein the cell is a human cell.

29. The method of any of claim 25, wherein the cell is a prokaryotic cell.

30. A polynucleotide comprising a nucleic acid encoding a Cas12a-inhibiting polypeptide, wherein the Cas12a-inhibiting polypeptide is substantially (e.g., at least 60%, 70%, 80%, 90%, 95%, 99%) identical to any one or more of SEQ ID NO: 2-53.

31. The polynucleotide of claim 30, comprising an expression cassette, the expression cassette comprising a promoter operably linked to the nucleic acid.

32. The polynucleotide of claim 31, wherein the promoter is heterologous to the polynucleotide encoding the Cas12a-inhibiting polypeptide.

33. The polynucleotide of claim 31 or 32, wherein the promoter is inducible.

34. The polynucleotide of claim 30, wherein the polynucleotide is DNA or RNA.

35. A vector comprising the expression cassette of any of claims 31-33.

36. The vector of claim 35, wherein the vector is a viral vector.

37. A Cas12a-inhibiting polypeptide, wherein the Cas12a-inhibiting polypeptide comprises an amino acid sequence substantially (e.g., at least 60%, 70%, 80%, 90%, 95%, 99%) identical to any one or more of SEQ ID NO: 2-53.

38. The Cas12a-inhibiting polypeptide of claim 37, wherein the amino acid sequence is linked to a heterologous protein sequence.

39. The Cas12a-inhibiting polypeptide of claim 38, wherein the heterologous protein sequence extends the circulating half-life of the polypeptide.

40. The Cas12a-inhibiting polypeptide of claim 39, wherein the amino acid sequence is linked to an antibody Fc domain or human serum albumin.

41. The Cas12a-inhibiting polypeptide of claim 37, wherein the polypeptide is PEGylated or comprises at least one non-naturally-encoded amino acid.

42. A pharmaceutical composition comprising the polynucleotide of any of claims 30-33 or the polypeptide of any of claims 37-41.

43. A delivery vehicle comprising the polynucleotide of any of claims 30-34 or the polypeptide of any of claims 37-41.

44. The delivery vehicle of claim 43, wherein the delivery vehicle is a liposome or nanoparticle.
